SEQUENCE LISTING (188 sequences)

SEQ ID NO: 1 - protein, 37 aa, synthetic construct
QMDMRCSASVECKQKCLKAIGSIFGKCMNKKCKCYPR

SEQ ID NO: 2 - protein, 37 aa, synthetic construct
QMDMRCSASVECKQKCLKAIGRGFGKCMNKKCKCYPR

SEQ ID NO: 3 - protein, 37 aa, synthetic construct
NMDMRCSASVECKQKCLKAIGSIFGKCMNKKCKCYPR

SEQ ID NO: 4 - protein, 37 aa, synthetic construct
NMDMRCSASVECKQKCLKAIGRGFGKCMNKKCKCYPR

SEQ ID NO: 5 - protein, 36 aa, synthetic construct
MDMRCSASVECKQKCLKAIGSIFGKCMNKKCKCYPR

SEQ ID NO: 6 - protein, 35 aa, synthetic construct
DMRCSASVECKQKCLKAIGSIFGKCMNKKCKCYPR

SEQ ID NO: 7 - protein, 37 aa, synthetic construct
  MOD_RES 2: note = Nle
  MOD_RES 4: note = Nle
  MOD_RES 28: note = Nle
PXEXRCYASVECKQKCLAAIGSIFGKCXNKKCKCYPR

SEQ ID NO: 8 - protein, 37 aa, synthetic construct
NIDMRCSASVECKQKCLKAIGSIFGKCMNKKCKCYPR

SEQ ID NO: 9 - protein, 37 aa, synthetic construct
NMDVRCSASVECKQKCLKAIGSIFGKCMNKKCKCYPR

SEQ ID NO: 10 - protein, 37 aa, synthetic construct
NMDMRCSASVECKQKCKDAIGSIFGKCMNKKCKCYPR

SEQ ID NO: 11 - protein, 39 aa, synthetic construct
GGNMDMRCSASVECKQKCLKAIGSIFGKCMNKKCKCYPR

SEQ ID NO: 12 - protein, 37 aa, synthetic construct
NMEMRCSASVECKQKCLKAIGSIFGKCMNKKCKCYPR

SEQ ID NO: 13 - protein, 39 aa, synthetic construct
SGNMDMRCSASVECKQKCLKAIGSIFGKCMNKKCKCYPR

SEQ ID NO: 14 - protein, 37 aa, synthetic construct
  MOD_RES 2: note = Nle
NXDMRCSASVECKQKCLKAIGSIFGKCMNKKCKCYPR

SEQ ID NO: 15 - protein, 37 aa, synthetic construct
  MOD_RES 4: note = Nle
NMDXRCSASVECKQKCLKAIGSIFGKCMNKKCKCYPR

SEQ ID NO: 16 - protein, 37 aa, synthetic construct
  MOD_RES 28: note = Nle
NMDMRCSASVECKQKCLKAIGSIFGKCXNKKCKCYPR

SEQ ID NO: 17 - protein, 37 aa, synthetic construct
NMDMRCSASVECKVKCLKAIGSIFGKCMNKKCKCYPR

SEQ ID NO: 18 - protein, 37 aa, synthetic construct
NMDMRCSASVECKQLCLKAIGSIFGKCMNKKCKCYPR

SEQ ID NO: 19 - protein, 37 aa, synthetic construct
NMDMRCSASVECKQKCKKAIGSIFGKCMNKKCKCYPR

SEQ ID NO: 20 - protein, 37 aa, synthetic construct
NMDMRCSASVECKQKCLDAIGSIFGKCMNKKCKCYPR

SEQ ID NO: 21 - protein, 37 aa, synthetic construct
NMDMRCSASVECKQKCLKAIRSIFGKCMNKKCKCYPR

SEQ ID NO: 22 - protein, 37 aa, synthetic construct
NMDMRCSASVECKQKCLKAIESIFGKCMNKKCKCYPR

SEQ ID NO: 23 - protein, 41 aa, synthetic construct
NMDMRCSASVECKQKCLKAIGSIFGKCMNKKCKCYPRRRTA

SEQ ID NO: 24 - protein, 41 aa, synthetic construct
NMDMRCSASVECKQKCLKAIGSIFGKCMNKKCKCYPRHRRK

SEQ ID NO: 25 - protein, 37 aa, synthetic construct
NMDMRCKASVECKQKCLKAIGSIFGKCMNKKCKCYPR

SEQ ID NO: 26 - protein, 37 aa, synthetic construct
NMDMRCSISVECKQKCLKAIGSIFGKCMNKKCKCYPR

SEQ ID NO: 27 - protein, 37 aa, synthetic construct
NMDMRCSASRECKQKCLKAIGSIFGKCMNKKCKCYPR

SEQ ID NO: 28 - protein, 37 aa, synthetic construct
NMDMRCSASVQCKQKCLKAIGSIFGKCMNKKCKCYPR

SEQ ID NO: 29 - protein, 37 aa, synthetic construct
NMDMRCSASVECLQKCLKAIGSIFGKCMNKKCKCYPR

SEQ ID NO: 30 - protein, 37 aa, synthetic construct
NMDMRCSASVECAQKCLKAIGSIFGKCMNKKCKCYPR

SEQ ID NO: 31 - protein, 37 aa, synthetic construct
NMDMRCSASVECKEKCLKAIGSIFGKCMNKKCKCYPR

SEQ ID NO: 32 - protein, 37 aa, synthetic construct
NMDMRCSASVECKLKCLKAIGSIFGKCMNKKCKCYPR

SEQ ID NO: 33 - protein, 37 aa, synthetic construct
NMDMRCSASVECKQKCLKAIHSIFGKCMNKKCKCYPR

SEQ ID NO: 34 - protein, 37 aa, synthetic construct
NMDMRCSASVECKQKCLKAIGSKFGKCMNKKCKCYPR

SEQ ID NO: 35 - protein, 37 aa, synthetic construct
NMDMRCSASVECKQKCLKAIGSRFGKCMNKKCKCYPR

SEQ ID NO: 36 - protein, 37 aa, synthetic construct
NMDMRCSASVECKQKCLKAIGSIFGKCMNGKCKCYPR

SEQ ID NO: 37 - protein, 37 aa, synthetic construct
NMDMRCSASVECKQKCLKAIGSIFGKCMNKKCHCYPR

SEQ ID NO: 38 - protein, 37 aa, synthetic construct
NMDMRCSASVECKQKCLKAIGSIFGKCMNKKCVCYPR

SEQ ID NO: 39 - protein, 37 aa, synthetic construct
  MOD_RES 2: note = Nle
  MOD_RES 4: note = Nle
NXDXRCSASVECKQKCLKAIGSIFGKCMNKKCKCYPR

SEQ ID NO: 40 - protein, 37 aa, synthetic construct
  MOD_RES 2: note = Nle
  MOD_RES 4: note = Nle
  MOD_RES 28: note = Nle
NXDXRCSASVECKQKCLKAIGSIFGKCXNKKCKCYPR

SEQ ID NO: 41 - protein, 37 aa, synthetic construct
  MOD_RES 2: note = Nle
  MOD_RES 4: note = Nle
  MOD_RES 28: note = Nle
PXDXRCSASVECKQKCLKAIGSIFGKCXNKKCKCYPR

SEQ ID NO: 42 - protein, 37 aa, synthetic construct
  MOD_RES 2: note = Nle
  MOD_RES 4: note = Nle
  MOD_RES 28: note = Nle
NXDXRCRASVECKQKCLKAIGSIFGKCXNKKCKCYPR

SEQ ID NO: 43 - protein, 37 aa, synthetic construct
  MOD_RES 2: note = Nle
  MOD_RES 4: note = Nle
  MOD_RES 28: note = Nle
NXDXRCSASVECEQKCLKAIGSIFGKCXNKKCKCYPR

SEQ ID NO: 44 - protein, 37 aa, synthetic construct
  MOD_RES 2: note = Nle
  MOD_RES 4: note = Nle
  MOD_RES 28: note = Nle
NXDXRCSASVECQQKCLKAIGSIFGKCXNKKCKCYPR

SEQ ID NO: 45 - protein, 37 aa, synthetic construct
  MOD_RES 2: note = Nle
  MOD_RES 4: note = Nle
  MOD_RES 28: note = Nle
NXDXRCSASVECKKKCLKAIGSIFGKCXNKKCKCYPR

SEQ ID NO: 46 - protein, 37 aa, synthetic construct
  MOD_RES 2: note = Nle
  MOD_RES 4: note = Nle
  MOD_RES 28: note = Nle
NXSXRCSASVECKQKCLKAIGSIFGKCXNKKCKCYPR

SEQ ID NO: 47 - protein, 37 aa, synthetic construct
  MOD_RES 2: note = Nle
  MOD_RES 4: note = Nle
  MOD_RES 28: note = Nle
NXDXRCSHSVECKQKCLKAIGSIFGKCXNKKCKCYPR

SEQ ID NO: 48 - protein, 37 aa, synthetic construct
  MOD_RES 2: note = Nle
  MOD_RES 4: note = Nle
  MOD_RES 28: note = Nle
NXDXRCSASVECKQSCLKAIGSIFGKCXNKKCKCYPR

SEQ ID NO: 49 - protein, 36 aa, synthetic construct
  MOD_RES 2: note = Nle
  MOD_RES 4: note = Nle
  MOD_RES 27: note = Nle
NXDXRCSASVECKQKCKAIGSIFGKCXNKKCKCYPR

SEQ ID NO: 50 - protein, 37 aa, synthetic construct
NMDMRCSASVECKQKCYKAIGSIFGKCMNKKCKCYPR

SEQ ID NO: 51 - protein, 37 aa, synthetic construct
NMDMRCSASVECKQKCRKAIGSIFGKCMNKKCKCYPR

SEQ ID NO: 52 - protein, 37 aa, synthetic construct
NMDMRCSASVECKQKCLAAIGSIFGKCMNKKCKCYPR

SEQ ID NO: 53 - protein, 37 aa, synthetic construct
NMDMRCSASVECKQKCLYAIGSIFGKCMNKKCKCYPR

SEQ ID NO: 54 - protein, 36 aa, synthetic construct
NMDMRCSASVECKQKCLAIGSIFGKCMNKKCKCYPR

SEQ ID NO: 55 - protein, 37 aa, synthetic construct
NMDMRCSASVECKQKCLKYIGSIFGKCMNKKCKCYPR

SEQ ID NO: 56 - protein, 37 aa, synthetic construct
NMDMRCSASVECKQKCLKRIGSIFGKCMNKKCKCYPR

SEQ ID NO: 57 - protein, 36 aa, synthetic construct
NMDMRCSASVECKQKCLKIGSIFGKCMNKKCKCYPR

SEQ ID NO: 58 - protein, 37 aa, synthetic construct
NMDMRCSASVECKQKCLKAYGSIFGKCMNKKCKCYPR

SEQ ID NO: 59 - protein, 37 aa, synthetic construct
NMDMRCSASVECKQKCLKAEGSIFGKCMNKKCKCYPR

SEQ ID NO: 60 - protein, 37 aa, synthetic construct
NMDMRCSASVECKQKCLKARGSIFGKCMNKKCKCYPR

SEQ ID NO: 61 - protein, 36 aa, synthetic construct
NMDMRCSASVECKQKCLKAGSIFGKCMNKKCKCYPR

SEQ ID NO: 62 - protein, 37 aa, synthetic construct
  MOD_RES 2: note = Nle
  MOD_RES 4: note = Nle
  MOD_RES 28: note = Nle
NXDXRCSASVECKQKCLKAIGSPFGKCXNKKCKCYPR

SEQ ID NO: 63 - protein, 37 aa, synthetic construct
  MOD_RES 2: note = Nle
  MOD_RES 4: note = Nle
  MOD_RES 28: note = Nle
NXDXRCSASKECKQKCLKAIGSIFGKCXNKKCKCYPR

SEQ ID NO: 64 - protein, 37 aa, synthetic construct
  MOD_RES 2: note = Nle
  MOD_RES 4: note = Nle
  MOD_RES 28: note = Nle
HXDXRCSASVECKQKCLKAIGSIFGKCXNKKCKCYPR

SEQ ID NO: 65 - protein, 37 aa, synthetic construct
  MOD_RES 2: note = Nle
  MOD_RES 4: note = Nle
  MOD_RES 28: note = Nle
YXDXRCSASVECKQKCLKAIGSIFGKCXNKKCKCYPR

SEQ ID NO: 66 - protein, 37 aa, synthetic construct
  MOD_RES 2: note = Nle
  MOD_RES 4: note = Nle
  MOD_RES 28: note = Nle
SXDXRCSASVECKQKCLKAIGSIFGKCXNKKCKCYPR

SEQ ID NO: 67 - protein, 37 aa, synthetic construct
  MOD_RES 2: note = Nle
  MOD_RES 4: note = Nle
  MOD_RES 28: note = Nle
VXDXRCSASVECKQKCLKAIGSIFGKCXNKKCKCYPR

SEQ ID NO: 68 - protein, 37 aa, synthetic construct
  MOD_RES 2: note = Nle
  MOD_RES 4: note = Nle
  MOD_RES 9: note = Abu
SXDXRCSAXVECKQKCLKAIGSIFGKCMNKKCKCYPR

SEQ ID NO: 69 - protein, 37 aa, synthetic construct
  MOD_RES 2: note = Nle
  MOD_RES 4: note = Nle
  MOD_RES 26: note = homo-Lys
SXDXRCSASVECKQKCLKAIGSIFGKCMNKKCKCYPR

SEQ ID NO: 70 - protein, 37 aa, synthetic construct
  MOD_RES 2: note = Nle
  MOD_RES 4: note = Nle
  MOD_RES 35: note = F(4-NH2)
SXDXRCSASVECKQKCLKAIGSIFGKCMNKKCKCXPR

SEQ ID NO: 71 - protein, 37 aa, synthetic construct
  MOD_RES 2: note = Nle
  MOD_RES 4: note = Nle
SXDXRCSASVECGQKCLKAIGSIFGKCMNKKCKCYPR

SEQ ID NO: 72 - protein, 37 aa, synthetic construct
  MOD_RES 2: note = Nle
  MOD_RES 4: note = Nle
SXDXRCSASVECVQKCLKAIGSIFGKCMNKKCKCYPR

SEQ ID NO: 73 - protein, 37 aa, synthetic construct
  MOD_RES 2: note = Nle
  MOD_RES 4: note = Nle
  MOD_RES 14: note = 2-Amino-5-carboxypentanoyl
SXDXRCSASVECKXKCLKAIGSIFGKCMNKKCKCYPR

SEQ ID NO: 74 - protein, 36 aa, synthetic construct
  MOD_RES 2: note = Nle
  MOD_RES 4: note = Nle
SXDXRCSASVECKQKCLKAIGSIFGKCMNKKCKCYQ

SEQ ID NO: 75 - protein, 37 aa, synthetic construct
  MOD_RES 2: note = Nle
  MOD_RES 4: note = Nle
SXDXRCSASVECKQKCLKAIGSIFGKCMNKKCRCYPR

SEQ ID NO: 76 - protein, 37 aa, synthetic construct
  MOD_RES 2: note = Nle
SXDERCSASVECKQKCLKAIGSIFGKCMNKKCKCYPR

SEQ ID NO: 77 - protein, 37 aa, synthetic construct
  MOD_RES 2: note = Nle
  MOD_RES 4: note = Nle
SXDXRCSASVECKQKCLGAIGSIFGKCMNKKCKCYPR

SEQ ID NO: 78 - protein, 37 aa, synthetic construct
  MOD_RES 2: note = Nle
  MOD_RES 4: note = Nle
SXDXRCSASVECKQKCLVAIGSIFGKCMNKKCKCYPR

SEQ ID NO: 79 - protein, 37 aa, synthetic construct
  MOD_RES 2: note = Nle
  MOD_RES 4: note = Nle
SXDXRCSASVECAQSCLKAIGSIFGKCMNKKCKCYPR

SEQ ID NO: 80 - protein, 37 aa, synthetic construct
  MOD_RES 2: note = Nle
  MOD_RES 4: note = Nle
SXDXRCSASVECAQKCLAAIGSIFGKCMNKKCKCYPR

SEQ ID NO: 81 - protein, 37 aa, synthetic construct
  MOD_RES 2: note = Nle
  MOD_RES 4: note = Nle
SXDXRCSASVECAQLCLAAIGSIFGKCMNKKCKCYPR

SEQ ID NO: 82 - protein, 37 aa, synthetic construct
  MOD_RES 2: note = Nle
  MOD_RES 4: note = Nle
  MOD_RES 7: note = 2,4-Diaminobutanoyl
  MOD_RES 28: note = Nle
PXEXRCXASVECKQKCLAAIGSIFGKCXNKKCKCYPR

SEQ ID NO: 83 - protein, 37 aa, synthetic construct
  MOD_RES 2: note = Nle
  MOD_RES 4: note = Nle
  MOD_RES 28: note = Nle
PXDXRCSASVECKQKCLKAIGCIFGKCXNKKCKCYPC

SEQ ID NO: 84 - protein, 37 aa, synthetic construct
  MOD_RES 2: note = Nle
  MOD_RES 4: note = Nle
  MOD_RES 28: note = Nle
SXDXRCSASVECKEKCLQAIGSIFGKCXNKKCKCYPR

SEQ ID NO: 85 - protein, 37 aa, synthetic construct
  MOD_RES 2: note = Nle
  MOD_RES 4: note = Nle
  MOD_RES 18: note = Homo-Gln
  MOD_RES 28: note = Nle
PXDXRCSASVECKEKCLXAIGSIFGKCXNKKCKCYPR

SEQ ID NO: 86 - protein, 32 aa, synthetic construct
  MOD_RES 23: note = Nle
CSASVECKQKCLKAIGCIFGKCXNKKCKCYPC

SEQ ID NO: 87 - protein, 37 aa, synthetic construct
  MOD_RES 2: note = Nle
  MOD_RES 4: note = Nle
  MOD_RES 28: note = Nle
SXDXRCSASVECKQKCLAAIGCIFGKCXNKKCKCYPC

SEQ ID NO: 88 - protein, 37 aa, synthetic construct; identical to SEQ ID NO: 82
  MOD_RES 2: note = Nle
  MOD_RES 4: note = Nle
  MOD_RES 7: note = 2-Amino-3-guanidinopropionyl
  MOD_RES 28: note = Nle

SEQ ID NO: 89 - protein, 37 aa, synthetic construct
  MOD_RES 2: note = Nle
  MOD_RES 4: note = Nle
SXDXRCSALVECKQKCLKAIGSIFGKCMNKKCKCYPR

SEQ ID NO: 90 - protein, 37 aa, synthetic construct
  MOD_RES 2: note = Nle
  MOD_RES 4: note = Nle
SXDXRCSAVVECKQKCLKAIGSIFGKCMNKKCKCYPR

SEQ ID NO: 91 - protein, 37 aa, synthetic construct; identical to SEQ ID NO: 70
  MOD_RES 2: note = Nle
  MOD_RES 4: note = Nle
  MOD_RES 35: note = F(4-F)

SEQ ID NO: 92 - protein, 37 aa, synthetic construct; identical to SEQ ID NO: 70
  MOD_RES 2: note = Nle
  MOD_RES 4: note = Nle
  MOD_RES 35: note = F(4-NO2)

SEQ ID NO: 93 - protein, 37 aa, synthetic construct; identical to SEQ ID NO: 70
  MOD_RES 2: note = Nle
  MOD_RES 4: note = Nle
  MOD_RES 35: note = F(4-CH3)

SEQ ID NO: 94 - protein, 36 aa, synthetic construct
  MOD_RES 1: note = Nle
  MOD_RES 3: note = Nle
  MOD_RES 27: note = Nle
XDXRCSASVECKQKCLKAIGSIFGKCXNKKCKCYPR

SEQ ID NO: 95 - protein, 37 aa, synthetic construct
  MOD_RES 4: note = Nle
  MOD_RES 28: note = Nle
NIDXRCSASVECKQKCLKAIGSIFGKCXNKKCKCYPR

SEQ ID NO: 96 - protein, 37 aa, synthetic construct
  MOD_RES 4: note = Nle
  MOD_RES 28: note = Nle
PIEXRCSASVECKQKCLKAIGSIFGKCXNKKCKCYPR

SEQ ID NO: 97 - protein, 37 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPR

SEQ ID NO: 98 - protein, 37 aa, synthetic construct
  MOD_RES 2: note = Nle
  MOD_RES 4: note = Nle
  MOD_RES 28: note = Nle
  MOD_RES 9: note = Orn
PXEXRCSAXVECKQKCLAAIGSIFGKCXNKKCKCYPR

SEQ ID NO: 99 - protein, 37 aa, synthetic construct
  MOD_RES 2: note = Nle
  MOD_RES 4: note = Nle
  MOD_RES 28: note = Nle
PXEXRCSASVECKQKCLLAIGSIFGKCXNKKCKCYPR

SEQ ID NO: 100 - protein, 37 aa, synthetic construct
  MOD_RES 28: note = Nle
PIDERCSASVECKQKCLAAIGSIFGKCXNKKCKCYPR

SEQ ID NO: 101 - protein, 37 aa, synthetic construct
  MOD_RES 4: note = Nle
  MOD_RES 28: note = Nle
PIEXRCSASVECKQKCLAAIGSIFGKCXNKKCKCYPR

SEQ ID NO: 102 - protein, 37 aa, synthetic construct
  MOD_RES 2: note = Nle
  MOD_RES 4: note = Nle
PXDXRCSASVECAQKCLAAIGSIFGKCMNKKCKCYPR

SEQ ID NO: 103 - protein, 37 aa, synthetic construct
  MOD_RES 2: note = Nle
  MOD_RES 4: note = Nle
  MOD_RES 28: note = Nle
PXEXRCSASVECAQKCLAAIGSIFGKCXNKKCKCYPR

SEQ ID NO: 104 - protein, 32 aa, synthetic construct
  MOD_RES 23: note = Nle
CSASVECKQKCLKAIGSIFGKCXNKKCKCYPR

SEQ ID NO: 105 - protein, 37 aa, synthetic construct; identical to SEQ ID NO: 82
  MOD_RES 2: note = Nle
  MOD_RES 4: note = Nle
  MOD_RES 28: note = Nle
  MOD_RES 7: note = 2,3-diaminopropanoyl

SEQ ID NO: 106 - protein, 33 aa, synthetic construct
  MOD_RES 24: note = Nle
RCSASVECKQKCLKAIGSIFGKCXNKKCKCYPR

SEQ ID NO: 107 - protein, 34 aa, synthetic construct
  MOD_RES 25: note = Nle
SKCSASVECKQKCLKAIGSIFGKCXNKKCKCYPR

SEQ ID NO: 108 - protein, 34 aa, synthetic construct
  MOD_RES 25: note = Nle
LRCSASVECKQKCLKAIGSIFGKCXNKKCKCYPR

SEQ ID NO: 109 - protein, 32 aa, synthetic construct
  MOD_RES 23: note = Nle
CSASVECKQKCLKAIGSIFGKCXNKKCKCYPS

SEQ ID NO: 110 - protein, 31 aa, synthetic construct
  MOD_RES 23: note = Nle
CSASVECKQKCLKAIGSIFGKCXNKKCKCYS

SEQ ID NO: 111 - protein, 31 aa, synthetic construct
  MOD_RES 23: note = Nle
CSASVECKQKCLKAIGSIFGKCXNKKCKCYG

SEQ ID NO: 112 - protein, 30 aa, synthetic construct
  MOD_RES 23: note = Nle
CSASVECKQKCLKAIGSIFGKCXNKKCKCY

SEQ ID NO: 113 - protein, 37 aa, synthetic construct; identical to SEQ ID NO: 82
  MOD_RES 2: note = Nle
  MOD_RES 4: note = Nle
  MOD_RES 28: note = Nle
  MOD_RES 7: note = Orn

SEQ ID NO: 114 - protein, 37 aa, synthetic construct
  MOD_RES 2: note = Nle
  MOD_RES 4: note = Nle
  MOD_RES 28: note = Nle
PXEXRCHASVECKQKCLAAIGSIFGKCXNKKCKCYPR

SEQ ID NO: 115 - protein, 34 aa, synthetic construct
  MOD_RES 25: note = Nle
LRCSASVECKQKCLAAIGSIFGKCXNKKCKCYPR

SEQ ID NO: 116 - protein, 34 aa, synthetic construct
LRCSASVECKQKCLAAIGSIFGKCMNKKCKCYPR

SEQ ID NO: 117 - protein, 32 aa, synthetic construct
CSASVECKQKCLKAIGSIFGKCMNKKCKCYPR

SEQ ID NO: 118 - protein, 37 aa, synthetic construct
  MOD_RES 2: note = Nle
  MOD_RES 4: note = Nle
  MOD_RES 28: note = Nle
PXEXRCSASVECKEKCLAAIGSIFGKCXNKKCKCYPR

SEQ ID NO: 119 - protein, 37 aa, synthetic construct; identical to SEQ ID NO: 97
  MOD_RES 2: note = Nle
  MOD_RES 4: note = Nle
  MOD_RES 28: note = Nle
  SITE 1: note = D-proline

SEQ ID NO: 120 - protein, 36 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYR

SEQ ID NO: 121 - protein, 36 aa, synthetic construct
  MOD_RES 1: note = Nle
  MOD_RES 3: note = Nle
  MOD_RES 27: note = Nle
XEXRCSASVECKQKCLAAIGSIFGKCXNKKCKCYPR

SEQ ID NO: 122 - protein, 38 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRR

SEQ ID NO: 123 - protein, 38 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRY

SEQ ID NO: 124 - protein, 38 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRL

SEQ ID NO: 125 - protein, 38 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRH

SEQ ID NO: 126 - protein, 38 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRE

SEQ ID NO: 127 - protein, 39 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRKS

SEQ ID NO: 128 - protein, 39 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRFE

SEQ ID NO: 129 - protein, 39 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRHR

SEQ ID NO: 130 - protein, 39 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRAK

SEQ ID NO: 131 - protein, 37 aa, synthetic construct
  MOD_RES 2: note = Nle
  MOD_RES 4: note = Nle
  MOD_RES 28: note = Nle
  MOD_RES 37: note = (4-amino-5-hydroxypentyl)guanidine
PXEXRCSASVECKQKCLAAIGSIFGKCXNKKCKCYPX

SEQ ID NO: 132 - protein, 38 aa, synthetic construct
  MOD_RES 2: note = Nle
  MOD_RES 4: note = Nle
  MOD_RES 28: note = Nle
  MOD_RES 38: note = 4-amino-5-hydroxypentanamide
PXEXRCSASVECKQKCLAAIGSIFGKCXNKKCKCYPRX

SEQ ID NO: 133 - protein, 39 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRST

SEQ ID NO: 134 - protein, 39 aa, synthetic construct
  MOD_RES 2: note = Nle
  MOD_RES 4: note = Nle
  MOD_RES 28: note = Nle
PXEXRCSASVECKQKCLAAIGSIFGKCXNKKCKCYPRRY

SEQ ID NO: 135 - protein, 37 aa, synthetic construct
  MOD_RES 2: note = Nle
  MOD_RES 4: note = Nle
  MOD_RES 28: note = Nle
PXEXRCSSSVECKQKCLAAIGSIFGKCXNKKCKCYPR

SEQ ID NO: 136 - protein, 37 aa, synthetic construct
  MOD_RES 2: note = Nle
  MOD_RES 4: note = Nle
  MOD_RES 28: note = Nle
PXEXRCSLSVECKQKCLAAIGSIFGKCXNKKCKCYPR

SEQ ID NO: 137 - protein, 37 aa, synthetic construct
  MOD_RES 2: note = Nle
  MOD_RES 4: note = Nle
  MOD_RES 28: note = Nle
PXEXRCSAPVECKQKCLAAIGSIFGKCXNKKCKCYPR

SEQ ID NO: 138 - protein, 37 aa, synthetic construct
  MOD_RES 2: note = Nle
  MOD_RES 4: note = Nle
  MOD_RES 28: note = Nle
PXEXRCSASPECKQKCLAAIGSIFGKCXNKKCKCYPR

SEQ ID NO: 139 - protein, 37 aa, synthetic construct
  MOD_RES 2: note = Nle
  MOD_RES 4: note = Nle
  MOD_RES 28: note = Nle
PXEXRCSASQECKQKCLAAIGSIFGKCXNKKCKCYPR

SEQ ID NO: 140 - protein, 37 aa, synthetic construct
  MOD_RES 2: note = Nle
  MOD_RES 4: note = Nle
  MOD_RES 28: note = Nle
PXEXRCSASVECLQKCLAAIGSIFGKCXNKKCKCYPR

SEQ ID NO: 141 - protein, 37 aa, synthetic construct
  MOD_RES 2: note = Nle
  MOD_RES 4: note = Nle
  MOD_RES 28: note = Nle
PXEXRCSASVECKQPCLAAIGSIFGKCXNKKCKCYPR

SEQ ID NO: 142 - protein, 37 aa, synthetic construct
  MOD_RES 2: note = Nle
  MOD_RES 4: note = Nle
  MOD_RES 28: note = Nle
PXEXRCEASVECKQKCLAAIGSIFGKCXNKKCKCYPR

SEQ ID NO: 143 - protein, 37 aa, synthetic construct
  MOD_RES 2: note = Nle
  MOD_RES 4: note = Nle
  MOD_RES 28: note = Nle
PXEXRCFASVECKQKCLAAIGSIFGKCXNKKCKCYPR

SEQ ID NO: 144 - protein, 37 aa, synthetic construct
  MOD_RES 2: note = Nle
  MOD_RES 4: note = Nle
  MOD_RES 28: note = Nle
PXEXRCSYSVECKQKCLAAIGSIFGKCXNKKCKCYPR

SEQ ID NO: 145 - protein, 37 aa, synthetic construct
  MOD_RES 2: note = Nle
  MOD_RES 4: note = Nle
  MOD_RES 28: note = Nle
PXEXRCSAFVECKQKCLAAIGSIFGKCXNKKCKCYPR

SEQ ID NO: 146 - protein, 37 aa, synthetic construct; identical to SEQ ID NO: 98
  MOD_RES 2: note = Nle
  MOD_RES 4: note = Nle
  MOD_RES 9: note = 2,3-Diaminopropanoyl
  MOD_RES 28: note = Nle

SEQ ID NO: 147 - protein, 37 aa, synthetic construct
  MOD_RES 2: note = Nle
  MOD_RES 4: note = Nle
  MOD_RES 28: note = Nle
  MOD_RES 7: note = 2,4-Diaminobutanoyl
PXEXRCXASVECAQSCLAAIGSIFGKCXNGKCKCYPR

SEQ ID NO: 148 - protein, 37 aa, synthetic construct
  MOD_RES 2: note = Nle
  MOD_RES 4: note = Nle
  MOD_RES 28: note = Nle
PXEXRCRASVECAQSCLAAIGSIFGKCXNGKCKCYPR

SEQ ID NO: 149 - protein, 37 aa, synthetic construct
  MOD_RES 2: note = Nle
  MOD_RES 4: note = Nle
  MOD_RES 28: note = Nle
PXEXRCSASRECAQSCLAAIGSIFGKCXNGKCKCYPR

SEQ ID NO: 150 - protein, 37 aa, synthetic construct
  MOD_RES 2: note = Nle
  MOD_RES 4: note = Nle
  MOD_RES 28: note = Nle
PXEXRCSASVECKQKCLAKIGSIFGKCXNKKCKCYPR

SEQ ID NO: 151 - protein, 35 aa, synthetic construct
RSCIDTIPKSRCTAFQCKHSMKYRLSFCRKTCGTC

SEQ ID NO: 152 - protein, 37 aa, synthetic construct
  MOD_RES 1: note = phosphotyrosyl
  MOD_RES 2: note = 8-Amino-3,6-dioxaoctanoyl
XXRSCIDTIPKSRCTAFQCKHSMKYRLSFCRKTCGTC

SEQ ID NO: 153 - protein, 34 aa, synthetic construct
INVKCSLPQQCIKPCKDAGMRFGKCMNKKCRCYS

SEQ ID NO: 154 - protein, 36 aa, synthetic construct
AAAISCVGSPECPPKCRAQGCKNGKCMNRKCKCYYC

SEQ ID NO: 155 - protein, 5 aa, synthetic construct
QMDMR

SEQ ID NO: 156 - protein, 5 aa, synthetic construct
NMDMR

SEQ ID NO: 157 - protein, 4 aa, synthetic construct
RRTK

SEQ ID NO: 158 - protein, 4 aa, synthetic construct
RRTA

SEQ ID NO: 159 - protein, 4 aa, synthetic construct
HRRK

SEQ ID NO: 160 - protein, 4 aa, synthetic construct
QSKA

SEQ ID NO: 161 - protein, 4 aa, synthetic construct
AGPR

SEQ ID NO: 162 - protein, 4 aa, synthetic construct
RSRT

SEQ ID NO: 163 - protein, 4 aa, synthetic construct
RHKR

SEQ ID NO: 164 - protein, 4 aa, synthetic construct
GGKR

SEQ ID NO: 165 - protein, 4 aa, synthetic construct
PKTA

SEQ ID NO: 166 - protein, 4 aa, synthetic construct
TDAR

SEQ ID NO: 167 - protein, 4 aa, synthetic construct
HRQQ

SEQ ID NO: 168 - protein, 4 aa, synthetic construct
RPRH

SEQ ID NO: 169 - protein, 4 aa, synthetic construct
ARNA

SEQ ID NO: 170 - protein, 4 aa, synthetic construct
TGRK

SEQ ID NO: 171 - protein, 4 aa, synthetic construct
HERT

SEQ ID NO: 172 - protein, 4 aa, synthetic construct
NTRT

SEQ ID NO: 173 - protein, 4 aa, synthetic construct
QRNG

SEQ ID NO: 174 - protein, 4 aa, synthetic construct
AHRN

SEQ ID NO: 175 - protein, 4 aa, synthetic construct
PRSA

SEQ ID NO: 176 - protein, 4 aa, synthetic construct
QRQS

SEQ ID NO: 177 - protein, 4 aa, synthetic construct
QRRK

SEQ ID NO: 178 - protein, 4 aa, synthetic construct
ARAK

SEQ ID NO: 179 - protein, 4 aa, synthetic construct
AKRD

SEQ ID NO: 180 - protein, 4 aa, synthetic construct
RDKT

SEQ ID NO: 181 - skipped

SEQ ID NO: 182 - protein, 4 aa, synthetic construct
RAKR

SEQ ID NO: 183 - protein, 4 aa, synthetic construct
QRTR

SEQ ID NO: 184 - protein, 4 aa, synthetic construct
ATRH

SEQ ID NO: 185 - protein, 4 aa, synthetic construct
ARRS

SEQ ID NO: 186 - protein, 4 aa, synthetic construct
AKTR

SEQ ID NO: 187 - protein, 4 aa, synthetic construct
NRQR

SEQ ID NO: 188 - protein, 4 aa, synthetic construct
PRNT

(19) United States
(12) Patent Application Publication
(10) Pub. No.: US 2026/0325605 A1
(43) Pub. Date: Oct. 22, 2026

(54) INHIBITORS

(71) Applicant: Zealand Pharma A/S, Søborg (DK)

(72) Inventors: Carola Wenander, Søborg (DK); Anneline Nansen, Søborg (DK)

(21) Appl. No.: 19/116,622

(22) PCT Filed: Oct. 18, 2023
(86) PCT No.: PCT/EP2023/079008

§ 371 Date: Mar. 28, 2025

(30) Foreign Application Priority Data
Oct. 18, 2022 (EP) 22202304.6
Jul. 10, 2023 (EP) 23184483.8

(51) Int. Cl. A61K 38/17; A61P 19/02; A61P 37/06

(52) U.S. Cl. CPC A61K 38/1767; A61P 19/02; A61P 37/06

(57) ABSTRACT

The present invention relates to use of inhibitors of the potassium channel Kv1.3 in therapeutic methods, in particular wherein the methods comprise administering the inhibitor to a subject at particular intervals.

### FIELD OF THE INVENTION

The present invention relates to use of inhibitors of the potassium channel Kv1.3 in therapeutic methods, in particular wherein the methods comprise administering the inhibitor to a subject at particular intervals.

### BACKGROUND TO THE INVENTION

Ion channels are membrane proteins which form pores in biological membranes, permitting (and regulating) the flow of ions across the relevant membrane. There are numerous different types of ion channel, which may be classified in various ways, such as by the species of ions to which they provide passage, the way in which passage of ions is regulated or “gated” (e.g. “ligand-gated” or “voltage-gated”), and their cellular or sub-cellular localisation.

Potassium channels fall into four major classes, namely voltage-gated potassium channels, calcium-activated potassium channels, inwardly rectifying potassium channels, and tandem pore domain potassium channels.

The voltage-gated potassium channels, like other voltage gated channels, open or close in response to transmembrane voltages. They represent a complex family with diverse biological functions, including the regulation of neurotransmitter release, heart rate, insulin secretion, neuronal excitability, epithelial electrolyte transport, smooth muscle contraction, and cell volume.

The Kv1.3 (potassium voltage-gated channel subfamily A member 3) channel is expressed on T cells and plays a role in regulating T cell activation. Inhibitors of Kv1.3 have been shown to inhibit proliferation of activated T cells in vitro (reviewed in Cahalan and Chandy, Immunol. Rev. 231:59-87, 2009), and to inhibit T cell-dependent disease progression in various experimental models of autoimmune disease including experimental autoimmune encephalomyelitis (EAE), experimental arthritis, delayed-type hypersensitivity (DTH), allergic contact dermatitis and glomerulonephritis. See, for example, Rangaraju et al. (Expert Opin. Ther. Targets 13:909-24, 2009); Beeton et al. (Proc. Natl. Acad. Sci. USA. 103:17414-9, 2006); Koo et al. (J. Immunol. 158:5120-8, 1997); Hyodo et al. (Am. J. Physiol. Renal Physiol. 299: F1258-69, 2010). WO 2016/112208 describes topical application of Kv1.3 blockers for the treatment of skin and mucosal inflammation.

Inhibitors of Kv1.3 have been shown to inhibit proliferation of activated T cells and to have a beneficial effect in various experimental models of disease. Without wishing to be bound by theory, it is believed that cellular efflux of potassium via the Kv1.3 channel is required to sustain calcium influx required for T-cell activation.

Kv1.3 is overexpressed in Gad5/insulin-specific T cells from patients with new onset type 1 diabetes, in myelin-specific T cells from MS patients and in T cells from the synovium of rheumatoid arthritis patients (Beeton et al., Proc Natl Acad Sci USA 103:17414-9, 2006), in breast cancer specimens (Abdul et al., Anticancer Res 23:3347, 2003) and prostate cancer cell lines (Fraser et al., Pflugers Arch 446:559, 2003).

Positive outcomes in animal models with Kv1.3 inhibitors have been described in hypersensitivity models to ovalbumin and tetanus toxoid (Beeton et al., Mol Pharmacol 67:1369, 2005; Koo et al., Clin Immunol 197:99, 1999), models for multiple sclerosis such as rat adoptive-transfer experimental autoimmune encephalomyelitis (AT-EAE) model (Beeton et al., Proc Natl Acad Sci USA 103:17414-9, 2006), inflammatory bone resorption model (Valverde et al., J Bone Mineral Res 19:155, 2004), models for arthritis (Beeton et al., Proc Natl Acad Sci 103: 17414, 2006; Tarcha et al., J. Pharmacol. Exp. Ther. 342: 642, 2012) and obesity, diabetes and metabolic disorders (Xu et al., Hum Mol Genet 12:551, 2003; Xu et al., Proc Natl Acad Sci 101: 3112, 2004). Topical application of Kv1.3 blockers has been proposed for the treatment of skin and mucosal inflammation.

Thus, Kv1.3 inhibitors have considerable potential for use in treatment of diseases and disorders, particularly inflammatory disorders such as autoimmune disorders.

WO 2015/013330 proposes use of Kv1.3 blocker peptides for treatment of ophthalmic conditions, such as dry eye and uveitis, including when caused by autoimmune conditions such as Sjogren's syndrome.

Kv1.3 inhibitors may also have beneficial metabolic effects, e.g. in relation to energy homeostasis, body weight regulation, and glucose control. Kv1.3 knock-out (Kv1.3(−/−)) mice exhibit reduced weight gain, higher insulin sensitivity, and reduced plasma glucose levels in response to a high fat diet as compared to control littermates (Xu et al., Hum. Mol. Genet. 12:551-9, 2003). Further, Kv1.3 inhibitors have been shown to increase expression in skeletal muscle and adipose tissue of glucose transporter 4 (GLUT4), to increase insulin sensitivity in normal and ob/ob obese mice, and to increase glucose uptake in primary adipocytes in vitro (Xu et al., Proc. Natl. Acad. Sci. USA 101:3112-7, 2004). In humans, a single nucleotide polymorphism (SNP) in the Kv1.3 gene has also been associated with decreased insulin sensitivity and impaired glucose tolerance (Tschritter, Clin Endocrinol Metab 91: 654-8, 2006).

Kv1.3 is also expressed in proliferating human and mouse smooth muscle cells. Inhibitors of Kv1.3 may be effective in smooth muscle proliferative disorders such as restenosis, e.g. in patients following vascular surgery (e.g. angioplasty). Kv1.3 inhibitors have been shown to inhibit calcium entry, reduce smooth muscle cell migration, and inhibit neointimal hyperplasia in ex vivo human vein samples (Cheong et al., Cardiovasc. Res. 89:282-9, 2011).

Further evidence suggests that Kv1.3 channels are involved in the activation and/or proliferation of many types of cells, including tumor cells (Bielanska et al., Curr. Cancer Drug Targets 9:904-14, 2009), microglia (Khanna et al., Am. J. Physiol. Cell Physiol. 280: C796-806, 2001) and differentiation of neuronal progenitor cells (Wang et al., J. Neurosci. 30:5020-7, 2010). Kv1.3 inhibitors may therefore be beneficial in the treatment of neuroinflammatory and neurodegenerative disorders, and cancers.

Kv1.3 is part of a sub-family of closely related potassium channels, designated Kv1.1 to Kv1.8. When dealing with large homologous families, it is always desirable for an inhibitor to be as selective and specific as possible for the desired target, to improve efficacy and safety, and avoid undesirable off-target effects. The most specific Kv1.3 inhibitors identified to date are venom peptides derived from various types of venomous organisms, such as snakes, arachnids (such as scorpions and spiders), sea anemones, etc. Such Kv1.3 inhibitors include the peptides ShK, Oskl, margatoxin and kaliotoxin, reviewed by Chandy et al., Trends in Pharmacol. Sci. 25:280-9, 2004. See also Abdel-Mottaleb et al., Toxicon 51:1424-30, 2008, and Mouhat et al., Biochem. J. 385(Pt 1):95-104, 2005.

Various attempts to engineer toxin peptides for particular properties, including specificity or potency, have been described, e.g. in WO2006/002850, WO2006/042151, WO2008/088422, WO2006/116156, WO2010/105184 and WO2014/116937.

### SUMMARY OF THE INVENTION

The invention relates to Kv1.3 inhibitors derived from a toxin peptide of the scorpion Parabuthus transvaalicus. The toxin peptide has the amino acid sequence QMDMRCSASVECKQKCLKAIGSIFGKCMNKKCKCYPR (SEQ ID NO 1).

Amongst other desirable properties, this molecule and derivatives or variants thereof have been found to be extremely selective inhibitors for the Kv1.3 potassium ion channel over other voltage-gated potassium channels, and typically also have high potency at blocking the Kv1.3 channel. Peptides that are highly selective for the Kv1.3 channel, such as those derived from PaT1, have the particular advantage of selectively targeting cells expressing Kv1.3 channels, such as specific populations of effector memory T cells. This selectivity thus gives Kv1.3 inhibitors derived from PaT1 the potential for targeted therapeutic effects.

Kv1.3 inhibitors based on the PaT1 toxin peptide have a short half-life in the body of a subject. For instance, Example 6 herein shows that such Kv1.3 inhibitors have a half-life of about 1 hour in a rat model organism. However, the present inventors have surprisingly found that, despite this short half-life, the Kv1.3 inhibitors described herein have a long-lasting effect on T cells. In particular, Example 8 herein shows that inflammation is reduced in a rat ear inflammation model up to 7 days after treatment with the Kv1.3 inhibitor. Additionally, Example 9 herein shows that treatment with a Kv1.3 inhibitor once every 5 days reduces inflammation in a rat model of arthritis. Without wishing to be bound by theory, it is hypothesised that the Kv1.3 inhibitor somehow, through its interaction with the Kv1.3 ion channel, “reprograms” T cells to remain inactive for an extended period, thereby suppressing inflammation for multiple days.

These surprising findings support the concept of treating a subject suffering from a disease, condition or disorder that may be treated using a Kv1.3 inhibitor as described herein following a regime of administering the inhibitor at roughly weekly intervals (i.e. once every 2 to 8 days). It is advantageous to increase as much as possible the period between administrations of an active compound as this saves time and effort (as fewer administrations need to be carried out over a given period) and expense (as less active compound needs to be used over a given period). Particularly in the case of active compounds administered by certain routes, such as subcutaneous injection, a longer interval between administrations also increases patient comfort and may thereby increase patient compliance with the treatment regime. For example, administering the Kv1.3 inhibitor in accordance with the present invention (i.e. once every 2 to 8 days) is superior to administering the inhibitor daily, for the above reasons. An unexpected finding described in the present disclosure is that administering a Kv1.3 inhibitor as described herein once every 2 to 8 days is possible despite their short in vivo half-life, as an effect of the inhibitor (such as reduced inflammation) is observed multiple days after administration.

Accordingly, the invention provides a Kv1.3 inhibitor, or a pharmaceutically acceptable salt thereof, for use in a method of treating or preventing a disease or disorder in a subject, wherein the Kv1.3 inhibitor comprises or consists of a peptide comprising or consisting of the sequence QMDMRCSASVECKQKCLKAIGSIFGKCMNKKCKCYPR (SEQ ID NO 1) or a variant thereof, wherein the variant (a) has at least 65% sequence identity to SEQ ID NO 1, and/or (b) differs from SEQ ID NO 1 by up to nine substitutions, insertions and/or deletions in total, and wherein the method comprises administering the Kv1.3 inhibitor to the subject once every 2 to 8 days.

### BRIEF DESCRIPTION OF FIGURES

FIG. 1 depicts the study design of keyhole limpet hemocyanin (KLH)-induced delayed type hypersensitivity (DTH) model to investigate the effect of a Kv1.3 inhibitor (peptide 100) on ear swelling after ear challenge on days 7, 9, 11 or 13. Ear edemas were measured at 24 hrs or 48 hrs post-challenge for respective ear challenge.

FIG. 2 shows the effect of peptide 100 administered at 300 nmol/kg on day 6 on ear swelling upon ear challenge on days 7, 9, 11 or 13, measured 24 hours post-challenge. Data are shown as single values and mean (n=8 rats/group). The mean values of vehicle and peptide 100 treated animals was compared for respective ear challenge time point by a two-sided unpaired t-test.

FIG. 3 shows the effect of peptide 100 administered at 300 nmol/kg on day 6 on ear swelling upon ear challenge on days 7, 9, 11 or 13, measured 48 hours post-challenge. Data are shown as single values and mean (n=8 rats/group). The mean values of vehicle and peptide 100 treated animals was compared for respective ear challenge time point by a two-sided unpaired t-test.

FIG. 4 shows the clinical score of the front paws in the rat collagen-induced arthritis (CIA) model described in Example 9.

FIG. 5 depicts the study design of keyhole limpet hemocyanin (KLH)-induced delayed type hypersensitivity (DTH) model to investigate the effect of different doses of a Kv1.3 inhibitor (peptide 100) on ear swelling after ear challenge on days 7 or 11. Ear edemas were measured at 24 hrs or 48 hrs post-challenge for respective ear challenge.

FIG. 6 shows the effect of peptide 100 administered at 10, 100, 300 or 700 nmol/kg on day 6 on ear swelling upon ear challenge on days 7 or 11, measured 24 hours post-challenge. Data are shown as single values and mean (n=8 rats/group). The mean values of vehicle and peptide 100 treated animals was compared for respective ear challenge time point by a two-sided unpaired t-test.

FIG. 7 shows the effect of peptide 100 administered at 10, 100, 300 or 700 nmol/kg on day 6 on ear swelling upon ear challenge on days 7 or 11, measured 48 hours post-challenge. Data are shown as single values and mean (n=8 rats/group). The mean values of vehicle and peptide 100 treated animals was compared for respective ear challenge time point by a two-sided unpaired t-test.

FIG. 8 shows the effect of peptide 100 administered at 1, 3, 10, 30 or 100 nmol/kg on day 6 on ear swelling upon ear challenge on days 7 or 11, measured 24 hours post-challenge. Data are shown as single values and mean (n=8 rats/group). The mean values of vehicle and peptide 100 treated animals was compared for respective ear challenge time point by a two-sided unpaired t-test.

FIG. 9 shows the effect of peptide 100 administered at 1, 3, 10, 30 or 100 nmol/kg on day 6 on ear swelling upon ear challenge on days 7 or 11, measured 48 hours post-challenge. Data are shown as single values and mean (n=8 rats/group). The mean values of vehicle and peptide 100 treated animals was compared for respective ear challenge time point by a two-sided unpaired t-test.

### DETAILED DESCRIPTION

The invention provides a Kv1.3 inhibitor, or a pharmaceutically acceptable salt thereof, for use in a method of treating or preventing a disease or disorder in a subject, wherein the Kv1.3 inhibitor comprises or consists of a peptide comprising or consisting of the sequence QMDMRCSASVECKQKCLKAIGSIFGKCMNKKCKCYPR (SEQ ID NO 1) or a variant thereof, wherein the variant (a) has at least 70% sequence identity to SEQ ID NO 1, and/or (b) differs from SEQ ID NO 1 by up to nine substitutions, insertions and/or deletions in total, and wherein the method comprises administering the Kv1.3 inhibitor to the subject once every 2 to 8 days.

The peptide may be any peptide described herein. The disease or disorder may be any disease or disorder described herein.

### Kv1.3 Inhibitor

The invention provides a Kv1.3 inhibitor, which comprises or consists of a peptide.

The term “Kv1.3 inhibitor” is used herein to denote a molecule or compound having inhibitor (or blocking) activity against the Kv1.3 ion channel, i.e. capable of inhibiting or eliminating ion flow through the Kv1.3 ion channel, such as by binding to the ion channel. The term “blocker” as used herein is synonymous with the term “inhibitor”. Thus, the Kv1.3 inhibitor of the invention may also be referred to as an “ion channel blocker” herein. The terms “blocker” and “inhibitor” should not be taken to imply any particular mechanism of action, or any particular mode of interaction with the ion channel itself.

The terms “Kv1.3 inhibitor” and “compound” are used interchangeably herein.

The term “Kv1.3” is used to refer to potassium voltage-gated channel subfamily A member 3, also referred to as KCNA3, HPCN3, HGK5, HuKIII and HLK3. “Subfamily A” may also be referred to as “shaker-related subfamily”. The human amino acid sequence of Kv1.3 is provided under UniProt accession number P22001, version P22001.3 (Q5VWN2).

The Kv1.3 channel is expressed on T and B lymphocytes and has been implicated in T cell activation. A number of groups are pursuing development of Kv1.3 blockers for the inhibition of immune responses as well as for various other indications. However, the Kv1.3 channel is part of a complex family of related ion channels, also including the Kv1.1, Kv1.2 and Kv1.6 channels, which have different physiological roles. Consequently, it is desirable for Kv1.3 inhibitors to be as selective as possible for Kv1.3 in preference to other ion channels, especially other voltage-gated potassium channels, such as Kv1.1, Kv1.2, Kv1.4, Kv1.5, Kv1.6, Kv1.7 and Kv1.8. More information on Kv1.3 and known inhibitors of Kv1.3 may be found in Murray et al. J Med Chem 2015, 58, 17, 6784-6802 and Tanner et al. Clin Immunol 2017, 180, 45-47.

The Kv1.3 inhibitor or pharmaceutically acceptable salt of the invention has Kv1.3 inhibitor activity. In other words, the Kv1.3 inhibitor of the invention (and the peptide component of the Kv1.3 inhibitor in isolation) has inhibitor or blocker activity at the Kv1.3 ion channel, i.e. it is capable of inhibiting ion flow through the Kv1.3 channel.

### IC₅₀ Values

IC₅₀ values may be used as a measure of inhibitor (or blocker) activity or potency. An IC₅₀ value is a measure of the concentration of an inhibitor required to achieve half of that compound's maximal inhibition of ion channel activity in a given assay. A compound which has a lower IC₅₀ at a particular ion channel than a reference compound can be considered to be a more active inhibitor, or a more potent inhibitor, than the reference compound. The terms “activity” and “potency” are used interchangeably.

IC₅₀ values may be determined using any appropriate assay, such as fluorescence-based assays measuring ion flux (e.g. thallium ion flux) and patch clamp assays, which may be performed as described in the Examples herein. Patch clamp assays may be preferred, e.g. using the QPatch® system.

In some embodiments, the Kv1.3 inhibitor of the invention has an IC₅₀ for human Kv1.3 potassium channel of about 400 nM or less, such as about 300 nM or less, such as about 200 nM or less, such as about 100 nM or less, such as about 50 nM or less, such as about 15 nM or less, such as about 10 nM or less, such as about 5 nM or less. Preferably the Kv1.3 inhibitor of the invention has an IC₅₀ of about 2 nM or less. More preferably Kv1.3 inhibitor of the invention has an IC₅₀ of about 1 nM or less, such as about 0.5 nM or less.

### Selectivity

The Kv1.3 inhibitors of the invention are selective for Kv1.3. In some embodiments, the Kv1.3 inhibitors of the invention are selective over Kv1.1, Kv1.2, Kv1.4, Kv1.5, Kv1.6, Kv1.7 and Kv1.8. In particular, the Kv1.3 inhibitors of the invention are selective for Kv1.3 over one or more of Kv1.1, Kv1.2 and Kv1.6.

For example, the Kv1.3 inhibitors of the invention may be selective for Kv1.3 over Kv1.1; selective for Kv1.3 over Kv1.2; selective for Kv1.3 over Kv1.6; selective for Kv1.3 over Kv1.1 and Kv1.2; selective for Kv1.3 over Kv1.1 and Kv1.6; selective for Kv1.3 over Kv1.2 and Kv1.6; or selective for Kv1.3 over Kv1.1, Kv1.2 and Kv1.6. Typically, the Kv1.3 inhibitors are selective for Kv1.3 over Kv1.1. They may additionally be selective for Kv1.3 over Kv1.2 and/or Kv1.6.

“Selective” in this context means that the Kv1.3 inhibitors have higher inhibitor activity against Kv1.3 than against the respective ones of Kv1.1, Kv1.2 and Kv1.6. Thus, their IC₅₀ against Kv1.3 is typically lower than against the respective other ion channel or channels.

Selectivity for Kv1.3 over another ion channel X may therefore be expressed as a ratio of the respective IC₅₀ values, e.g. as IC₅₀[X]/IC₅₀[Kv1.3].

The Kv1.3 inhibitors of the invention may have a selectivity for Kv1.3 over Kv1.1 of at least about 10, at least about 100, at least about 1000, or at least about 10000, and may be up to about 100000 or even higher. Typically, the Kv1.3 inhibitors of the invention have a selectivity for Kv1.3 over Kv1.1 of at least about 100, or at least about 1000.

The Kv1.3 inhibitors of the invention may have a selectivity for Kv1.3 over Kv1.2 of at least about 10, at least about 100, at least about 1000, or at least about 10000, and may be up to about 100000 or even higher. Typically, the inhibitors have a selectivity for Kv1.3 over Kv1.2 of at least 10, and preferably at least about 50 or at least about 100 or at least about 1000.

The Kv1.3 inhibitors of the invention may have a selectivity for Kv1.3 over Kv1.6 of at least about 10, at least about 100, at least about 1000, or at least about 10000, and may be up to about 100000 or even higher. Typically, the inhibitors have a selectivity for Kv1.3 over Kv1.6 of at least 100, or at least about 400, or at least about 1000.

The Kv1.3 inhibitors of the invention may have greater selectivity than known Kv1.3 inhibitors such as ShK, Mokatoxin (Moka1), Vm24, Odk2 or Osk1. Thus the Kv1.3 inhibitors of the invention may have higher selectivity for Kv1.3 over ion channel X, i.e. IC₅₀[X]/IC₅₀[Kv1.3], which is greater than the selectivity of the comparison molecule. The selectivity of the two Kv1.3 inhibitors will be determined under the same conditions for each ion channel to enable direct comparison. As mentioned above, any appropriate assays may be used, such as fluorescence-based ion flux assays and patch clamp assays.

The Kv1.3 inhibitors of the invention may have lower absolute inhibitor activity (i.e. higher IC₅₀) than known Kv1.3 inhibitors (such as Odk2 or Osk1) at any or all of Kv1.1, Kv1.2 and/or Kv1.6. However, it may be acceptable for the Kv1.3 inhibitors of the invention to have lower absolute inhibitor activity at any or all of these ion channels, as long as their selectivity for Kv1.3 is higher than that of the comparison compound. Typically, the Kv1.3 inhibitors of the invention combine high specificity for Kv1.3 with high potency.

### Pharmaceutically Acceptable Salts

A Kv1.3 inhibitor of the invention may be in the form of a pharmaceutically acceptable salt. All references herein to “a Kv1.3 inhibitor”, “a Kv1.3 inhibitor of the invention”, “peptide” or “peptide of the invention” should be considered to encompass any pharmaceutically acceptable salt thereof, regardless of whether “pharmaceutically acceptable salt” is explicitly recited. The Kv1.3 inhibitor may also be referred to as a “solvate” meaning a complex of defined stoichiometry formed between a solute (a Kv1.3 inhibitor or pharmaceutically acceptable salt thereof according to the invention) and a solvent. The solvent in this connection may, for example, be water, ethanol or another pharmaceutically acceptable organic species, such as a small-molecular organic species, such as, but not limited to, acetic acid or lactic acid. When the solvent in question is water, such a solvate is normally referred to as a hydrate. In some embodiments, the pharmaceutically acceptable salt of the invention is an acetate salt. In other words, the invention encompasses a salt comprising or consisting of cations of the Kv1.3 inhibitor and acetate anions. In some embodiments, the pharmaceutically acceptable salt of the invention is a chloride salt. In other words, the invention encompasses a salt comprising or consisting of cations of the Kv1.3 inhibitor and chloride anions.

### Peptide

The Kv1.3 inhibitor of the invention comprises or consists of a peptide comprising or consisting of the sequence QMDMRCSASVECKQKCLKAIGSIFGKCMNKKCKCYPR (SEQ ID NO 1) or a variant thereof, wherein the variant (a) has at least 70% sequence identity to SEQ ID NO 1, and/or (b) differs from SEQ ID NO 1 by up to nine substitutions, insertions and/or deletions in total.

SEQ ID NO 1 is the amino acid sequence of a toxin peptide of the scorpion Parabuthus transvaalicus. As described herein, this peptide is a selective Kv1.3 potassium ion channel inhibitor. Thus, it is the peptide part of the Kv1.3 inhibitor of the invention that is believed to inhibit Kv1.3. The peptide may be referred to herein as the “peptide component”, “peptide element”, “Kv1.3 inhibitor component” or “Kv1.3 inhibitor element” of the Kv1.3 inhibitor of the invention.

Variants of SEQ ID NO 1 are peptides comprising one or more amino acid that is different, additional or missing relative to the amino acids of SEQ ID NO 1. In other words, a variant comprises one or more amino acid changes compared to SEQ ID NO 1. Such variants may also be termed “derivatives”, “variant sequences”, “sequence variants” “variant peptides”, “peptide variants” or simply “peptides” herein.

In some embodiments, the Kv1.3 inhibitor of the invention comprises the peptide as described herein. In other words, in some embodiments the Kv1.3 inhibitor comprises the peptide along with other features or elements. In some embodiments, the Kv1.3 inhibitor of the invention consists of the peptide as described herein. In other words, in some embodiments the Kv1.3 inhibitor is the peptide (i.e. the Kv1.3 inhibitor consists of the peptide and no other features or elements).

In some embodiments, the peptide consists of the sequence QMDMRCSASVECKQKCLKAIGSIFGKCMNKKCKCYPR (SEQ ID NO 1) or a variant thereof, wherein the variant (a) has at least 70% sequence identity to SEQ ID NO 1, and/or (b) differs from SEQ ID NO 1 by up to nine substitutions, insertions and/or deletions in total.

### Amino Acids

Throughout the present description and claims the conventional three-letter and one-letter codes for naturally occurring amino acids are used, i.e.

A (Ala), G (Gly), L (Leu), I (Ile), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro); as well as generally accepted codes for other α-amino acids, such as sarcosine (Sar), norleucine (Nle), α-aminoisobutyric acid (Aib), 2,3-diaminopropanoic acid (Dap), 2,4-diaminobutanoic acid (Dab), 2,5-diaminopentanoic acid (ornithine; Orn), alpha-aminobutyric acid (Abu, also known as homo-alanine), hK, hLys or homo-Lys (homo-lysine), hQ, hGln or homo-Gln (homo-glutamine, also known as 6-oxolysine, L-5-carbamoylnorvaline, 6-amino-6-oxonorleucine or 5-(aminocarbonyl)norvaline), F(4-F) (4-fluoro-phenylalanine), F(4-NH₂) (4-amino-phenylalanine), F(4-NO₂) (4-nitro-phenylalanine), F(4-CH₃) (4-methyl-phenylalanine).

The designation [2-Amino-5-carboxypentanoyl] indicates a peptide residue of 2-amino-5-carboxypentanoic acid:

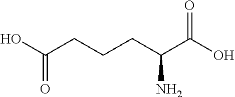 [SMILES: N[C@@H](CCCC(=O)O)C(=O)O]

which thus has a side chain similar to that of glutamic acid, but with an additional methylene group.

The designation [2,3-Diaminopropanoyl] indicates a peptide residue of 2,3-Diaminopropanoic acid, which has the following structure:

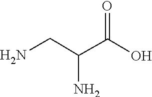

The designation [2,4-Diaminobutanoyl] indicates a peptide residue of 2,4-Diaminobutanoic acid, which has the following structure:

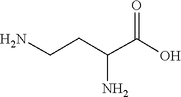

The designation [2-Amino-3-guanidinopropionyl] indicates a peptide residue of 2-Amino-3-guanidinopropionic acid, which has the following structure:

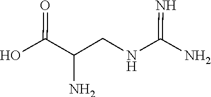

Such other α-amino acids may be shown in square brackets “[ ]” (e.g. “[Nle]”) when used in a general formula or sequence in the present specification, especially when the rest of the formula or sequence is shown using the single letter code. Unless otherwise specified, amino acid residues in peptides of the invention are of the L-configuration. However, D-configuration amino acids may be incorporated. In the present context, an amino acid code written with a small letter represents the D-configuration of said amino acid, e.g. “k” represents the D-configuration of lysine (K).

### Numbering of Amino Acid Positions

The amino acid residues of SEQ ID NO 1 are numbered from 1 to 37, in the conventional direction of N- to C-terminus. Throughout this specification, amino acid positions in variants of SEQ ID NO 1 are numbered according to the corresponding position in SEQ ID NO 1 when optimally aligned therewith. Thus, especially for variants which contain one or more insertions or deletions compared to SEQ ID NO 1, the numbering of any given residue reflects the corresponding residue in SEQ ID NO 1 and does not necessarily reflect its linear position in the sequence of the variant.

The residue present at a specific position may be indicated by the number of the relevant position alongside the single letter code or three letter code for the residue present. Thus, 1Q or Q1 (the two formats are interchangeable) indicates a glutamine (Q) residue at position 1, while 2Nle, 2[Nle], Nle2 or [Nle]2 indicates a norleucine residue at position 2.

An asterisk may be used to denote the position of a deletion relative to the sequence of SEQ ID NO 1. For example, “1*” indicates a deletion of the residue at position 1 as compared to SEQ ID NO 1.

An insertion may be indicated by a string of consecutive residues at a single position, e.g. “1QA” indicates an insertion of an alanine (A) residue after the glutamine (Q) residue at position 1.

In some embodiments, any substitutions compared to SEQ ID NO 1 are conservative substitutions. However, any substitution listed in any of the generic formulae provided below may be introduced at the respective position.

### Cysteines and Disulphide Bonds

The peptide contains six cysteine (C) residues which together form three disulphide bonds, between residues 6C and 27C, residues 12C and 32C, and residues 16C and 34C. Thus, in the Kv1.3 inhibitor or pharmaceutically acceptable salt of the invention, the peptide comprises a cysteine (C) at each of positions 6, 12, 16, 27, 32 and 34.

The disulphide bonds may be indicated graphically as follows by reference to SEQ ID NO 1:

QMDMRC(1)SASVEC(2)KQKC(3)LKAIGSIFGKC(1)MNKKC(2)
KC(3)YPR

where a pair of cysteine residues which participate together in a disulphide bond are indicated by the same numeral in parentheses. Similar notation can be applied to any of the other sequences in this application. Except where the context demands otherwise, it should be understood that an active inhibitor compound includes appropriate disulphide bonding. In some embodiments, the pattern of disulphide bridges is different to the pattern shown in SEQ ID NO 1 above. In such embodiments, the different pattern of disulphide bridges is indicated by the numbering system described above.

It may be desirable that no other cysteine residues are introduced into the variant of SEQ ID NO 1 by substitution or insertion. Thus, in some embodiments, the variant contains no other cysteine residues apart from those at positions corresponding to positions 6, 12, 16, 27, 32 and 34 of SEQ ID NO 1. In some embodiments, any substitutions or deletions in the variant of SEQ ID NO 1 are not at amino acid positions 6, 12, 16, 27, 32 and 34 of SEQ ID NO 1.

### Sequence Identity

In some embodiments, the variant has at least 70% sequence identity to SEQ ID NO 1.

The sequence of the variant of the Kv1.3 inhibitor of the invention may be expressed in terms of sequence identity instead of the number of substitutions, insertions and deletions relative to SEQ ID NO 1.

Accordingly, the invention provides a Kv1.3 inhibitor, or a pharmaceutically acceptable salt thereof, for use in a method of treating or preventing a disease or disorder in a subject, wherein the Kv1.3 inhibitor comprises or consists of a peptide comprising or consisting of the sequence QMDMRCSASVECKQKCLKAIGSIFGKCMNKKCKCYPR (SEQ ID NO 1) or a variant thereof, wherein the variant has at least 70% sequence identity to SEQ ID NO 1, and wherein the method comprises administering the Kv1.3 inhibitor to the subject once every 2 to 8 days.

In some embodiments, the peptide has at least 75% sequence identity to SEQ ID NO 1, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% identity to SEQ ID NO 1.

In some embodiments, “percentage (%) sequence identity” of the peptide is defined as the percentage of amino acids in the peptide sequence that are identical to the amino acids in the wild-type toxin peptide sequence SEQ ID NO 1 after aligning the sequences and introducing gaps, if necessary, to achieve the maximum percentage sequence identity, and not considering any conservative substitutions as part of the sequence identity. Sequence alignment can be carried out by the skilled person using techniques well known in the art, for example using publicly available software such as BLAST, BLAST2 or Align software. For examples, see Altschul et al., Methods in Enzymology 266: 460-480 (1996) or Pearson et al., Genomics 46: 24-36, 1997.

The percentage sequence identities used herein in the context of the present invention may be determined using these programs with their default settings. More generally, the skilled worker can readily determine appropriate parameters for determining alignment, including any algorithms needed to achieve maximal alignment over the full length of the sequences being compared.

### Substitutions, Insertions and Deletions

Variants of SEQ ID NO 1 differ from SEQ ID NO 1 in that one or more amino acid of SEQ ID NO 1 is deleted and/or one or more amino acid of SEQ ID NO 1 is substituted for a different amino acid and/or one or more amino acid is inserted into the sequence of SEQ ID NO 1. Amino acids may be inserted at an internal position of SEQ ID NO 1, at the N-terminus of SEQ ID NO 1 or at the C-terminus of SEQ ID NO 1. Thus the variants of SEQ ID NO 1 comprise one or more substitutions, insertions and/or deletions.

Except where otherwise noted, a “substitution” refers to the substitution (i.e. replacement) of a single amino acid in SEQ ID NO 1. Thus, for example, substitution of three contiguous amino acids in SEQ ID NO 1 constitutes three substitutions, rather than a single substitution. Likewise, “insertion” refers to insertion of a single amino acid into SEQ ID NO 1 (which may be internal, at the N-terminus, and/or at the C-terminus), so insertion of, for example, three contiguous amino acids in SEQ ID NO 1 constitutes three insertions, rather than a single insertion. “Deletion” refers to deletion of a single amino acid from SEQ ID NO 1, so deletion of, for example, three contiguous amino acids in SEQ ID NO 1 constitutes three deletions, rather than a single deletion.

In some embodiments, the variant differs from SEQ ID NO 1 by up to nine substitutions, insertions and/or deletions in total. The term “nine . . . in total” means that at most nine amino acids in total may be substituted in, and/or inserted into, and/or deleted from SEQ ID NO 1. In other words, the maximum combined total of all substitutions, insertions and deletions in the variant is nine, and within these nine there may be any combination of substitutions, insertions and/or deletions. Expressed another way, the variant comprises any combination of substitutions, insertions and/or deletion, to a maximum combined total of nine substitutions, insertions and deletions.

The sequence of the variant of the Kv1.3 inhibitor of the invention may be expressed in terms of the number of substitutions, insertions and deletions relative to SEQ ID NO 1 instead of in terms of sequence identity.

Accordingly, the invention provides a Kv1.3 inhibitor, or a pharmaceutically acceptable salt thereof, for use in a method of treating or preventing a disease or disorder in a subject, wherein the Kv1.3 inhibitor comprises or consists of a peptide comprising or consisting of the sequence QMDMRCSASVECKQKCLKAIGSIFGKCMNKKCKCYPR (SEQ ID NO 1) or a variant thereof, wherein the variant differs from SEQ ID NO 1 by up to nine substitutions, insertions and/or deletions in total, and wherein the method comprises administering the Kv1.3 inhibitor to the subject once every 2 to 8 days.

In some embodiments, the variant of SEQ ID NO 1 differs from SEQ ID NO 1 by up to eight substitutions, insertions and/or deletions in total. In some embodiments, the variant differs from SEQ ID NO 1 by up to seven, up to six, up to five, up to four, up to three or up to two substitutions, insertions and/or deletions in total or by one substitution, insertion or deletions in total. In some embodiments, the variant contains 9, 8, 7, 6, 5, 4, 3, 2 or 1 substitutions, insertions and/or deletions in total compared to SEQ ID NO 1. Preferably, the peptide comprises 6 substitutions, insertions and/or deletions in total compared to the sequence of SEQ ID NO 1.

### Amino Acids at Particular Positions

In some embodiments of the Kv1.3 inhibitor or pharmaceutically acceptable salt of the invention, the peptide comprises the following amino acids:
the amino acid at position 1 is H, N, P, p, Q, S, V or Y or is deleted;
the amino acid at position 2 is I, M or Ne or is deleted;
the amino acid at position 3 is D, E or S or is deleted;
the amino acid at position 4 is E, L, M, Nle, S or V or is deleted;
the amino acid at position 5 is R or K or is deleted;
the amino acid at position 7 is E, F, H, K, Orn, R, S, Y, 2,3-Diaminopropanoyl, 2,4-Diaminobutanoyl or 2-Amino-3-guanidinopropionyl;
the amino acid at position 8 is A, H, I, L, S or Y;
the amino acid at position 9 is F, L, P, S, Orn, V, Abu or 2,3-Diaminopropanoyl;
the amino acid at position 10 is K, P, Q, R or V;
the amino acid at position 11 is E or Q;
the amino acid at position 13 is A, E, G, K, L, Q or V;
the amino acid at position 14 is E, K, L, Q, V or 2-amino-5-carboxypentanoyl;
the amino acid at position 15 is K, L, P, S;
the amino acid at position 17 is K, L, R or Y or is deleted;
the amino acid at position 18 is A, D, G, K, Q, hQ, V or Y or is deleted;
the amino acid at position 19 is A, K, R or Y or is deleted;
the amino acid at position 20 is E, I, R or Y or is deleted;
the amino acid at position 21 is E, G, H or R;
the amino acid at position 22 is C, R or S;
the amino acid at position 23 is G, I, K, P or R;
the amino acid at position 26 is K or hK;
the amino acid at position 28 is M or Nle;
the amino acid at position 30 is G or K;
the amino acid at position 33 is H, K, R or V;
the amino acid at position 35 is Y, F(4-F), F(4-CH₃), F(4-NO₂) or F(4-NH₂);
the amino acid at position 36 is Q or P or is deleted; and/or
the amino acid at position 37 is C, G, R, S or (4-amino-5-hydroxypentyl)guanidine or is deleted.

In some embodiments of the Kv1.3 inhibitor or pharmaceutically acceptable salt of the invention, the peptide comprises the following amino acids:
the amino acid at position 1 is N or P or is deleted;
the amino acid at position 2 is M or Nle or is deleted;
the amino acid at position 3 is D or E or is deleted;
the amino acid at position 4 is M or Nle or is deleted;
the amino acid at position 5 is R, K or is deleted;
the amino acid at position 7 is S, 2,4-Diaminobutanoyl or 2-Amino-3-guanidinopropionyl;
the amino acid at position 14 is K or Q;
the amino acid at position 18 is K or A;
the amino acid at position 19 is K, S or A;
the amino acid at position 28 is M or Nle; and/or
the amino acid at position 37 is R or S.

In preferred embodiments of the Kv1.3 inhibitor or pharmaceutically acceptable salt of the invention, the peptide comprises the following amino acids:
the amino acid at position 1 is P;
the amino acid at position 2 is Nle;
the amino acid at position 3 is E;
the amino acid at position 4 is Nle;
the amino acid at position 18 is A; and/or
the amino acid at position 28 is Ne.

In preferred embodiments, positions 1-5 of the variant are deleted, or comprise or consist of an amino acid sequence selected from the following: QMDMR (SEQ ID NO: 155), NMDMR (SEQ ID NO: 156), P[Nle]D[Nle]R, and P[Nle]E[Nle]R.

In some embodiments, position 14 of the variant is K or Q. In some embodiments, position 28 of the variant is M or Ne. In some embodiments, position 37 of the variant is R or S. In some embodiments, the amino acid at position 1 of the variant of SEQ ID NO 1 is not Q. In some embodiments, the amino acid at position 1 of the variant of SEQ ID NO 1 is N or P or is deleted. In some embodiments, the variant of SEQ ID NO 1 comprises the amino acids 22S and 231.

In some embodiments of the Kv1.3 inhibitor of the invention, one or more positions of the variant of SEQ ID NO 1 are the same amino acid as the corresponding position in SEQ ID NO 1. In other words, the amino acid present in the relevant position(s) is the same amino acid present in SEQ ID NO 1 at the corresponding position(s).

In some embodiments, positions 6, 12, 16, 27, 32 and 34 of the variant are the same amino acid as the corresponding position in SEQ ID NO 1. In other words, in some embodiments the peptide comprises the following amino acids: 6C, 12C, 16C, 27C, 32C and 34C.

In some embodiments, positions 6, 12, 16, 24, 25, 27, 32 and 34 of the variant are the same amino acid as the corresponding position in SEQ ID NO 1. In other words, in some embodiments the peptide comprises the following amino acids: 6C, 12C, 16C, 24F, 25G, 27C, 32C and 34C.

In some embodiments, positions 6, 12, 16, 24, 25, 27, 29, 31, 32 and 34 of the variant are the same amino acid as the corresponding position in SEQ ID NO 1. In other words, in some embodiments the peptide comprises the following amino acids: 6C, 12C, 16C, 24F, 25G, 27C, 29N, 31K, 32C and 34C.

In some embodiments, the following positions of the variant are the same amino acid as the corresponding position in SEQ ID NO 1: positions 6, 8-13, 15-17, 20-27 and 29-36. In some embodiments, the following positions of the variant are the same amino acid as the corresponding position in SEQ ID NO 1: positions 6-13, 15-17, 20-27 and 29-36.

In some embodiments, any substitutions or deletions in the variant of SEQ ID NO 1 are at amino acid positions selected from positions 1-5, 7-11, 13-15, 17-23, 26, 28, 30, 33 and 35-37 of SEQ ID NO 1. Preferably, any substitutions or deletions in the variant of SEQ ID NO 1 are at amino acid positions selected from positions 1, 2, 3, 4, 5, 7, 14, 18, 19, 28 and 37 of SEQ ID NO 1.

In some embodiments, at least one amino acid in position 7, 8, 9, 10 or 11 of SEQ ID NO: 1 is substituted with an amino acid having a positively charged side chain and/or an amino acid having an aromatic side chain. In some embodiments, at least one amino acid in position 7, 8, 9 or 10 of SEQ ID NO: 1 is substituted with an amino acid having a positively charged side chain and/or an amino acid having an aromatic side chain. In some embodiments, exactly one amino acid in position 7, 8, 9 or 10 of SEQ ID NO: 1 is substituted with an amino acid having a positively charged side chain and/or an amino acid having an aromatic side chain. In some embodiments, the amino acid having a positively charged side chain is selected from H, R, Orn, 2,3-Diaminopropanoyl, 2-Amino-3-guanidinopropionyl and 2,4-Diaminobutanoyl. In some embodiments, the amino acid having an aromatic side chain is Y.

### Deletions

In some embodiments, if the variant of SEQ ID NO 1 contains one or more deletion, one of those deletions is at position 1. In some embodiments, if the variant of SEQ ID NO 1 contains two or more deletions, two of those deletions are at positions 1 and 2.

In some embodiments, variant comprises exactly 1, 2, 3, 4, 5, 6 or 7 deletions.

In some embodiments, the deletions in the peptide variant are selected from: deletion at position 1;
deletions at positions 1 and 2;
deletions at positions 1, 2 and 3;
deletions at positions 1, 2, 3 and 4;
deletions at positions 1, 2, 3, 4 and 5;
deletions at positions 1, 2, 3, 4, 5 and 36;
deletions at positions 1, 2, 3, 4, 5, 36 and 37;
deletion at position 17;
deletion at position 18;
deletion at position 19;
deletion at position 19;
deletion at position 20;
deletion at position 36; and
deletion at position 37.

In preferred embodiments, the deletions in the variant are at positions 1, 2, 3, 4 and 5.

### Insertions

In some embodiments, the variant comprises a maximum of four insertions compared to SEQ ID NO 1. In some embodiments, the variant comprises a maximum of three insertions, a maximum of two insertions or a maximum of one insertion compared to SEQ ID NO 1.

In some embodiments, the peptide comprises one or more insertions at the N-terminus (i.e. before position 1). In some embodiments, the peptide comprises one or more insertions only at the N-terminus. In some embodiments, the peptide comprises one or more insertions at the C-terminus (i.e. after position 37). In some embodiments, the peptide comprises one or more insertions only at the C-terminus. In some embodiments, the peptide comprises one or more insertions at both termini.

In some embodiments, the insertions at the N-terminus comprise or consist of the sequence GG or SG. In some embodiments, the insertions at the C-terminus comprise or consist of the sequence RRTA (SEQ ID NO: 158), HRRK (SEQ ID NO: 159), QSKA (SEQ ID NO: 160), AGPR (SEQ ID NO: 161), RSRT (SEQ ID NO: 162), RHKR (SEQ ID NO: 163), GGKR (SEQ ID NO: 164), PKTA (SEQ ID NO: 165), TDAR (SEQ ID NO: 166), HRQQ (SEQ ID NO: 167), RPRH (SEQ ID NO: 168), ARNA (SEQ ID NO: 169), TGRK (SEQ ID NO: 170), HERT (SEQ ID NO: 171), NTRT (SEQ ID NO: 172), QRNG (SEQ ID NO: 173), AHRN (SEQ ID NO: 174), PRSA (SEQ ID NO: 175), QRQS (SEQ ID NO: 176), QRRK (SEQ ID NO: 177), ARAK (SEQ ID NO: 178), AKRD (SEQ ID NO: 179), RDKT (SEQ ID NO: 180), RAKR (SEQ ID NO: 182), QRTR (SEQ ID NO: 183), ATRH (SEQ ID NO: 184), ARRS (SEQ ID NO: 185), AKTR (SEQ ID NO: 186), NRQR (SEQ ID NO: 187) or PRNT (SEQ ID NO: 188).

In some embodiments, the insertions in the variant are selected from:
GG at positions −1 and 0 (i.e. inserted before position 1);
SG at positions −1 and 0 (i.e. inserted before position 1);
R at position 38 (i.e. inserted after position 37);
Y at position 38 (i.e. inserted after position 37);
L at position 38 (i.e. inserted after position 37);
H at position 38 (i.e. inserted after position 37);
E at position 38 (i.e. inserted after position 37);
KS at positions 38 and 39 (i.e. inserted after position 37);
FE at positions 38 and 39 (i.e. inserted after position 37);
HR at positions 38 and 39 (i.e. inserted after position 37);
AK at positions 38 and 39 (i.e. inserted after position 37);
4-amino-5-hydroxypentanamide at position 38 (i.e. inserted after position 37);
ST at positions 38 and 39 (i.e. inserted after position 37);
RY at positions 38 and 39 (i.e. inserted after position 37);
RRTA (SEQ ID NO: 158) at positions 38-41 (i.e. inserted after position 37);
HRRK (SEQ ID NO: 159) at positions 38-41 (i.e. inserted after position 37); and
RRTK (SEQ ID NO: 157) at positions 38-41 (i.e. inserted after position 37).

In some embodiments, the peptide is a fusion protein comprising SEQ ID NO 1, or a variant thereof as defined herein, and one or more heterologous polypeptide sequences. In some embodiments, SEQ ID NO 1, or the variant thereof, is inserted within a heterologous scaffold polypeptide. In some embodiments, the peptide has a maximum length of 200 amino acids, 150 amino acids, 125 amino acids, 100 amino acids, 75 amino acids or 50 amino acids.

### Terminal Groups

A “H” (or “Hy-”) moiety at the N-terminus of a sequence indicates a hydrogen atom, corresponding to the presence of a free primary or secondary amino group at the N-terminus. Alternatively, a peptide may comprise an alternative N-terminal group (i.e. an N-terminal modification).

Thus, in some embodiments of the Kv1.3 inhibitor or pharmaceutically acceptable salt of the invention, the peptide comprises at the N-terminus a group selected from C₁₋₄ alkyl, acetyl (Ac), formyl, benzoyl and trifluoroacetyl.

An “—OH” moiety at the C-terminus of the sequence indicates the presence of a carboxy group (—COOH) at the C-terminus of the molecule. An “—NH₂” moiety at the C-terminus of the sequence indicates the presence of an amido (CONH₂) group at the C-terminus of the molecule. A “CH₂OH” moiety at the C-terminus indicates the presence of a hydroxyl group linked to an alkyl group at the C-terminus of the molecule. The CH₂OH moiety can be comprised in (4-amino-5-hydroxypentyl)guanidine or 4-amino-5-hydroxypentanamide.

Thus, in some embodiments of the Kv1.3 inhibitor or pharmaceutically acceptable salt of the invention, the peptide comprises at the C-terminus a carboxy group (—COOH), an amino group (—NH₂) or a hydroxymethyl group (—CH₂OH), preferably a carboxy group (—COOH) or an amino group (—NH₂).

### Sequences

In the context of an Kv1.3 inhibitor of the invention, the term “sequence” as used herein refers to the order of amino acids in the peptide of the Kv1.3 inhibitor. Specific sequences are referred to herein using a sequence identifier number (SEQ ID NO). Each reference herein to a SEQ ID NO refers to the sequence represented by that SEQ ID NO. A peptide that “comprises” a given sequence may include other amino acids at one or both ends of the sequence. A peptide that “consists of” a given sequence does not include any amino acids in its linear sequence other than those of the sequence, though it may comprise other features (e.g. chemical groups at the N-terminus or C-terminus).

In some embodiments of the Kv1.3 inhibitor or pharmaceutically acceptable salt of the invention, the peptide comprises or consists of one of the following sequences:

| SEQ ID | |
|---|---|
| NO | Sequence |
| 1 | QMDMRCSASVECKQKCLKAIGSIFGKCMNKKCKCYPR |
| 2 | QMDMRCSASVECKQKCLKAIGRGFGKCMNKKCKCYPR |
| 3 | NMDMRCSASVECKQKCLKAIGSIFGKCMNKKCKCYPR |
| 4 | NMDMRCSASVECKQKCLKAIGRGFGKCMNKKCKCYPR |
| 5 | MDMRCSASVECKQKCLKAIGSIFGKCMNKKCKCYPR |
| 6 | DMRCSASVECKQKCLKAIGSIFGKCMNKKCKCYPR |
| 7 | P[Nle]E[Nle]RCYASVECKQKCLAAIGSIFGKC[Nle]NKKCKCYPR |
| 8 | NIDMRCSASVECKQKCLKAIGSIFGKCMNKKCKCYPR |
| 9 | NMDVRCSASVECKQKCLKAIGSIFGKCMNKKCKCYPR |
| 10 | NMDMRCSASVECKQKCKDAIGSIFGKCMNKKCKCYPR |
| 11 | GGNMDMRCSASVECKQKCLKAIGSIFGKCMNKKCKCYPR |
| 12 | NMEMRCSASVECKQKCLKAIGSIFGKCMNKKCKCYPR |
| 13 | SGNMDMRCSASVECKQKCLKAIGSIFGKCMNKKCKCYPR |
| 14 | N[Nle]DMRCSASVECKQKCLKAIGSIFGKCMNKKCKCYPR |
| 15 | NMD[Nle]RCSASVECKQKCLKAIGSIFGKCMNKKCKCYPR |
| 16 | NMDMRCSASVECKQKCLKAIGSIFGKC[Nle]NKKCKCYPR |
| 17 | NMDMRCSASVECKVKCLKAIGSIFGKCMNKKCKCYPR |
| 18 | NMDMRCSASVECKQLCLKAIGSIFGKCMNKKCKCYPR |
| 19 | NMDMRCSASVECKQKCKKAIGSIFGKCMNKKCKCYPR |
| 20 | NMDMRCSASVECKQKCLDAIGSIFGKCMNKKCKCYPR |
| 21 | NMDMRCSASVECKQKCLKAIRSIFGKCMNKKCKCYPR |
| 22 | NMDMRCSASVECKQKCLKAIESIFGKCMNKKCKCYPR |
| 23 | NMDMRCSASVECKQKCLKAIGSIFGKCMNKKCKCYPRRRTA |
| 24 | NMDMRCSASVECKQKCLKAIGSIFGKCMNKKCKCYPRHRRK |
| 25 | NMDMRCKASVECKQKCLKAIGSIFGKCMNKKCKCYPR |
| 26 | NMDMRCSISVECKQKCLKAIGSIFGKCMNKKCKCYPR |
| 27 | NMDMRCSASRECKQKCLKAIGSIFGKCMNKKCKCYPR |
| 28 | NMDMRCSASVQCKQKCLKAIGSIFGKCMNKKCKCYPR |
| 29 | NMDMRCSASVECLQKCLKAIGSIFGKCMNKKCKCYPR |
| 30 | NMDMRCSASVECAQKCLKAIGSIFGKCMNKKCKCYPR |
| 31 | NMDMRCSASVECKEKCLKAIGSIFGKCMNKKCKCYPR |
| 32 | NMDMRCSASVECKLKCLKAIGSIFGKCMNKKCKCYPR |
| 33 | NMDMRCSASVECKQKCLKAIHSIFGKCMNKKCKCYPR |
| 34 | NMDMRCSASVECKQKCLKAIGSKFGKCMNKKCKCYPR |
| 35 | NMDMRCSASVECKQKCLKAIGSRFGKCMNKKCKCYPR |
| 36 | NMDMRCSASVECKQKCLKAIGSIFGKCMNGKCKCYPR |
| 37 | NMDMRCSASVECKQKCLKAIGSIFGKCMNKKCHCYPR |
| 38 | NMDMRCSASVECKQKCLKAIGSIFGKCMNKKCVCYPR |
| 39 | N[Nle]D[Nle]RCSASVECKQKCLKAIGSIFGKCMNKKCKCYPR |
| 40 | N[Nle]D[Nle]RCSASVECKQKCLKAIGSIFGKC[Nle]NKKCKCYPR |
| 41 | P[Nle]D[Nle]RCSASVECKQKCLKAIGSIFGKC[Nle]NKKCKCYPR |
| 42 | N[Nle]D[Nle]RCRASVECKQKCLKAIGSIFGKC[Nle]NKKCKCYPR |
| 43 | N[Nle]D[Nle]RCSASVECEQKCLKAIGSIFGKC[Nle]NKKCKCYPR |
| 44 | N[Nle]D[Nle]RCSASVECQQKCLKAIGSIFGKC[Nle]NKKCKCYPR |
| 45 | N[Nle]D[Nle]RCSASVECKKKCLKAIGSIFGKC[Nle]NKKCKCYPR |
| 46 | N[Nle]S[Nle]RCSASVECKQKCLKAIGSIFGKC[Nle]NKKCKCYPR |
| 47 | N[Nle]D[Nle]RCSHSVECKQKCLKAIGSIFGKC[Nle]NKKCKCYPR |
| 48 | N[Nle]D[Nle]RCSASVECKQSCLKAIGSIFGKC[Nle]NKKCKCYPR |
| 49 | N[Nle]D[Nle]RCSASVECKQKCKAIGSIFGKC[Nle]NKKCKCYPR |
| 50 | NMDMRCSASVECKQKCYKAIGSIFGKCMNKKCKCYPR |
| 51 | NMDMRCSASVECKQKCRKAIGSIFGKCMNKKCKCYPR |
| 52 | NMDMRCSASVECKQKCLAAIGSIFGKCMNKKCKCYPR |
| 53 | NMDMRCSASVECKQKCLYAIGSIFGKCMNKKCKCYPR |
| 54 | NMDMRCSASVECKQKCLAIGSIFGKCMNKKCKCYPR |
| 55 | NMDMRCSASVECKQKCLKYIGSIFGKCMNKKCKCYPR |
| 56 | NMDMRCSASVECKQKCLKRIGSIFGKCMNKKCKCYPR |
| 57 | NMDMRCSASVECKQKCLKIGSIFGKCMNKKCKCYPR |
| 58 | NMDMRCSASVECKQKCLKAYGSIFGKCMNKKCKCYPR |
| 59 | NMDMRCSASVECKQKCLKAEGSIFGKCMNKKCKCYPR |
| 60 | NMDMRCSASVECKQKCLKARGSIFGKCMNKKCKCYPR |
| 61 | NMDMRCSASVECKQKCLKAGSIFGKCMNKKCKCYPR |
| 62 | N[Nle]D[Nle]RCSASVECKQKCLKAIGSPFGKC[Nle]NKKCKCYPR |
| 63 | N[Nle]D[Nle]RCSASKECKQKCLKAIGSIFGKC[Nle]NKKCKCYPR |
| 64 | H[Nle]D[Nle]RCSASVECKQKCLKAIGSIFGKC[Nle]NKKCKCYPR |
| 65 | Y[Nle]D[Nle]RCSASVECKQKCLKAIGSIFGKC[Nle]NKKCKCYPR |
| 66 | S[Nle]D[Nle]RCSASVECKQKCLKAIGSIFGKC[Nle]NKKCKCYPR |
| 67 | V[Nle]D[Nle]RCSASVECKQKCLKAIGSIFGKC[Nle]NKKCKCYPR |
| 68 | S[Nle]D[Nle]RCSA[Abu]VECKQKCLKAIGSIFGKCMNKKCKCYPR |
| 69 | S[Nle]D[Nle]RCSASVECKQKCLKAIGSIFG[homo-Lys]CMNKKCKCYPR |
| 70 | S[Nle]D[Nle]RCSASVECKQKCLKAIGSIFGKCMNKKCKC[F(4-NH2)]PR |
| 71 | S[Nle]D[Nle]RCSASVECGQKCLKAIGSIFGKCMNKKCKCYPR |
| 72 | S[Nle]D[Nle]RCSASVECVQKCLKAIGSIFGKCMNKKCKCYPR |
| 73 | S[Nle]D[Nle]RCSASVECK[2-Amino-5- |
| | carboxypentanoyl]KCLKAIGSIFGKCMNKKCKCYPR |
| 74 | S[Nle]D[Nle]RCSASVECKQKCLKAIGSIFGKCMNKKCKCYQ |
| 75 | S[Nle]D[Nle]RCSASVECKQKCLKAIGSIFGKCMNKKCRCYPR |
| 76 | S[Nle]DERCSASVECKQKCLKAIGSIFGKCMNKKCKCYPR |
| 77 | S[Nle]D[Nle]RCSASVECKQKCLGAIGSIFGKCMNKKCKCYPR |
| 78 | S[Nle]D[Nle]RCSASVECKQKCLVAIGSIFGKCMNKKCKCYPR |
| 79 | S[Nle]D[Nle]RCSASVECAQSCLKAIGSIFGKCMNKKCKCYPR |
| 80 | S[Nle]D[Nle]RCSASVECAQKCLAAIGSIFGKCMNKKCKCYPR |
| 81 | S[Nle]D[Nle]RCSASVECAQLCLAAIGSIFGKCMNKKCKCYPR |
| 82 | P[Nle]E[Nle]RC[2,4- |
| | Diaminobutanoyl]ASVECKQKCLAAIGSIFGKC[Nle]NKKCKCYPR |
| 83 | P[Nle]D[Nle]RCSASVECKQKCLKAIGCIFGKC[Nle]NKKCKCYPC |
| 84 | S[Nle]D[Nle]RCSASVECKEKCLQAIGSIFGKC[Nle]NKKCKCYPR |
| 85 | P[Nle]D[Nle]RCSASVECKEKCL[homo-Gln]AIGSIFGKC[Nle]NKKCKCYPR |
| 86 | CSASVECKQKCLKAIGCIFGKC[Nle]NKKCKCYPC |
| 87 | S[Nle]D[Nle]RCSASVECKQKCLAAIGCIFGKC[Nle]NKKCKCYPC |
| 88 | P[Nle]E[Nle]RC[2-Amino-3- |
| | guanidinopropionyl]ASVECKQKCLAAIGSIFGKC[Nle]NKKCKCYPR |
| 89 | S[Nle]D[Nle]RCSALVECKQKCLKAIGSIFGKCMNKKCKCYPR |
| 90 | S[Nle]D[Nle]RCSAVVECKQKCLKAIGSIFGKCMNKKCKCYPR |
| 91 | S[Nle]D[Nle]RCSASVECKQKCLKAIGSIFGKCMNKKCKC[F(4-F)]PR |
| 92 | S[Nle]D[Nle]RCSASVECKQKCLKAIGSIFGKCMNKKCKC[F(4-NO₂)]PR |
| 93 | S[Nle]D[Nle]RCSASVECKQKCLKAIGSIFGKCMNKKCKC[F(4-CH₃)]PR |
| 94 | [Nle]D[Nle]RCSASVECKQKCLKAIGSIFGKC[Nle]NKKCKCYPR |
| 95 | NID[Nle]RCSASVECKQKCLKAIGSIFGKC[Nle]NKKCKCYPR |
| 96 | PIE[Nle]RCSASVECKQKCLKAIGSIFGKC[Nle]NKKCKCYPR |
| 97 | P[Nle]E[Nle]RCSASVECKQKCLAAIGSIFGKC[Nle]NKKCKCYPR |
| 98 | P[Nle]E[Nle]RCSA[Orn]VECKQKCLAAIGSIFGKC[Nle]NKKCKCYPR |
| 99 | P[Nle]E[Nle]RCSASVECKQKCLLAIGSIFGKC[Nle]NKKCKCYPR |
| 100 | PIDERCSASVECKQKCLAAIGSIFGKC[Nle]NKKCKCYPR |
| 101 | PIE[Nle]RCSASVECKQKCLAAIGSIFGKC[Nle]NKKCKCYPR |
| 102 | P[Nle]D[Nle]RCSASVECAQKCLAAIGSIFGKCMNKKCKCYPR |
| 103 | P[Nle]E[Nle]RCSASVECAQKCLAAIGSIFGKC[Nle]NKKCKCYPR |
| 104 | CSASVECKQKCLKAIGSIFGKC[Nle]NKKCKCYPR |
| 105 | P[Nle]E[Nle]RC(1)[2,3- |
| | Diaminopropanoyl]ASVEC(2)KQKC(3)LAAIGSIFGKC(1)[Nle]NKKC(2)KC(3) |
| | YPR |
| 106 | RCSASVECKQKCLKAIGSIFGKC[Nle]NKKCKCYPR |
| 107 | SKCSASVECKQKCLKAIGSIFGKC[Nle]NKKCKCYPR |
| 108 | LRCSASVECKQKCLKAIGSIFGKC[Nle]NKKCKCYPR |
| 109 | CSASVECKQKCLKAIGSIFGKC[Nle]NKKCKCYPS |
| 110 | CSASVECKQKCLKAIGSIFGKC[Nle]NKKCKCYS |
| 111 | CSASVECKQKCLKAIGSIFGKC[Nle]NKKCKCYG |
| 112 | CSASVECKQKCLKAIGSIFGKC[Nle]NKKCKCY |
| 113 | P[Nle]E[Nle]RC[Orn]ASVECKQKCLAAIGSIFGKC[Nle]NKKCKCYPR |
| 114 | P[Nle]E[Nle]RCHASVECKQKCLAAIGSIFGKC[Nle]NKKCKCYPR |
| 115 | LRCSASVECKQKCLAAIGSIFGKC[Nle]NKKCKCYPR |
| 116 | LRCSASVECKQKCLAAIGSIFGKCMNKKCKCYPR |
| 117 | CSASVECKQKCLKAIGSIFGKCMNKKCKCYPR |
| 118 | P[Nle]E[Nle]RCSASVECKEKCLAAIGSIFGKC[Nle]NKKCKCYPR |
| 119 | p[Nle]E[Nle]RCSASVECKQKCLAAIGSIFGKC[Nle]NKKCKCYPR |
| 120 | P[Nle]E[Nle]RCSASVECKQKCLAAIGSIFGKC[Nle]NKKCKCYR |
| 121 | [Nle]E[Nle]RCSASVECKQKCLAAIGSIFGKC[Nle]NKKCKCYPR |
| 122 | P[Nle]E[Nle]RCSASVECKQKCLAAIGSIFGKC[Nle]NKKCKCYPRR |
| 123 | P[Nle]E[Nle]RCSASVECKQKCLAAIGSIFGKC[Nle]NKKCKCYPRY |
| 124 | P[Nle]E[Nle]RCSASVECKQKCLAAIGSIFGKC[Nle]NKKCKCYPRL |
| 125 | P[Nle]E[Nle]RCSASVECKQKCLAAIGSIFGKC[Nle]NKKCKCYPRH |
| 126 | P[Nle]E[Nle]RCSASVECKQKCLAAIGSIFGKC[Nle]NKKCKCYPRE |
| 127 | P[Nle]E[Nle]RCSASVECKQKCLAAIGSIFGKC[Nle]NKKCKCYPRKS |
| 128 | P[Nle]E[Nle]RCSASVECKQKCLAAIGSIFGKC[Nle]NKKCKCYPRFE |
| 129 | P[Nle]E[Nle]RCSASVECKQKCLAAIGSIFGKC[Nle]NKKCKCYPRHR |
| 130 | P[Nle]E[Nle]RCSASVECKQKCLAAIGSIFGKC[Nle]NKKCKCYPRAK |
| 131 | P[Nle]E[Nle]RCSASVECKQKCLAAIGSIFGKC[Nle]NKKCKCYP-[(4-amino-5- |
| | hydroxypentyl)guanidine] |
| 132 | P[Nle]E[Nle]RCSASVECKQKCLAAIGSIFGKC[Nle]NKKCKCYPR-[4-amino-5- |
| | hydroxypentanamide] |
| 133 | P[Nle]E[Nle]RCSASVECKQKCLAAIGSIFGKC[Nle]NKKCKCYPRST |
| 134 | P[Nle]E[Nle]RCSASVECKQKCLAAIGSIFGKC[Nle]NKKCKCYPRRY |
| 135 | P[Nle]E[Nle]RCSSSVECKQKCLAAIGSIFGKC[Nle]NKKCKCYPR |
| 136 | P[Nle]E[Nle]RCSLSVECKQKCLAAIGSIFGKC[Nle]NKKCKCYPR |
| 137 | P[Nle]E[Nle]RCSAPVECKQKCLAAIGSIFGKC[Nle]NKKCKCYPR |
| 138 | P[Nle]E[Nle]RCSASPECKQKCLAAIGSIFGKC[Nle]NKKCKCYPR |
| 139 | P[Nle]E[Nle]RCSASQECKQKCLAAIGSIFGKC[Nle]NKKCKCYPR |
| 140 | P[Nle]E[Nle]RCSASVECLQKCLAAIGSIFGKC[Nle]NKKCKCYPR |
| 141 | P[Nle]E[Nle]RCSASVECKQPCLAAIGSIFGKC[Nle]NKKCKCYPR |
| 142 | P[Nle]E[Nle]RCEASVECKQKCLAAIGSIFGKC[Nle]NKKCKCYPR |
| 143 | P[Nle]E[Nle]RCFASVECKQKCLAAIGSIFGKC[Nle]NKKCKCYPR |
| 144 | P[Nle]E[Nle]RCSYSVECKQKCLAAIGSIFGKC[Nle]NKKCKCYPR |
| 145 | P[Nle]E[Nle]RCSAFVECKQKCLAAIGSIFGKC[Nle]NKKCKCYPR |
| 146 | P[Nle]E[Nle]RCSA[2,3- |
| | Diaminopropanoyl]VECKQKCLAAIGSIFGKC[Nle]NKKCKCYPR |
| 147 | P[Nle]E[Nle]RC[2,4- |
| | Diaminobutanoyl]ASVECAQSCLAAIGSIFGKC[Nle]NGKCKCYPR |
| 148 | P[Nle]E[Nle]RCRASVECAQSCLAAIGSIFGKC[Nle]NGKCKCYPR |
| 149 | P[Nle]E[Nle]RCSASRECAQSCLAAIGSIFGKC[Nle]NGKCKCYPR |
| 150 | P[Nle]E[Nle]RCSASVECKQKCLAKIGSIFGKC[Nle]NKKCKCYPR |

In some embodiments, the Kv1.3 inhibitor or pharmaceutical salt of the invention comprises a peptide consisting of the sequence of any one of SEQ ID NOs 1 to 150. In some embodiments, the Kv1.3 inhibitor or pharmaceutical salt of the invention consists of a peptide consisting of the sequence of any one of SEQ ID NOs 1 to 150. In preferred embodiments, the Kv1.3 inhibitor or pharmaceutical salt of the invention comprises a peptide consisting of the sequence of any one of SEQ ID NO 97. In preferred embodiments, the Kv1.3 inhibitor or pharmaceutical salt of the invention consists of a peptide consisting of the sequence of any one of SEQ ID NO 97.

### Peptides

In the context of an Kv1.3 inhibitor of the invention, the term “peptide” as used herein refers to the peptide component of the Kv1.3 inhibitor. The term peptide encompasses additional features beyond the sequence (i.e. order of amino acids) of the peptide, namely the chemical groups at the N-terminus and C-terminus of the peptide and the pattern of disulphide bridges in the peptide. For ease of reference, specific peptides are herein assigned a peptide number (Ptd no.). Each reference herein to a peptide number refers to the peptide represented by that peptide number. A Kv1.3 inhibitor that “comprises” a given peptide may include other features. A Kv1.3 inhibitor that “consists of” a given peptide includes only the features of that peptide.

In some embodiments of the Kv1.3 inhibitor or pharmaceutically acceptable salt of the invention, the peptide is selected from the following peptides:

| SEQ | | |
|---|---|---|
| ID | Ptd | |
| NO | No. | Peptide |
| 1 | 1 | H-QMDMRC(1)SASVEC(2)KQKC(3)LKAIGSIFGKC(1)MNKKC(2)KC(3)YPR-OH |
| 2 | 2 | H-QMDMRC(1)SASVEC(2)KQKC(3)LKAIGRGFGKC(1)MNKKC(2)KC(3)YPR-OH |
| 3 | 3 | H-NMDMRC(1)SASVEC(2)KQKC(3)LKAIGSIFGKC(1)MNKKC(2)KC(3)YPR-OH |
| 3 | 4 | H-NMDMRC(1)SASVEC(2)KQKC(3)LKAIGSIFGKC(1)MNKKC(2)KC(3)YPR-NH₂ |
| 4 | 5 | H-NMDMRC(1)SASVEC(2)KQKC(3)LKAIGRGFGKC(1)MNKKC(2)KC(3)YPR-OH |
| 5 | 6 | H-MDMRC(1)SASVEC(2)KQKC(3)LKAIGSIFGKC(1)MNKKC(2)KC(3)YPR-OH |
| 6 | 7 | H-DMRC(1)SASVEC(2)KQKC(3)LKAIGSIFGKC(1)MNKKC(2)KC(3)YPR-OH |
| 7 | 8 | H-P[Nle]E[Nle]RC(1)YASVEC(2)KQKC(3)LAAIGSIFGKC(1)[Nle]NKKC(2)KC(3)YPR- |
| | | OH |
| 8 | 9 | H-NIDMRC(1)SASVEC(2)KQKC(3)LKAIGSIFGKC(1)MNKKC(2)KC(3)YPR-OH |
| 9 | 10 | H-NMDVRC(1)SASVEC(2)KQKC(3)LKAIGSIFGKC(1)MNKKC(2)KC(3)YPR-OH |
| 10 | 11 | H-NMDMRC(1)SASVEC(2)KQKC(3)KDAIGSIFGKC(1)MNKKC(2)KC(3)YPR-OH |
| 11 | 12 | H-GGNMDMRC(1)SASVEC(2)KQKC(3)LKAIGSIFGKC(1)MNKKC(2)KC(3)YPR-OH |
| 12 | 13 | H-NMEMRC(1)SASVEC(2)KQKC(3)LKAIGSIFGKC(1)MNKKC(2)KC(3)YPR-OH |
| 13 | 14 | H-SGNMDMRC(1)SASVEC(2)KQKC(3)LKAIGSIFGKC(1)MNKKC(2)KC(3)YPR-OH |
| 14 | 15 | H-N[Nle]DMRC(1)SASVEC(2)KQKC(3)LKAIGSIFGKC(1)MNKKC(2)KC(3)YPR-NH₂ |
| 15 | 16 | H-NMD[Nle]RC(1)SASVEC(2)KQKC(3)LKAIGSIFGKC(1)MNKKC(2)KC(3)YPR-NH₂ |
| 16 | 17 | H-NMDMRC(1)SASVEC(2)KQKC(3)LKAIGSIFGKC(1)[Nle]NKKC(2)KC(3)YPR-NH₂ |
| 17 | 18 | H-NMDMRC(1)SASVEC(2)KVKC(3)LKAIGSIFGKC(1)MNKKC(2)KC(3)YPR-NH₂ |
| 18 | 19 | H-NMDMRC(1)SASVEC(2)KQLC(3)LKAIGSIFGKC(1)MNKKC(2)KC(3)YPR-NH₂ |
| 19 | 20 | H-NMDMRC(1)SASVEC(2)KQKC(3)KKAIGSIFGKC(1)MNKKC(2)KC(3)YPR-NH₂ |
| 20 | 21 | H-NMDMRC(1)SASVEC(2)KQKC(3)LDAIGSIFGKC(1)MNKKC(2)KC(3)YPR-NH₂ |
| 21 | 22 | H-NMDMRC(1)SASVEC(2)KQKC(3)LKAIRSIFGKC(1)MNKKC(2)KC(3)YPR-NH₂ |
| 22 | 23 | H-NMDMRC(1)SASVEC(2)KQKC(3)LKAIESIFGKC(1)MNKKC(2)KC(3)YPR-NH₂ |
| 23 | 24 | H-NMDMRC(1)SASVEC(2)KQKC(3)LKAIGSIFGKC(1)MNKKC(2)KC(3)YPRRRTA- |
| | | NH₂ |
| 24 | 25 | H-NMDMRC(1)SASVEC(2)KQKC(3)LKAIGSIFGKC(1)MNKKC(2)KC(3)YPRHRRK- |
| | | NH₂ |
| 25 | 26 | H-NMDMRC(1)KASVEC(2)KQKC(3)LKAIGSIFGKC(1)MNKKC(2)KC(3)YPR-NH₂ |
| 26 | 27 | H-NMDMRC(1)SISVEC(2)KQKC(3)LKAIGSIFGKC(1)MNKKC(2)KC(3)YPR-NH₂ |
| 27 | 28 | H-NMDMRC(1)SASREC(2)KQKC(3)LKAIGSIFGKC(1)MNKKC(2)KC(3)YPR-NH₂ |
| 28 | 29 | H-NMDMRC(1)SASVQC(2)KQKC(3)LKAIGSIFGKC(1)MNKKC(2)KC(3)YPR-NH₂ |
| 29 | 30 | H-NMDMRC(1)SASVEC(2)LQKC(3)LKAIGSIFGKC(1)MNKKC(2)KC(3)YPR-NH₂ |
| 30 | 31 | H-NMDMRC(1)SASVEC(2)AQKC(3)LKAIGSIFGKC(1)MNKKC(2)KC(3)YPR-NH₂ |
| 31 | 32 | H-NMDMRC(1)SASVEC(2)KEKC(3)LKAIGSIFGKC(1)MNKKC(2)KC(3)YPR-NH₂ |
| 32 | 33 | H-NMDMRC(1)SASVEC(2)KLKC(3)LKAIGSIFGKC(1)MNKKC(2)KC(3)YPR-NH₂ |
| 33 | 34 | H-NMDMRC(1)SASVEC(2)KQKC(3)LKAIHSIFGKC(1)MNKKC(2)KC(3)YPR-NH₂ |
| 34 | 35 | H-NMDMRC(1)SASVEC(2)KQKC(3)LKAIGSKFGKC(1)MNKKC(2)KC(3)YPR-NH₂ |
| 35 | 36 | H-NMDMRC(1)SASVEC(2)KQKC(3)LKAIGSRFGKC(1)MNKKC(2)KC(3)YPR-NH₂ |
| 36 | 37 | H-NMDMRC(1)SASVEC(2)KQKC(3)LKAIGSIFGKC(1)MNGKC(2)KC(3)YPR-NH₂ |
| 37 | 38 | H-NMDMRC(1)SASVEC(2)KQKC(3)LKAIGSIFGKC(1)MNKKC(2)HC(3)YPR-NH₂ |
| 38 | 39 | H-NMDMRC(1)SASVEC(2)KQKC(3)LKAIGSIFGKC(1)MNKKC(2)VC(3)YPR-NH₂ |
| 39 | 40 | H-N[Nle]D[Nle]RC(1)SASVEC(2)KQKC(3)LKAIGSIFGKC(1)MNKKC(2)KC(3)YPR- |
| | | NH₂ |
| 40 | 41 | H-N[Nle]D[Nle]RC(1)SASVEC(2)KQKC(3)LKAIGSIFGKC(1)[Nle]NKKC(2)KC(3)YPR- |
| | | NH₂ |
| 41 | 42 | H-P[Nle]D[Nle]RC(1)SASVEC(2)KQKC(3)LKAIGSIFGKC(1)[Nle]NKKC(2)KC(3)YPR- |
| | | NH₂ |
| 42 | 43 | H-N[Nle]D[Nle]RC(1)RASVEC(2)KQKC(3)LKAIGSIFGKC(1)[Nle]NKKC(2)KC(3)YPR- |
| | | NH₂ |
| 43 | 44 | H-N[Nle]D[Nle]RC(1)SASVEC(2)EQKC(3)LKAIGSIFGKC(1)[Nle]NKKC(2)KC(3)YPR- |
| | | NH₂ |
| 44 | 45 | H-N[Nle]D[Nle]RC(1)SASVEC(2)QQKC(3)LKAIGSIFGKC(1)[Nle]NKKC(2)KC(3)YPR- |
| | | NH₂ |
| 45 | 46 | H-N[Nle]D[Nle]RC(1)SASVEC(2)KKKC(3)LKAIGSIFGKC(1)[Nle]NKKC(2)KC(3)YPR- |
| | | NH₂ |
| 46 | 47 | H-N[Nle]S[Nle]RC(1)SASVEC(2)KQKC(3)LKAIGSIFGKC(1)[Nle]NKKC(2)KC(3)YPR- |
| | | NH₂ |
| 47 | 48 | H-N[Nle]D[Nle]RC(1)SHSVEC(2)KQKC(3)LKAIGSIFGKC(1)[Nle]NKKC(2)KC(3)YPR- |
| | | NH₂ |
| 48 | 49 | H-N[Nle]D[Nle]RC(1)SASVEC(2)KQSC(3)LKAIGSIFGKC(1)[Nle]NKKC(2)KC(3)YPR- |
| | | NH₂ |
| 49 | 50 | H-N[Nle]D[Nle]RC(1)SASVEC(2)KQKC(3)KAIGSIFGKC(1)[Nle]NKKC(2)KC(3)YPR- |
| | | NH₂ |
| 50 | 51 | H-NMDMRC(1)SASVEC(2)KQKC(3)YKAIGSIFGKC(1)MNKKC(2)KC(3)YPR-NH₂ |
| 51 | 52 | H-NMDMRC(1)SASVEC(2)KQKC(3)RKAIGSIFGKC(1)MNKKC(2)KC(3)YPR-NH₂ |
| 52 | 53 | H-NMDMRC(1)SASVEC(2)KQKC(3)LAAIGSIFGKC(1)MNKKC(2)KC(3)YPR-NH₂ |
| 53 | 54 | H-NMDMRC(1)SASVEC(2)KQKC(3)LYAIGSIFGKC(1)MNKKC(2)KC(3)YPR-NH₂ |
| 54 | 55 | H-NMDMRC(1)SASVEC(2)KQKC(3)LAIGSIFGKC(1)MNKKC(2)KC(3)YPR-NH₂ |
| 55 | 56 | H-NMDMRC(1)SASVEC(2)KQKC(3)LKYIGSIFGKC(1)MNKKC(2)KC(3)YPR-NH₂ |
| 56 | 57 | H-NMDMRC(1)SASVEC(2)KQKC(3)LKRIGSIFGKC(1)MNKKC(2)KC(3)YPR-NH₂ |
| 57 | 58 | H-NMDMRC(1)SASVEC(2)KQKC(3)LKIGSIFGKC(1)MNKKC(2)KC(3)YPR-NH₂ |
| 58 | 59 | H-NMDMRC(1)SASVEC(2)KQKC(3)LKAYGSIFGKC(1)MNKKC(2)KC(3)YPR-NH₂ |
| 59 | 60 | H-NMDMRC(1)SASVEC(2)KQKC(3)LKAEGSIFGKC(1)MNKKC(2)KC(3)YPR-NH₂ |
| 60 | 61 | H-NMDMRC(1)SASVEC(2)KQKC(3)LKARGSIFGKC(1)MNKKC(2)KC(3)YPR-NH₂ |
| 61 | 62 | H-NMDMRC(1)SASVEC(2)KQKC(3)LKAGSIFGKC(1)MNKKC(2)KC(3)YPR-NH₂ |
| 62 | 63 | H-N[Nle]D[Nle]RC(1)SASVEC(2)KQKC(3)LKAIGSPFGKC(1)[Nle]NKKC(2)KC(3)YPR- |
| | | NH₂ |
| 63 | 64 | H-N[Nle]D[Nle]RC(1)SASKEC(2)KQKC(3)LKAIGSIFGKC(1)[Nle]NKKC(2)KC(3)YPR- |
| | | NH₂ |
| 64 | 65 | H-H[Nle]D[Nle]RC(1)SASVEC(2)KQKC(3)LKAIGSIFGKC(1)[Nle]NKKC(2)KC(3)YPR- |
| | | NH₂ |
| 65 | 66 | H-Y[Nle]D[Nle]RC(1)SASVEC(2)KQKC(3)LKAIGSIFGKC(1)[Nle]NKKC(2)KC(3)YPR- |
| | | NH₂ |
| 66 | 67 | H-S[Nle]D[Nle]RC(1)SASVEC(2)KQKC(3)LKAIGSIFGKC(1)[Nle]NKKC(2)KC(3)YPR- |
| | | NH₂ |
| 66 | 68 | H-S[Nle]D[Nle]RC(1)SASVEC(2)KQKC(3)LKAIGSIFGKC(1)[Nle]NKKC(2)KC(3)YPR- |
| | | OH |
| 67 | 69 | H-V[Nle]D[Nle]RC(1)SASVEC(2)KQKC(3)LKAIGSIFGKC(1)[Nle]NKKC(2)KC(3)YPR- |
| | | NH₂ |
| 68 | 70 | H-S[Nle]D[Nle]RC(1)SA[Abu]VEC(2)KQKC(3)LKAIGSIFGKC(1)MNKKC(2)KC(3)YPR- |
| | | NH₂ |
| 69 | 71 | H-S[Nle]D[Nle]RC(1)SASVEC(2)KQKC(3)LKAIGSIFG[homo- |
| | | Lys]C(1)MNKKC(2)KC(3)YPR-NH₂ |
| 70 | 72 | H-S[Nle]D[Nle]RC(1)SASVEC(2)KQKC(3)LKAIGSIFGKC(1)MNKKC(2)KC(3)[F(4- |
| | | NH₂)]PR-NH₂ |
| 71 | 73 | H-S[Nle]D[Nle]RC(1)SASVEC(2)GQKC(3)LKAIGSIFGKC(1)MNKKC(2)KC(3)YPR- |
| | | NH₂ |
| 72 | 74 | H-S[Nle]D[Nle]RC(1)SASVEC(2)VQKC(3)LKAIGSIFGKC(1)MNKKC(2)KC(3)YPR-NH₂ |
| 73 | 75 | H-S[Nle]D[Nle]RC(1)SASVEC(2)K[2-Amino-5- |
| | | carboxypentanoyl]KC(3)LKAIGSIFGKC(1)MNKKC(2)KC(3)YPR-NH₂ |
| 74 | 76 | H-S[Nle]D[Nle]RC(1)SASVEC(2)KQKC(3)LKAIGSIFGKC(1)MNKKC(2)KC(3)YQ-NH₂ |
| 75 | 77 | H-S[Nle]D[Nle]RC(1)SASVEC(2)KQKC(3)LKAIGSIFGKC(1)MNKKC(2)RC(3)YPR- |
| | | NH₂ |
| 76 | 78 | H-S[Nle]DERC(1)SASVEC(2)KQKC(3)LKAIGSIFGKC(1)MNKKC(2)KC(3)YPR-NH₂ |
| 77 | 79 | H-S[Nle]D[Nle]RC(1)SASVEC(2)KQKC(3)LGAIGSIFGKC(1)MNKKC(2)KC(3)YPR- |
| | | NH₂ |
| 78 | 80 | H-S[Nle]D[Nle]RC(1)SASVEC(2)KQKC(3)LVAIGSIFGKC(1)MNKKC(2)KC(3)YPR-NH₂ |
| 79 | 81 | H-S[Nle]D[Nle]RC(1)SASVEC(2)AQSC(3)LKAIGSIFGKC(1)MNKKC(2)KC(3)YPR-NH₂ |
| 80 | 82 | H-S[Nle]D[Nle]RC(1)SASVEC(2)AQKC(3)LAAIGSIFGKC(1)MNKKC(2)KC(3)YPR-NH₂ |
| 81 | 83 | H-S[Nle]D[Nle]RC(1)SASVEC(2)AQLC(3)LAAIGSIFGKC(1)MNKKC(2)KC(3)YPR-NH₂ |
| 82 | 84 | H-P[Nle]E[Nle]RC(1)[2,4- |
| | | Diaminobutanoyl]ASVEC(2)KQKC(3)LAAIGSIFGKC(1)[Nle]NKKC(2)KC(3)YPR-OH |
| 83 | 85 | H- |
| | | P[Nle]D[Nle]RC(1)SASVEC(2)KQKC(3)LKAIGC(4)IFGKC(1)[Nle]NKKC(2)KC(3)YPC |
| | | (4)-NH₂ |
| 83 | 86 | H- |
| | | P[Nle]D[Nle]RC(1)SASVEC(2)KQKC(3)LKAIGC(4)IFGKC(1)[Nle]NKKC(2)KC(3)YPC |
| | | (4)-OH |
| 84 | 87 | H-S[Nle]D[Nle]RC(1)SASVEC(2)KEKC(3)LQAIGSIFGKC(1)[Nle]NKKC(2)KC(3)YPR- |
| | | NH₂ |
| 85 | 88 | H-P[Nle]D[Nle]RC(1)SASVEC(2)KEKC(3)L[homo- |
| | | Gln]AIGSIFGKC(1)[Nle]NKKC(2)KC(3)YPR-NH₂ |
| 86 | 89 | H-C(1)SASVEC(2)KQKC(3)LKAIGC(4)IFGKC(1)[Nle]NKKC(2)KC(3)YPC(4)-NH₂ |
| 87 | 90 | H- |
| | | S[Nle]D[Nle]RC(1)SASVEC(2)KQKC(3)LAAIGC(4)IFGKC(1)[Nle]NKKC(2)KC(3)YPC |
| | | (4)-NH₂ |
| 88 | 91 | H-P[Nle]E[Nle]RC(1)[2-Amino-3- |
| | | guanidinopropionyl]ASVEC(2)KQKC(3)LAAIGSIFGKC(1)[Nle]NKKC(2)KC(3)YPR-OH |
| 89 | 92 | H-S[Nle]D[Nle]RC(1)SALVEC(2)KQKC(3)LKAIGSIFGKC(1)MNKKC(2)KC(3)YPR-NH₂ |
| 90 | 93 | H-S[Nle]D[Nle]RC(1)SAVVEC(2)KQKC(3)LKAIGSIFGKC(1)MNKKC(2)KC(3)YPR-NH₂ |
| 91 | 94 | H-S[Nle]D[Nle]RC(1)SASVEC(2)KQKC(3)LKAIGSIFGKC(1)MNKKC(2)KC(3)[F(4- |
| | | F)]PR-NH₂ |
| 92 | 95 | H-S[Nle]D[Nle]RC(1)SASVEC(2)KQKC(3)LKAIGSIFGKC(1)MNKKC(2)KC(3)[F(4- |
| | | NO₂)]PR-NH₂ |
| 93 | 96 | H-S[Nle]D[Nle]RC(1)SASVEC(2)KQKC(3)LKAIGSIFGKC(1)MNKKC(2)KC(3)[F(4- |
| | | CH₃)]PR-NH₂ |
| 94 | 97 | H-[Nle]D[Nle]RC(1)SASVEC(2)KQKC(3)LKAIGSIFGKC(1)[Nle]NKKC(2)KC(3)YPR- |
| | | OH |
| 95 | 98 | H-NID[Nle]RC(1)SASVEC(2)KQKC(3)LKAIGSIFGKC(1)[Nle]NKKC(2)KC(3)YPR-OH |
| 96 | 99 | H-PIE[Nle]RC(1)SASVEC(2)KQKC(3)LKAIGSIFGKC(1)[Nle]NKKC(2)KC(3)YPR-OH |
| 97 | 100 | H-P[Nle]E[Nle]RC(1)SASVEC(2)KQKC(3)LAAIGSIFGKC(1)[Nle]NKKC(2)KC(3)YPR- |
| | | OH |
| 97 | 101 | H-P[Nle]E[Nle]RC(1)SASVEC(2)KQKC(3)LAAIGSIFGKC(1)[Nle]NKKC(2)KC(3)YPR- |
| | | NH₂ |
| 98 | 102 | H- |
| | | P[Nle]E[Nle]RC(1)SA[Orn]VEC(2)KQKC(3)LAAIGSIFGKC(1)[Nle]NKKC(2)KC(3)YPR- |
| | | OH |
| 99 | 103 | H-P[Nle]E[Nle]RC(1)SASVEC(2)KQKC(3)LLAIGSIFGKC(1)[Nle]NKKC(2)KC(3)YPR- |
| | | OH |
| 100 | 104 | H-PIDERC(1)SASVEC(2)KQKC(3)LAAIGSIFGKC(1)[Nle]NKKC(2)KC(3)YPR-OH |
| 101 | 105 | H-PIE[Nle]RC(1)SASVEC(2)KQKC(3)LAAIGSIFGKC(1)[Nle]NKKC(2)KC(3)YPR-OH |
| 102 | 106 | H-P[Nle]D[Nle]RC(1)SASVEC(2)AQKC(3)LAAIGSIFGKC(1)MNKKC(2)KC(3)YPR-OH |
| 103 | 107 | H-P[Nle]E[Nle]RC(1)SASVEC(2)AQKC(3)LAAIGSIFGKC(1)[Nle]NKKC(2)KC(3)YPR- |
| | | OH |
| 104 | 108 | H-C(1)SASVEC(2)KQKC(3)LKAIGSIFGKC(1)[Nle]NKKC(2)KC(3)YPR-NH₂ |
| 104 | 109 | Ac-C(1)SASVEC(2)KQKC(3)LKAIGSIFGKC(1)[Nle]NKKC(2)KC(3)YPR-NH₂ |
| 105 | 110 | H-P[Nle]E[Nle]RC(1)[2,3- |
| | | Diaminopropanoyl]ASVEC(2)KQKC(3)LAAIGSIFGKC(1)[Nle]NKKC(2)KC(3)YPR-OH |
| 106 | 111 | H-RC(1)SASVEC(2)KQKC(3)LKAIGSIFGKC(1)[Nle]NKKC(2)KC(3)YPR-NH₂ |
| 106 | 112 | Ac-RC(1)SASVEC(2)KQKC(3)LKAIGSIFGKC(1)[Nle]NKKC(2)KC(3)YPR-OH |
| 107 | 113 | Ac-SKC(1)SASVEC(2)KQKC(3)LKAIGSIFGKC(1)[Nle]NKKC(2)KC(3)YPR-NH₂ |
| 108 | 114 | Ac-LRC(1)SASVEC(2)KQKC(3)LKAIGSIFGKC(1)[Nle]NKKC(2)KC(3)YPR-NH₂ |
| 108 | 115 | H-LRC(1)SASVEC(2)KQKC(3)LKAIGSIFGKC(1)[Nle]NKKC(2)KC(3)YPR-OH |
| 109 | 116 | Ac-C(1)SASVEC(2)KQKC(3)LKAIGSIFGKC(1)[Nle]NKKC(2)KC(3)YPS-NH₂ |
| 110 | 117 | Ac-C(1)SASVEC(2)KQKC(3)LKAIGSIFGKC(1)[Nle]NKKC(2)KC(3)YS-NH₂ |
| 111 | 118 | Ac-C(1)SASVEC(2)KQKC(3)LKAIGSIFGKC(1)[Nle]NKKC(2)KC(3)YG-NH₂ |
| 112 | 119 | Ac-C(1)SASVEC(2)KQKC(3)LKAIGSIFGKC(1)[Nle]NKKC(2)KC(3)Y-NH₂ |
| 113 | 120 | H- |
| | | P[Nle]E[Nle]RC(1)[Orn]ASVEC(2)KQKC(3)LAAIGSIFGKC(1)[Nle]NKKC(2)KC(3)YPR- |
| | | OH |
| 114 | 12 | H-P[Nle]E[Nle]RC(1)HASVEC(2)KQKC(3)LAAIGSIFGKC(1)[Nle]NKKC(2)KC(3)YPR- |
| | | OH |
| 115 | 122 | Ac-LRC(1)SASVEC(2)KQKC(3)LAAIGSIFGKC(1)[Nle]NKKC(2)KC(3)YPR-OH |
| 116 | 123 | Ac-LRC(1)SASVEC(2)KQKC(3)LAAIGSIFGKC(1)MNKKC(2)KC(3)YPR-OH |
| 117 | 124 | H-C(1)SASVEC(2)KQKC(3)LKAIGSIFGKC(1)MNKKC(2)KC(3)YPR-OH |
| 118 | 125 | H-P[Nle]E[Nle]RC(1)SASVEC(2)KEKC(3)LAAIGSIFGKC(1)[Nle]NKKC(2)KC(3)YPR- |
| | | OH |
| 119 | 126 | H-p[Nle]E[Nle]RC(1)SASVEC(2)KQKC(3)LAAIGSIFGKC(1)[Nle]NKKC(2)KC(3)YPR- |
| | | OH |
| 120 | 127 | H-P[Nle]E[Nle]RC(1)SASVEC(2)KQKC(3)LAAIGSIFGKC(1)[Nle]NKKC(2)KC(3)YR- |
| | | OH |
| 121 | 128 | H-[Nle]E[Nle]RC(1)SASVEC(2)KQKC(3)LAAIGSIFGKC(1)[Nle]NKKC(2)KC(3)YPR- |
| | | OH |
| 122 | 129 | H- |
| | | P[Nle]E[Nle]RC(1)SASVEC(2)KQKC(3)LAAIGSIFGKC(1)[Nle]NKKC(2)KC(3)YPRR- |
| | | OH |
| 123 | 130 | H-P[Nle]E[Nle]RC(1)SASVEC(2)KQKC(3)LAAIGSIFGKC(1)[Nle]NKKC(2)KC(3)YPRY- |
| | | OH |
| 124 | 131 | H-P[Nle]E[Nle]RC(1)SASVEC(2)KQKC(3)LAAIGSIFGKC(1)[Nle]NKKC(2)KC(3)YPRL- |
| | | OH |
| 125 | 132 | H- |
| | | P[Nle]E[Nle]RC(1)SASVEC(2)KQKC(3)LAAIGSIFGKC(1)[Nle]NKKC(2)KC(3)YPRH- |
| | | OH |
| 126 | 133 | H-P[Nle]E[Nle]RC(1)SASVEC(2)KQKC(3)LAAIGSIFGKC(1)[Nle]NKKC(2)KC(3)YPRE- |
| | | OH |
| 127 | 134 | H- |
| | | P[Nle]E[Nle]RC(1)SASVEC(2)KQKC(3)LAAIGSIFGKC(1)[Nle]NKKC(2)KC(3)YPRKS- |
| | | OH |
| 128 | 135 | H- |
| | | P[Nle]E[Nle]RC(1)SASVEC(2)KQKC(3)LAAIGSIFGKC(1)[Nle]NKKC(2)KC(3)YPRFE- |
| | | OH |
| 129 | 136 | H- |
| | | P[Nle]E[Nle]RC(1)SASVEC(2)KQKC(3)LAAIGSIFGKC(1)[Nle]NKKC(2)KC(3)YPRHR- |
| | | OH |
| 130 | 137 | H- |
| | | P[Nle]E[Nle]RC(1)SASVEC(2)KQKC(3)LAAIGSIFGKC(1)[Nle]NKKC(2)KC(3)YPRAK- |
| | | OH |
| 131 | 138 | H-P[Nle]E[Nle]RC(1)SASVEC(2)KQKC(3)LAAIGSIFGKC(1)[Nle]NKKC(2)KC(3)YP- |
| | | [(4-amino-5-hydroxypentyl)guanidine] |
| 132 | 139 | H-P[Nle]E[Nle]RC(1)SASVEC(2)KQKC(3)LAAIGSIFGKC(1)[Nle]NKKC(2)KC(3)YPR- |
| | | [4-amino-5-hydroxypentanamide] |
| 133 | 140 | H- |
| | | P[Nle]E[Nle]RC(1)SASVEC(2)KQKC(3)LAAIGSIFGKC(1)[Nle]NKKC(2)KC(3)YPRST- |
| | | OH |
| 134 | 141 | H- |
| | | P[Nle]E[Nle]RC(1)SASVEC(2)KQKC(3)LAAIGSIFGKC(1)[Nle]NKKC(2)KC(3)YPRRY- |
| | | OH |
| 135 | 142 | H-P[Nle]E[Nle]RC(1)SSSVEC(2)KQKC(3)LAAIGSIFGKC(1)[Nle]NKKC(2)KC(3)YPR- |
| | | OH |
| 136 | 143 | H-P[Nle]E[Nle]RC(1)SLSVEC(2)KQKC(3)LAAIGSIFGKC(1)[Nle]NKKC(2)KC(3)YPR- |
| | | OH |
| 137 | 144 | H-P[Nle]E[Nle]RC(1)SAPVEC(2)KQKC(3)LAAIGSIFGKC(1)[Nle]NKKC(2)KC(3)YPR- |
| | | OH |
| 138 | 145 | H-P[Nle]E[Nle]RC(1)SASPEC(2)KQKC(3)LAAIGSIFGKC(1)[Nle]NKKC(2)KC(3)YPR- |
| | | OH |
| 139 | 146 | H-P[Nle]E[Nle]RC(1)SASQEC(2)KQKC(3)LAAIGSIFGKC(1)[Nle]NKKC(2)KC(3)YPR- |
| | | OH |
| 140 | 147 | H-P[Nle]E[Nle]RC(1)SASVEC(2)LQKC(3)LAAIGSIFGKC(1)[Nle]NKKC(2)KC(3)YPR- |
| | | OH |
| 141 | 148 | H-P[Nle]E[Nle]RC(1)SASVEC(2)KQPC(3)LAAIGSIFGKC(1)[Nle]NKKC(2)KC(3)YPR- |
| | | OH |
| 142 | 149 | H-P[Nle]E[Nle]RC(1)EASVEC(2)KQKC(3)LAAIGSIFGKC(1)[Nle]NKKC(2)KC(3)YPR- |
| | | OH |
| 143 | 150 | H-P[Nle]E[Nle]RC(1)FASVEC(2)KQKC(3)LAAIGSIFGKC(1)[Nle]NKKC(2)KC(3)YPR- |
| | | OH |
| 144 | 151 | H-P[Nle]E[Nle]RC(1)SYSVEC(2)KQKC(3)LAAIGSIFGKC(1)[Nle]NKKC(2)KC(3)YPR- |
| | | OH |
| 145 | 152 | H-P[Nle]E[Nle]RC(1)SAFVEC(2)KQKC(3)LAAIGSIFGKC(1)[Nle]NKKC(2)KC(3)YPR- |
| | | OH |
| 146 | 153 | H-P[Nle]E[Nle]RC(1)SA[2,3- |
| | | Diaminopropanoyl]VEC(2)KQKC(3)LAAIGSIFGKC(1)[Nle]NKKC(2)KC(3)YPR-OH |
| 147 | 154 | H-P[Nle]E[Nle]RC(1)[2,4- |
| | | Diaminobutanoyl]ASVEC(2)AQSC(3)LAAIGSIFGKC(1)[Nle]NGKC(2)KC(3)YPR-OH |
| 147 | 155 | H-P[Nle]E[Nle]RC(1)[2,4- |
| | | Diaminobutanoyl]ASVEC(2)AQSC(3)LAAIGSIFGKC(1)[Nle]NGKC(2)KC(3)YPR-NH2 |
| 148 | 156 | H-P[Nle]E[Nle]RC(1)RASVEC(2)AQSC(3)LAAIGSIFGKC(1)[Nle]NGKC(2)KC(3)YPR- |
| | | NH2 |
| 149 | 157 | H-P[Nle]E[Nle]RC(1)SASREC(2)AQSC(3)LAAIGSIFGKC(1)[Nle]NGKC(2)KC(3)YPR- |
| | | NH2 |
| 150 | 158 | H-P[Nle]E[Nle]RC(1)SASVEC(2)KQKC(3)LAKIGSIFGKC(1)[Nle]NKKC(2)KC(3)YPR- |
| | | OH |

In some embodiments, the Kv1.3 inhibitor or pharmaceutical salt of the invention comprises a peptide consisting of any one of peptides 1 to 158. In some embodiments, the Kv1.3 inhibitor or pharmaceutical salt of the invention consists of any one of peptides 1 to 158. In preferred embodiments, the Kv1.3 inhibitor or pharmaceutical salt of the invention comprises peptide 100. In preferred embodiments, the Kv1.3 inhibitor or pharmaceutical salt of the invention consists of peptide 100.

### Synthesis of Kv1.3 Inhibitors of the Invention

The Kv1.3 inhibitors described herein may be synthesised by means of solid-phase or liquid-phase peptide synthesis methodology. In this context, reference may be made to WO 98/11125 and, among many others, Fields, G. B. et al., 2002, “Principles and practice of solid-phase peptide synthesis”. In: Synthetic Peptides (2nd Edition), and the Examples herein. Alternatively, the Kv1.3 inhibitors described herein may be synthesised by recombinant techniques, or by a combination of recombinant techniques and peptide chemistry.

An exemplary method for producing a Kv1.3 inhibitor of the invention comprises synthesising the peptide by means of solid-phase or liquid-phase peptide synthesis methodology and recovering the peptide; or expressing the peptide from a nucleic acid construct that encodes the peptide and recovering the expression product; or expressing a precursor peptide from a nucleic acid construct that encodes the precursor peptide sequence, recovering the expression product, and modifying the precursor peptide to yield the Kv1.3 inhibitor.

The precursor peptide may be modified by introduction of one or more non-proteinogenic amino acids (e.g. Nle), introduction of the appropriate terminal groups R¹ and R², etc.

Expression of the peptide or precursor peptide from a nucleic acid encoding the peptide or precursor peptide may be performed in a cell or a cell-free expression system comprising such a nucleic acid. Such expression typically requires that the peptide or precursor peptide is composed entirely of proteinogenic amino acids (i.e. the 20 amino acids encoded by the standard genetic code.)

For recombinant expression, the nucleic acid fragments encoding the precursor peptide will normally be inserted in suitable vectors to form cloning or expression vectors. The vectors can, depending on purpose and type of application, be in the form of plasmids, phages, cosmids, mini-chromosomes, or virus, but also naked DNA which is only expressed transiently in certain cells is an important vector. Preferred cloning and expression vectors (plasmid vectors) are capable of autonomous replication, thereby enabling high copy-numbers for the purposes of high-level expression or high-level replication for subsequent cloning.

In general outline, an expression vector comprises the following features in the 5′->3′ direction and in operable linkage: a promoter for driving expression of the nucleic acid fragment, optionally a nucleic acid sequence encoding a leader peptide enabling secretion (to the extracellular phase or, where applicable, into the periplasm), the nucleic acid fragment encoding the precursor peptide, and optionally a nucleic acid sequence encoding a terminator. They may comprise additional features such as selectable markers and origins of replication. When operating with expression vectors in producer strains or cell lines it may be preferred that the vector is capable of integrating into the host cell genome. The skilled person is very familiar with suitable vectors and is able to design one according to their specific requirements.

Such vectors are used to transform host cells to produce the peptide or precursor peptide. Such transformed cells can be cultured cells or cell lines used for propagation of the nucleic acid fragments and vectors, and/or used for recombinant production of the precursor peptides.

Preferred transformed cells are micro-organisms such as bacteria [such as the species Escherichia (e.g. E. coli), Bacillus (e.g. Bacillus subtilis), Salmonella, or Mycobacterium (preferably non-pathogenic, e.g. M. bovis BCG), yeasts (e.g., Saccharomyces cerevisiae and Pichia pastoris), and protozoans. Alternatively, the transformed cells may be derived from a multicellular organism, i.e. it may be fungal cell, an insect cell, an algal cell, a plant cell, or an animal cell such as a mammalian cell. For the purposes of cloning and/or optimised expression it is preferred that the transformed cell is capable of replicating the nucleic acid fragment of the invention. Cells expressing the nucleic fragment can be used for small-scale or large-scale preparation of the peptides of the invention. When producing the peptide or precursor peptide by means of transformed cells, it is convenient, although far from essential, that the expression product is secreted into the culture medium.

### Treating or Preventing a Disease or Disorder

The invention provides a Kv1.3 inhibitor, or a pharmaceutically acceptable salt thereof, as described herein for use in a method of treating or preventing a disease or disorder in a subject, wherein the method comprises administering the Kv1.3 inhibitor to the subject once every 2 to 8 days.

Accordingly, the invention also provides a method of treating or preventing a disease or disorder in a subject, the method comprising administering the Kv1.3 inhibitor or pharmaceutically acceptable salt of the invention to the subject once every 2 to 8 days.

The invention also provides use of a Kv1.3 inhibitor, or a pharmaceutically acceptable salt thereof, as described herein in manufacture of a medicament for use in a method of treating or preventing a disease or disorder in a subject, wherein the method comprises administering the Kv1.3 inhibitor to the subject once every 2 to 8 days.

In other words, the invention provides medical uses of the Kv1.3 inhibitor or pharmaceutically acceptable salt described herein. The invention provides a therapeutic, preventative or prophylactic method comprising administering to a subject the Kv1.3 inhibitor or pharmaceutically acceptable salt described herein. In all cases, the medical use described may also be phrased as a method of treating or preventing a disease or disorder, the method comprising administering the Kv1.3 inhibitor or pharmaceutically acceptable salt of the invention to the subject.

The term “subject” is used interchangeably herein with “patient” and “individual”, and refers to either a human or a non-human animal. These terms include mammals such as humans, primates, livestock animals (e.g. bovines and porcines), companion animals (e.g. canines and felines) and rodents (e.g. mice and rats). The subject as described herein suffers from the disease or disorder as described herein.

The terms “disease” and “disorder” refer to a state of dysfunction of the body. The term disease is herein synonymous with similar terms such as “condition” or “disorder”. Thus, the terms “disease”, “condition” and “disorder” are interchangeable herein. In some embodiments, the disease or disorder is a disease or disorder that may be treated or prevented (i.e. is treatable or preventable) using the Kv1.3 inhibitor as described herein.

The terms “treating”, “treatment” and “treats” refer to alleviating, reducing or eliminating the symptoms of a disease in a subject. The term “treating” thus encompasses curing of the disease or disorder, but does not require that the disease or disorder is completely eliminated from the subject. Even a mild reduction of the symptoms of a disease or disorder is “treating” that disease or disorder. The terms “treating”, “treatment” and “treats” refer to both treatment of an existing disease in the subject and prevention of a disease in the subject, i.e. prophylaxis. It will therefore be recognized that treatment as referred to herein may in some embodiments be prophylactic.

The term “preventing”, “prevention” or “prevents” relates to a prophylactic treatment, i.e. to a measure or procedure the purpose of which is to prevent a disease or disorder arising, rather than to treat an existing disease or disorder. Prevention means that a desired pharmacological and/or physiological effect is obtained that is prophylactic in terms of completely or partially preventing a disease or disorder or symptom thereof.

### Treating Inflammation

As described herein, inhibitors of Kv1.3 may be useful in reducing inflammation. The data presented in the Examples herein directly demonstrate that treating a subject with a Kv1.3 inhibitor reduces inflammation.

Accordingly, in some embodiments, the disease or disorder is an inflammatory disease or disorder. An inflammatory disease or disorder is any disease, condition or disorder in which reduction of inflammation is desirable, e.g. where inflammation contributes to symptoms or pathogenesis.

In some embodiments the inflammatory condition or disorder is selected from an autoimmune disorder, an allergy or hypersensitivity, allograft rejection, transplant rejection, graft-versus-host disease, hay fever, asthma, anaphylaxis, allergic rhinitis, urticaria, eczema, alopecia areata, dermatomyositis, inclusion body myositis, polymyositis, ankylosing spondylitis, vasculitis, arthritis (including rheumatoid arthritis, osteoarthritis, psoriatic arthritis), Sjogren's syndrome, systemic lupus erythematosus (SLE), uveitis, inflammatory fibrosis (e.g. scleroderma, lung fibrosis, cirrhosis), chronic obstructive pulmonary disease (COPD), hepatitis, chronic inflammatory demyelinating polyneuropathy, inflammatory bowel disease, colitis (e.g. Crohn's disease and ulcerative colitis), erythema, thyroiditis, psoriasis, atopic dermatitis, allergic contact dermatitis, scleroderma, glomerulonephritis, inflammatory bone resorption, multiple sclerosis and type 1 diabetes. In preferred embodiments, the disease or disorder is arthritis, such as rheumatoid arthritis, osteoarthritis or psoriatic arthritis.

In some embodiments, the invention provides a Kv1.3 inhibitor or pharmaceutically acceptable salt of the invention as described herein for use in a method of inhibiting or reducing inflammation.

In some embodiments, the subject has reduced inflammation following administration of the Kv1.3 inhibitor. Inflammation may be measured using techniques known in the art (e.g. by measuring cytokine levels in the subject). In some embodiments, following administration of the Kv1.3 inhibitor inflammation in the subject is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, following administration of the Kv1.3 inhibitor inflammation in the subject is reduced by 100% (i.e. inflammation is eliminated).

### Treating Metabolic Diseases

Inhibitors of Kv1.3 may also have beneficial metabolic effects, e.g. in relation to energy homeostasis, body weight regulation, and glucose control.

In some embodiments, the disease or disorder is a metabolic disease or disorder. A metabolic disease or disorder is any disease, condition or disorder caused by or associated with abnormal metabolism (i.e. any disruption of the processing of food by the body to maintain life). A metabolic disease or disorder may be characterised by the presence in the body of a subject of too much or too little of particular chemicals, such as proteins, carbohydrates, lipids, their constituent molecules (e.g. amino acids, sugars, fatty acids) and other such biological molecules.

Obesity may be considered a metabolic disorder, given that an obese subject has excess fat and may have abnormal metabolic processes. Thus, in some embodiments the disease or disorder is obesity, obesity linked inflammation, obesity linked gallbladder disease or obesity induced sleep apnoea.

In some embodiments, the disease or disorder is a disease or disorder caused by or associated with impaired glucose control. Such diseases including metabolic syndrome, insulin resistance, glucose intolerance, pre-diabetes, increased fasting glucose and type 2 diabetes. Some of these conditions can be associated with obesity. Their effects on these conditions may be mediated in whole or in part via an effect on body weight, or may be independent thereof.

In some embodiments the invention provides a Kv1.3 inhibitor or pharmaceutically acceptable salt of the invention as described herein for use in a method of inhibiting weight gain, promoting weight loss, reducing excess body weight or treating obesity (e.g. by control of appetite, feeding, food intake, calorie intake, and/or energy expenditure). An effect on body weight may be therapeutic or cosmetic.

### Treating Proliferating Cells and Cancer

Kv1.3 is also expressed in proliferating human and mouse smooth muscle cells. Inhibitors of Kv1.3 may be effective in smooth muscle proliferative disorders such as restenosis, e.g. in patients following vascular surgery (e.g. angioplasty). Thus, in some embodiments, the disease or disorder is a smooth muscle proliferative disorder. In some embodiments the smooth muscle proliferative disorder is restenosis.

Further evidence suggests that Kv1.3 channels are involved in the activation and/or proliferation of many types of cells, including tumor cells (Bielanska et al., Curr. Cancer Drug Targets 9:904-14, 2009), microglia (Khanna et al., Am. J. Physiol. Cell Physiol. 280: C796-806, 2001) and differentiation of neuronal progenitor cells (Wang et al., J. Neurosci. 30:5020-7, 2010). Kv1.3 inhibitors may therefore be beneficial in the treatment of neuroinflammatory and neurodegenerative disorders. Thus, in some embodiments, the disease or disorder is a neuroinflammatory or neurodegenerative disease or disorder. In some embodiments the neuroinflammatory or neurodegenerative disease or disorder is Alzheimer's disease, multiple sclerosis (MS), Parkinson's disease or amyotrophic lateral sclerosis (ALS) (e.g. following viral infections).

In some embodiments the disease or disorder is cancer. In some embodiments the cancer is breast cancer, prostate cancer or lymphoma. In some embodiments the lymphoma is non-Hodgkin lymphoma (NHL). Non-Hodgkin lymphomas include T-cell NHL and B-cell NHL. Forms of B-cell NHL include diffuse large B-cell lymphoma, follicular lymphoma, Burkitt lymphoma, immunoblastic large cell lymphoma, precursor B-lymphoblastic lymphoma and mantle cell lymphoma. Forms of T-cell NHL include mycosis fungoides, anaplastic large cell lymphoma, peripheral T-cell lymphoma, precursor T-lymphoblastic lymphoma and Sézary syndrome.

### Administration of Kv1.3 Inhibitor

The invention provides a Kv1.3 inhibitor, or a pharmaceutically acceptable salt thereof, as described herein for use in a method of treating or preventing a disease or disorder in a subject, wherein the method comprises administering the Kv1.3 inhibitor to the subject once every 2 to 8 days. The method thus comprises administration of the Kv1.3 inhibitor to the subject once every 2 to 8 days. In otherwords, the Kv1.3 inhibitor is administered to the subject once every 2 to 8 days.

The terms “administering” and “administration” refer to providing the Kv1.3 inhibitor into the body of the subject. “An administration” refers to a single event of administering the Kv1.3 inhibitor to the subject. Administration of the Kv1.3 inhibitor may be by any mode of administration common or standard in the art, such as oral, intravenous, intramuscular, subcutaneous, sublingual, intranasal or intradermal administration, by a suppository route or by implanting. In some embodiments, the Kv1.3 inhibitor is administered by injection, preferably subcutaneous injection.

### Timing of Administration

According to the invention, the Kv1.3 inhibitor is administered to the subject once every 2 to 8 days.

In other words, according to the invention, the Kv1.3 inhibitor is administered in a particular dosage to the subject once every 2 to 8 days. According to some aspects of the invention, the Kv1.3 inhibitor is administered to the subject once every 3 to 8 days. In other words, according to some aspects of the invention, the Kv1.3 inhibitor is administered in a particular dosage to the subject once every 3 to 8 days. According to some aspects of the invention, the Kv1.3 inhibitor is administered to the subject once every 3 to 7 days. In other words, according to some aspects of the invention, the Kv1.3 inhibitor is administered in a particular dosage to the subject once every 3 to 7 days.

Kv1.3 inhibitors based on the PaT1 toxin peptide have a short half-life in the body of a subject. For instance, Example 6 herein shows that such Kv1.3 inhibitors have a half-life of about 1 hour in a rat model organism. However, it has been surprisingly found that, despite this short half-life, the Kv1.3 inhibitors described herein have a long-lasting effect on T cells. In particular, Example 8 herein shows that inflammation is reduced in a rat ear inflammation model up to 7 days after treatment with the Kv1.3 inhibitor. Additionally, Example 9 herein shows that treatment with a Kv1.3 inhibitor once every 5 days reduces inflammation in a rat model of arthritis. Without wishing to be bound by theory, it is hypothesised that the Kv1.3 inhibitor somehow, through its interaction with the Kv1.3 ion channel, “reprograms” T cells to remain inactive for an extended period, thereby suppressing inflammation for multiple days.

These surprising findings support the concept of treating a subject suffering from a disease, condition or disorder that may be treated using a Kv1.3 inhibitor as described herein following a regime of administering the inhibitor at roughly weekly intervals (i.e. once every 2 to 8 days). It is advantageous to increase as much as possible the period between administrations of an active compound as this saves time and effort (as fewer administrations need to be carried out over a given period) and expense (as less active compound needs to be used over a given period). Particularly in the case of active compounds administered by certain routes, such as subcutaneous injection, a longer interval between administrations also increases patient comfort and may thereby increase patient compliance with the treatment regime. For example, administering the Kv1.3 inhibitor in accordance with the present invention (i.e. once every 2 to 8 days) is superior to administering the inhibitor daily, for the above reasons. An unexpected finding described in the present disclosure is that administering a Kv1.3 inhibitor as described herein once every 2 to 8 days is possible despite their short in vivo half-life, as an effect of the inhibitor (such as reduced inflammation) is observed multiple days after administration.

Thus, the method of treating a disease or disorder comprises administering the Kv1.3 inhibitor to the subject once every 2 to 8 days. In other words, the Kv1.3 inhibitor is administered to the subject at least 2 days after the previous administration of the Kv1.3 inhibitor to the subject (if any) and at most 8 days after the previous administration of the Kv1.3 inhibitor to the subject. The period between administrations of the Kv1.3 inhibitor to the subject may be termed the “interval” between administrations of the Kv1.3 inhibitor. Thus, the method comprises administering the Kv1.3 inhibitor to the subject at intervals of 2 to 8 days (i.e. the interval between administrations is 2 to 8 days in duration). The method comprises administering the Kv1.3 inhibitor to the subject at an interval of 2 to 8 days.

The term “day” refers to a period of 24 hours±8 hours (i.e. 16 to 32 hours). In other words, “1 day” is approximately 24 hours, with an allowed error of 8 hours before and after the exact 24-hour time-point. The error of 8 hours is not cumulative over multiple days. Accordingly, “2 days” refers to a period of 48 hours±8 hours (i.e. 40 to 56 hours) rather than a period of 48 hours±16 hours, “3 days” refers to a period of 72 hours±8 hours (i.e. 64 to 80 hours), “4 days” refers to a period of 96 hours±8 hours (i.e. 88 to 104 hours), “5 days” refers to a period of 120 hours±8 hours (i.e. 112 to 128 hours), “6 days” refers to a period of 144 hours±8 hours (i.e. 136 to 152 hours), “7 days” refers to a period of 168 hours±8 hours (i.e. 160 to 176 hours) and “8 days” refers to a period of 192 hours±8 hours (i.e. 184 to 200 hours).

Thus, the expression “administering the Kv1.3 inhibitor to the subject once every 2 to 8 days” (i.e. once every 2 days to 8 days) herein means administering the Kv1.3 inhibitor to the subject once every 40 to 200 hours. Thus, the Kv1.3 inhibitor is administered to the subject 40 hours to 200 hours after the previous administration (if any). In other words, the Kv1.3 inhibitor is administered to the subject at least 40 hours after the previous administration of the Kv1.3 inhibitor to the subject (if any) and at most 200 hours after the previous administration of the Kv1.3 inhibitor to the subject (if any).

In some embodiments, the method comprises administering the Kv1.3 inhibitor to the subject once every 2 to 7 days, such as once every 2 to 6 days, once every 2 to 5 days, once every 2 to 4 days or once every 2 to 3 days. In some embodiments, the method comprises administering the Kv1.3 inhibitor to the subject once every 3 to 8 days, such as once every 3 to 7 days, once every 3 to 6 days, once every 3 to 5 days or once every 3 to 4 days. In some embodiments, the method comprises administering the Kv1.3 inhibitor to the subject once every 4 to 8 days, such as once every 4 to 7 days, once every 4 to 6 days or once every 4 to 5 days. In some embodiments, the method comprises administering the Kv1.3 inhibitor to the subject once every 5 to 8 days, such as once every 5 to 7 days or once every 5 to 6 days. In some embodiments, the method comprises administering the Kv1.3 inhibitor to the subject once every 6 to 8 days, such as once every 6 to 7 days. In some embodiments, the method comprises administering the Kv1.3 inhibitor to the subject once every 7 to 8 days.

In some embodiments, the method comprises administering the Kv1.3 inhibitor to the subject once every 2 days, once every 3 days, once every 4 days, once every 5 days, once every 6 days, once every 7 days or once every 8 days. Preferably, the method comprises administering the Kv1.3 inhibitor to the subject once every 7 days. Preferably, the method comprises administering the Kv1.3 inhibitor to the subject approximately once weekly. Preferably, the method comprises administering the Kv1.3 inhibitor to the subject once weekly.

The method comprises administering the Kv1.3 inhibitor to the subject once every 2 to 8 days within an administration period. An “administration period” is an overall period of time during which the subject is being administered the Kv1.3 inhibitor (i.e. a period of time during which the Kv1.3 is being administered at intervals to the subject). In other words, the administration period is a period beginning with a first administration of the Kv1.3 inhibitor during that administration period and ending with a final administration of the inhibitor during that administration period. The duration of an administration period may depend upon various factors, including whether the disease or disorder is being treated or prevented, the type of disease or disorder being treated or prevented, and characteristics of the subject (e.g. age, weight or immunological status). An administration period may be determined by a clinician prescribing the Kv1.3 inhibitor to the subject.

The administration period may be as long as necessary to treat or prevent the disease or disorder in the subject (i.e. administration of the Kv1.3 inhibitor may continue for as long as is necessary). In some embodiments, the administration period is at least 1 month. In other words, in some embodiments, the method comprises administering the Kv1.3 inhibitor to the subject once every 2 to 8 days for at least 1 month. In some embodiments, the administration period is at least 2 months, such as at least 3 months, at least 4 months, at least 5 months, at least 6 months, at least 7 months, at least 8 months, at least 9 months, at least 10 months, at least 11 months or at least 12 months (i.e. at least 1 year). In some embodiments, the administration period is at least 1 year. In other words, in some embodiments, the method comprises administering the Kv1.3 inhibitor to the subject once every 2 to 8 days for at least 1 year. In some embodiments, the administration period is at least 2 years, such as at least 3 years, at least 4 years, at least 5 years, at least 6 years, at least 7 years, at least 8 years, at least 9 years, at least 10 years, at least 11 years, at least 12 years, at least 13 years, at least 14 years, at least 15 years, at least 16 years, at least 17 years, at least 18 years, at least 19 years or at least 20 years. In some embodiments, the administration period is the lifetime of the subject. In other words, in some embodiments, the method comprises administering the Kv1.3 inhibitor to the subject once every 2 to 8 days for the lifetime of the subject.

A given subject may experience multiple (i.e. more than one) administration periods. In other words, the Kv1.3 inhibitor may be administered to the subject over a given administration period, then administration of the Kv1.3 inhibitor ceases, then the Kv1.3 inhibitor is administered to the subject again for a further administration period. There may be multiple such cessations and resumptions of administration of the Kv1.3 inhibitor over the lifetime of the subject (i.e. multiple administration periods, such as 2, 3, 4, 5, 6, 7, 8, 9, 10 or more administration periods). An administration period of the invention may be any one (or more) of these multiple administration periods, and this is not precluded by one or more other administration periods (of these multiple administration periods) not being within the scope of the invention.

The number of administrations of the Kv1.3 inhibitor to the subject within an administration period (i.e. the number of times the Kv1.3 inhibitor is administered to the subject within an administration period) depends on the duration of the administration period and the time that elapses between administrations (i.e. the interval between administrations). For example, the longer the administration period, the more total administrations of the Kv1.3 inhibitor to the subject there are likely to be within that administration period. The Kv1.3 inhibitor may be administered any number of times to the subject (i.e. the method comprises administering the Kv1.3 inhibitor to the subject any number of times). In some embodiments, the method comprises administering the Kv1.3 inhibitor to the subject at least 2 times (i.e. 2 or more times) within the administration period. In other words, in some embodiments, the method comprises at least 2 administrations of the Kv1.3 inhibitor to the subject within the administration period. In some embodiments, the method comprises administering the Kv1.3 inhibitor to the subject at least 3 times (i.e. 3 or more times) within the administration period, such as at least 4 times, at least 5 times, at least 6 times, at least 7 times, at least 8 times, at least 9 times, at least 10 times, at least 15 times, at least 20 times, at least 30 times, at least 40 times, at least 50 times, at least 60 times, at least 70 times, at least 80 times, at least 90 times or at least 100 times within the administration period.

Typically, the interval between administrations of the Kv1.3 inhibitor (i.e. the amount of time that elapses from a given administration until the next administration) is the same throughout the administration period. In other words, in some embodiments the interval between administrations of the Kv1.3 inhibitor is constant or uniform throughout the administration period. For example, the Kv1.3 inhibitor may be administered once every 7 days (i.e. the interval between all administrations is 7 days). A constant interval is preferred as this may improve patient compliance with treatment as it is simpler to administer the inhibitor at constant intervals (e.g. it may be easier for the subject/patient to remember to administer the inhibitor). However, in some embodiments, the interval varies. In other words, in some embodiments, each interval is independently selected to be a duration of from 2 days to 8 days. For example, the Kv1.3 inhibitor may be administered 2 days after the previous administration, which may have been 8 days after the administration prior to that, which may have been 5 days after the administration prior to that etc. The interval may be varied in this manner upon instruction of a clinician seeking to achieve a particular clinical outcome. For example, the interval may be increased over the course of the administration period to determine the maximum possible length of the interval whilst maintaining treatment or prevention of the disease or disorder.

According to the invention, the method comprises administering the Kv1.3 inhibitor to the subject once every 2 to 8 days. However, within a given administration period, there may be intervals of more than 8 days duration. It will be understood that the invention encompasses administration of the Kv1.3 inhibitor once every 2 to 8 days for any portion of an administration period. For example, administering the Kv1.3 inhibitor to the subject once every 2 to 8 days for an initial portion of the administration period, followed by administering the Kv1.3 inhibitor to the subject at intervals longer than 8 days is within the scope of the invention. Thus, according to the invention, the method of treating or preventing the disease or disorder comprises administering the Kv1.3 inhibitor to the subject at least twice, wherein the interval between at least two administrations of the Kv1.3 inhibitor is 2 to 8 days. In some embodiments, the method comprises administering the Kv1.3 inhibitor to the subject, wherein there is an interval of 2 to 8 days between at least two administrations of the Kv1.3 inhibitor. In some embodiments, the method comprises at least one interval (i.e. interval between administrations of the Kv1.3 inhibitor) of 2 to 8 days. In other words, in some embodiments, the method comprises a first administration of the Kv1.3 inhibitor to the subject followed by a second administration of the Kv1.3 inhibitor 2 to 8 days later. The first and second administrations may be preceded by or followed by further administrations of the Kv1.3 inhibitor to the subject. In some embodiments, the method consists of a first administration of the Kv1.3 inhibitor to the subject followed by a second administration of the Kv1.3 inhibitor 2 to 8 days later (i.e. there are no further administrations of the Kv1.3 inhibitor).

### Dosage of Kv1.3 Inhibitor

The term “dosage” refers to the amount of Kv1.3 inhibitor administered to the subject in a single administration. For example, in embodiments wherein the Kv1.3 inhibitor is administered via subcutaneous injection, “dosage” may refer to the amount of Kv1.3 inhibitor in a single injection of the inhibitor. The terms “dosage” and “dose” are used interchangeably herein.

In some embodiments, the method comprises administering the Kv1.3 inhibitor in ascending dosages.

In some embodiments, the method comprises administering the Kv1.3 inhibitor to the subject at a dosage of 0.1 mg to 30.0 mg.

In other words, in some embodiments, the Kv1.3 inhibitor is administered to the subject at a dosage of 0.1 mg to 30.0 mg. In other words, in some embodiments, the Kv1.3 inhibitor is administered to the subject at a dosage of not less than 0.1 mg and not more than 30.0 mg. In some embodiments, the Kv1.3 inhibitor is formulated at a dosage of 0.1 mg to 30.0 mg. In some embodiments, the method comprises administering the Kv1.3 inhibitor to the subject once every 2 to 8 days at a dosage of 0.1 mg to 30.0 mg.

In some embodiments, each administration of the Kv1.3 inhibitor to the subject is at the same dosage as the other administrations. In some embodiments, each administration of the Kv1.3 inhibitor to the subject may be at a different dosage to the other administrations. In other words, each administration of the Kv1.3 inhibitor to the subject may be independently selected to be at a dosage of 0.1 mg to 30.0 mg.

In some embodiments, the Kv1.3 inhibitor is administered to the subject in a single dosage formulation of 0.1 mg to 30.0 mg. This single dosage formulation may be administered to the subject once or multiple times wherein each of the multiple dosage formulations for administration to the subject need not comprise the same amount of the Kv1.3 inhibitor. In other words, the Kv1.3 inhibitor may be administered to the subject in a series of single administrations wherein each of the single administrations may not comprise the same amount of the Kv1.3 inhibitor.

In some embodiments, the method comprises administering the Kv1.3 inhibitor to the subject at a dosage of 1.0 mg to 30.0 mg, such as 2.0 mg to 30.0 mg, 3.0 mg to 30.0 mg, 4.0 mg to 30.0 mg, 5.0 mg to 30.0 mg, 6.0 mg to 30.0 mg, 7.0 mg to 30.0 mg, 8.0 mg to 30.0 mg, 9.0 mg to 30.0 mg, 10.0 mg to 30.0 mg, 11.0 mg to 30.0 mg, 12.0 mg to 30.0 mg, 13.0 mg to 30.0 mg, 14.0 mg to 30.0 mg, 15.0 mg to 30.0 mg, 16.0 mg to 30.0 mg, 17.0 mg to 30.0 mg, 18.0 mg to 30.0 mg, 19.0 mg to 30.0 mg, 20.0 mg to 30.0 mg, 21.0 mg to 30.0 mg, 22.0 mg to 30.0 mg, 23.0 mg to 30.0 mg, 24.0 mg to 30.0 mg, 25.0 mg to 30.0 mg, 26.0 mg to 30.0 mg, 27.0 mg to 30.0 mg, 28.0 mg to 30.0 mg or 29.0 mg to 30.0 mg.

In some embodiments, the method comprises administering the Kv1.3 inhibitor to the subject at a dosage of 1.0 mg to 29.0 mg, such as 1.0 mg to 28.0 mg, 1.0 mg to 27.0 mg, 1.0 mg to 26.0 mg, 1.0 mg to 25.0 mg, 1.0 mg to 24.0 mg, 1.0 mg to 23.0 mg, 1.0 mg to 22.0 mg, 1.0 mg to 21.0 mg, 1.0 mg to 20.0 mg, 1.0 mg to 19.0 mg, 1.0 mg to 18.0 mg, 1.0 mg to 17.0 mg, 1.0 mg to 16.0 mg, 1.0 mg to 15.0 mg, 1.0 mg to 14.0 mg, 1.0 mg to 13.0 mg, 1.0 mg to 12.0 mg, 1.0 mg to 11.0 mg, 1.0 mg to 10.0 mg, 1.0 mg to 9.0 mg, 1.0 mg to 8.0 mg, 1.0 mg to 7.0 mg, 1.0 mg to 6.0 mg, 1.0 mg to 5.0 mg, 1.0 mg to 4.0 mg, 1.0 mg to 3.0 mg or 1.0 mg to 2.0 mg.

In some embodiments, the method comprises administering the Kv1.3 inhibitor to the subject at a dosage of 0.1 mg to 15.0 mg, such as 0.1 mg to 10.0 mg, 0.1 mg to 9.0 mg, 0.1 mg to 8.0 mg, 0.1 mg to 7.0 mg, 0.1 mg to 6.0 mg, 0.1 mg to 5.0 mg, 0.1 mg to 4.0 mg, 0.1 mg to 3.0 mg, 0.1 mg to 2.0 mg, 0.1 mg to 1.0 mg or 0.1 mg to 0.5 mg.

In some embodiments, the method comprises administering the Kv1.3 inhibitor to the subject at a dosage of about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 11.0 mg, about 12.0 mg, about 13.0 mg, about 14.0 mg, about 15.0 mg, about 16.0 mg, about 17.0 mg, about 18.0 mg, about 19.0 mg, about 20.0 mg, about 21.0 mg, about 22.0 mg, about 23.0 mg, about 24.0 mg, about 25.0 mg about, about 26.0 mg, about 27.0 mg, about 28.0 mg, about 29.0 mg or about 30.0 mg.

In some embodiments, the method comprises administering the Kv1.3 inhibitor to the subject at a dosage of 10 nmol/kg to 400 nmol/kg.

In other words, in some embodiments, the Kv1.3 inhibitor is administered to the subject at a dosage of 10 nmol/kg to 400 nmol/kg. In other words, in some embodiments, the Kv1.3 inhibitor is administered to the subject at a dosage of not less than 10 nmol/kg and not more than 400 nmol·kg. In some embodiments, the Kv1.3 inhibitor is formulated at a dosage of 10 nmol/kg to 400 nmol/kg. In some embodiments, the method comprises administering the Kv1.3 inhibitor to the subject once every 2 to 8 days at a dosage of 10 nmol/kg to 400 nmo/kg. In some embodiments, each administration of the Kv1.3 inhibitor to the subject may be independently selected to be at a dose of 10 nmol/kg to 400 nmol/kg.

The units of “nmol/kg” mean that the dosage of the Kv1.3 inhibitor is relative to the weight/body mass of the subject. A given number of nanomoles of Kv1.3 is administered per kilogram of body mass of the subject. The precise dosage of the Kv1.3 inhibitor may therefore be determined by a clinician on a subject-by-subject basis.

In some embodiments, the method comprises administering the Kv1.3 inhibitor to the subject at a dosage of 50 nmol/kg to 400 nmol/kg, such as 100 nmol/kg mg to 400 nmol/kg, 150 nmol/kg mg to 400 nmol/kg, 200 nmol/kg mg to 400 nmol/kg, 250 nmol/kg mg to 400 nmol/kg, 300 nmol/kg mg to 400 nmol/kg or 350 nmol/kg mg to 400 nmol/kg.

In some embodiments, the method comprises administering the Kv1.3 inhibitor to the subject at a dosage of 10 nmol/kg to 350 nmol/kg, such as 10 nmol/kg mg to 350 nmol/kg, 10 nmol/kg mg to 300 nmol/kg, 10 nmol/kg mg to 250 nmol/kg, 10 nmol/kg mg to 200 nmol/kg, 10 nmol/kg mg to 150 nmol/kg, 10 nmol/kg mg to 100 nmol/kg or 10 nmol/kg mg to 50 nmol/kg.

In some embodiments, the method comprises administering the Kv1.3 inhibitor to the subject at a dosage of about 3 nmol/kg, about 10 nmol/kg, about 50 nmol/kg, about 100 nmol/kg, about 150 nmol/kg, about 200 nmol/kg, about 250 nmol/kg, about 300 nmol/kg, about 350 nmol/kg or about 400 nmol/kg. Preferably, the method comprises administering the Kv1.3 inhibitor to the subject at a dosage of about 300 nmol/kg.

In some embodiments, the method comprises administering the Kv1.3 inhibitor to the subject at a dosage of about 3 nmol/kg to about 300 nmol/kg.

In other words, in some embodiments, the Kv1.3 inhibitor is administered to the subject at a dosage of 3 nmol/kg to 300 nmol/kg. In some embodiments, the Kv1.3 inhibitor is administered to the subject at a dosage of not less than 3 nmol/kg and not more than 300 nmol/kg. In some embodiments, the Kv1.3 inhibitor is formulated at a dosage of 3 nmol/kg to 300 nmol/kg. In some embodiments, the method comprises administering the Kv1.3 inhibitor to the subject once every 2 to 8 days at a dosage of 3 nmol/kg to 300 nmo/kg. In some embodiments, each administration of the Kv1.3 inhibitor to the subject may be independently selected to be at a dose of 3 nmol/kg to 300 nmol/kg.

In some embodiments, the method comprises administering the Kv1.3 inhibitor to the subject at a dosage of 50 nmol/kg to 300 nmol/kg, such as 100 nmol/kg mg to 300 nmol/kg, 150 nmol/kg mg to 300 nmol/kg, 200 nmol/kg mg to 300 nmol/kg, or 250 nmol/kg mg to 300 nmol/kg.

In some embodiments, the method comprises administering the Kv1.3 inhibitor to the subject at a dosage of 3 nmol/kg to 250 nmol/kg, such as 3 nmol/kg mg to 200 nmol/kg, 3 nmol/kg mg to 150 nmol/kg, 3 nmol/kg mg to 100 nmol/kg or 3 nmol/kg mg to 50 nmol/kg.

### Pharmaceutical Composition

The invention also provides a pharmaceutical composition comprising a Kv1.3 inhibitor or pharmaceutically acceptable salt as described herein for use in a method as described herein. Thus, the invention provides a pharmaceutical composition comprising a Kv1.3 inhibitor or pharmaceutically acceptable salt as described herein for use in a method of treating or preventing a disease or disorder in a subject, wherein the method comprises administering the pharmaceutical composition to the subject once every 2 to 8 days.

In other words, in some embodiments, the invention provides a Kv1.3 inhibitor or pharmaceutically acceptable salt thereof for use as described herein, wherein the Kv1.3 inhibitor or pharmaceutically acceptable salt is in the form of a composition. Preferably the composition is a pharmaceutical composition. In some embodiments, the pharmaceutical composition comprises a pharmaceutically acceptable carrier, excipient or vehicle. In some embodiments, a pharmaceutical composition of the invention is one wherein the Kv1.3 inhibitor is in the form of a pharmaceutically acceptable acid addition salt.

In some embodiments, the composition comprises one or more Kv1.3 inhibitor(s) as described herein (i.e. more than one Kv1.3 inhibitor). Each of the Kv1.3 inhibitors is independently selected from any of the Kv1.3 inhibitors as described herein. In other words, each of the Kv1.3 inhibitors in the composition may be any one of the Kv1.3 inhibitors as described herein. In some embodiments, the composition comprises one or more peptides. In some embodiments, the composition comprises one or more peptides wherein each of the peptides comprises or consists of a sequence independently selected from any of the peptide sequences described herein. In some embodiments, the composition comprises one or more peptides wherein each of the peptides comprises or consists of a peptide independently selected from any of the peptides described herein.

In some embodiments, the pharmaceutical composition comprises a therapeutically effective amount of the Kv1.3 inhibitor or pharmaceutically acceptable salt thereof. As will be apparent to one skilled in the medical art, a “therapeutically effective amount” of a compound or pharmaceutical composition of the present invention will vary depending upon, inter alia, the age, weight and/or gender of the subject (patient) to be treated. Other factors that may be of relevance include the physical characteristics of the specific patient under consideration, the patient's diet, the nature of any concurrent medication, the particular compound(s) employed, the particular mode of administration, the desired pharmacological effect(s) and the particular therapeutic indication. Because these factors and their relationship in determining this amount are well known in the medical arts, the determination of therapeutically effective dosage levels to achieve the desired therapeutic effect will be within the ambit of the skilled person.

As used herein, the term “a therapeutically effective amount” refers to an amount which reduces symptoms of a given disease, disorder, condition or pathology, and preferably which normalizes physiological responses in an individual with that disease, disorder, condition or pathology. Reduction of symptoms or normalization of physiological responses can be determined using methods routine in the art and may vary with a given disease, disorder, condition or pathology. In one aspect, a therapeutically effective amount of a Kv1.3 inhibitor of the invention, or a pharmaceutical composition, is an amount which restores a measurable physiological parameter to substantially the same value of the parameter in an individual without the disease, disorder, condition or pathology in question.

In some embodiments of the invention, administration of a Kv1.3 inhibitor or pharmaceutical composition of the present invention is commenced at lower dosage levels, with dosage levels being increased until the desired effect of preventing/treating the relevant medical indication is achieved. This would define a therapeutically effective amount. For the compounds of the present invention, alone or as part of a pharmaceutical composition, such human doses of the active compound may be between about 0.01 pmol/kg and 500 μmol/kg body weight, between about 0.01 pmol/kg and about 300 μmol/kg body weight, between about 0.01 pmol/kg and about 100 μmol/kg body weight, between about 0.1 pmol/kg and about 50 μmol/kg body weight, between about 1 pmol/kg and about 10 μmol/kg body weight, between about 5 pmol/kg and about 5 μmol/kg body weight, between about 10 pmol/kg and about 1 μmol/kg body weight, between about 50 pmol/kg and about 0.1 μmol/kg body weight, between about 100 pmol/kg and about 0.01 μmol/kg body weight, between about 0.001 pmol/kg and about 0.5 μmol/kg body weight, between about 0.05 pmol/kg and about 0.1 μmol/kg body weight.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

### Patent Terminology

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

All patents, published patent applications and non-patent publications referred to in this application are specifically incorporated by reference herein. In case of conflict, the present specification, including its specific definitions, will control.

Each embodiment of the invention described herein may be taken alone or in combination with one or more other embodiments of the invention.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5 to 3′ orientation; amino acid sequences are written left to right in amino to carboxy orientation, respectively.

As used herein, the singular forms “a”, “an”, and “the” include plural referents unless the context clearly dictates otherwise.

Throughout this specification, the word “comprise”, and grammatical variants thereof, such as “comprises” or “comprising”, will be understood to imply the inclusion of a stated integer or component, or group of integers or components, but not the exclusion of any other integer or component, or group of integers or components. The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”. The term “including” is used to mean “including but not limited to”. “Including” and “including but not limited to” may be used interchangeably.

The publications discussed herein are provided solely for their disclosure prior to the filing date of the present application. Nothing herein is to be construed as an admission that such publications constitute prior art to the aspects appended hereto.

The invention will now be further described by way of Examples, which are meant to serve to assist one of ordinary skill in the art in carrying out the invention and are not intended in any way to limit the scope of the invention.

### SEQUENCE LISTING

Sequence identifier numbers (SEQ ID NOs) are assigned to sequences herein as follows:
SEQ ID NOs: 1-150—Kv1.3 inhibitor peptide sequences
SEQ ID NOs: 151-180 & 182-188—Segments of Kv1.3 inhibitor peptide sequences
SEQ ID NO: 181—Skipped

### EXAMPLES

### Example 1: General Peptide Synthesis

Lists of abbreviations and suppliers are provided in Table 1 below.

TABLE 1

| | Abbreviation | Name | Brand / Supplier |
|---|---|---|---|
| Resins | None | TentaGel ™ PHB AA(Proct)-Fmoc | Rapp Polymere |
| | None | TentaGel ™ SRAM | Rapp Polymere |
| Amino | None | Pseudoprolines (E.g. QT, AT, FS) | Jupiter Bioscience Ltd. |
| acids | None | Fmoc-L-AA-OH | Senn Chemicals AG |
| | None | Fmoc-L-Lys(N3)-OH | Iris Biotech |
| Building | None | 10-Undecynoic acid | Chem-Impex |
| blocks | None | Alkynyl Myristic acid | Chem-Impex |
| | None | Alkynyl Palmitic acid | Chem-Impex |
| | None | Alkynyl Stearic acid | Chem-Impex |
| Coupling | COMU | (1-Cyano-2-ethoxy-2- | Watson International Ltd. |
| reagents | | oxoethylidenaminooxy)dimethyl- | |
| | | amino-morpholino-carbenium | |
| | | hexafluorophosphate | |
| | DIC | Diisopropylcarbodiimide | Fluka / Sigma Aldrich Co. |
| | HATU | N-[(dimethylamino)-1H-1,2,3- | ChemPep Inc. |
| | | triazol[4,5-b]pyridine-1- | |
| | | ylmethylene]-N- | |
| | | methylmethanaminium | |
| | | hexafluorophosphate N-oxide | |
| | HOBt | Hydroxybenzotriazole | Sigma-Aldrich Co. |
| Solvents | Boc₂O | Di-tert-butyl pyrocarbonate | Advanced ChemTech |
| reagents | DCM | Dichloromethane | Prolabo (VWR) |
| | DIPEA | Diisopropylethylamine | Fluka / Sigma Aldrich Co. |
| | DMF | N,N-dimethylformamide | Taminco |
| | DODT | 3,6-dioxa-1,8-octanedithiol | Sigma-Aldrich Co. |
| | Et₂O | Diethyl ether | Prolabo (VWR) |
| | EtOH | Ethanol | CCS Healthcare AB |
| | None | Formic acid (HPLC) | Sigma-Aldrich Co. |
| | H₂O | Water, Milli-Q water | Millipore |
| | MeCN | Acetonitrile (HPLC) | Sigma-Aldrich Co. |
| | MeOH | Methanol | Sigma-Aldrich Co. |
| | NMP | N-methylpyrrolidone | Sigma-Aldrich Co. |
| | None | Piperidine | Jubliant Life Sciences Ltd. |
| | TFA | Trifluoroacetic acid (HPLC) | Chemicals Raw Materials Ltd. |
| | TIS | Triisopropylsilane | Sigma-Aldrich Co. |

### Apparatus and Synthetic Strategy

Peptides were synthesized batchwise on a peptide synthesiser, such as a CEM Liberty Peptide Synthesizer or a Symphony X Synthesizer, according to solid phase peptide synthetic procedures using 9-fluorenylmethyloxycarbonyl (Fmoc) as N-α-amino protecting group and suitable common protection groups for side-chain functionalities.

As polymeric support based resins, such as e.g. TentaGel™, was used. The synthesizer was loaded with resin that prior to usage was swelled in DMF.

### Coupling

### CEM Liberty Peptide Synthesizer

A solution of Fmoc-protected amino acid (4 equiv.) was added to the resin together with a coupling reagent solution (4 equiv.) and a solution of base (8 equiv.). The mixture was either heated by the microwave unit to 70-75° C. and coupled for 5 minutes or coupled with no heat for 60 minutes. During the coupling nitrogen was bubbled through the mixture.

### Symphony X Synthesizer

The coupling solutions were transferred to the reaction vessels in the following order: amino acid (4 equiv.), HATU (4 equiv.) and DIPEA (8 equiv.). The coupling time was 10 min at room temperature (RT) unless otherwise stated. The resin was washed with DMF (5×0.5 min). In case of repeated couplings the coupling time was in all cases 45 min at RT.

### Deprotection

### CEM Liberty Peptide Synthesizer

The Fmoc group was deprotected using piperidine in DMF or other suitable solvents. The deprotection solution was added to the reaction vessel and the mixture was heated for 30 sec. reaching approx. 40° C. The reaction vessel was drained and fresh deprotection solution was added and subsequently heated to 70-75° C. for 3 min. After draining the reaction vessel the resin was washed with DMF or other suitable solvents.

### Symphony X Synthesizer

Fmoc deprotection was performed for 2.5 minutes using 40% piperidine in DMF and repeated using the same conditions. The resin was washed with DMF (5×0.5 min).

### Cleavage

The dried peptide resin was treated with TFA and suitable scavengers for approximately 2 hours. The volume of the filtrate was reduced and the crude peptide was precipitated after addition of diethylether. The crude peptide precipitate was washed several times with diethylether and finally dried.

### HPLC Purification of the Crude Peptide

The crude peptide was purified by preparative reverse phase HPLC using a conventional HPLC apparatus, such as a Gilson GX-281 with 331/332 pump combination′, for binary gradient application equipped with a column, such as 5×25 cm Gemini NX 5u C18 110 A column, and a fraction collector using a flow 20-40 ml/min with a suitable gradient of buffer A (0.1% Formic acid, aq.) or A (0.1% TFA, aq.) and buffer B (0.1% Formic acid, 90% MeCN, aq.) or B (0.1% TFA, 90% MeCN, aq.). Fractions were analyzed by analytical HPLC and MS and selected fractions were pooled and lyophilized. The final product was characterized by HPLC and MS.

### Disulphide Formation

The crude or partially purified linear peptide with six cysteines was dissolved in a buffer such as sodium hydrogen carbonate (NaHCO₃) or ammonium acetate (NH₄Ac) to give a final concentration of approximate 0.1 mg/ml or 25 μM. The pH of the buffer was adjusted to pH 8.0 and the solution was stirred at room temperature under magnetic stirring and open access to the atmosphere. The progress of the reaction was determined by HPLC and was usually evaluated to be complete overnight. The solution was quenched by reducing the pH of the solution by an organic acid such as acetic acid or trifluoroacetic acid (pH<4). The solution was filtered and loaded directly on a prep-HPLC column for purification.

### Analytical HPLC

Final purities were determined by analytic HPLC (Agilent 1100/1200 series) equipped with auto sampler, degasser, 20 μl flow cell and Chromeleon software. The HPLC was operated with a flow of 1.2 ml/min at 40° C. using an analytical column, such as Kinetex 2.6 μm XB-C18 100 A 100×4.6 mm column. The compound was detected and quantified at 215 nm. Buffers A (0.1% TFA, aq.) and buffer B (0.1% TFA, 90% MeCN, aq.).

### Mass Spectroscopy

Final MS analysis was performed on a conventional mass spectrometer, e.g. Waters Xevo G2 Tof, equipped with electrospray detector with lock-mass calibration and MassLynx software. It was operated in positive mode using direct injection and a cone voltage of 15V (1 TOF), 30 V (2 TOF) or 45 V (3 TOF) as specified on the chromatogram. Precision was 5 ppm with a typical resolution of 15,000-20,000.

Peptides (i.e. Kv1.3 inhibitors) synthesised are shown in Table 2:

TABLE 2

| SEQ | | Cpd No. in | |
|---|---|---|---|
| ID | Ptd | PCT/EP20 | |
| NO | No. | 20/076187 | Peptide |
| 1 | 1 | 1 | H- |
| | | | QMDMRC(1)SASVEC(2)KQKC(3)LKAIGSIFGKC(1)MNKKC(2)KC(3)YPR- |
| | | | OH |
| 2 | 2 | 2 | H- |
| | | | QMDMRC(1)SASVEC(2)KQKC(3)LKAIGRGFGKC(1)MNKKC(2)KC(3)YPR- |
| | | | OH |
| 3 | 3 | 3 | H- |
| | | | NMDMRC(1)SASVEC(2)KQKC(3)LKAIGSIFGKC(1)MNKKC(2)KC(3)YPR- |
| | | | OH |
| 5 | 6 | 5 | H-MDMRC(1)SASVEC(2)KQKC(3)LKAIGSIFGKC(1)MNKKC(2)KC(3)YPR- |
| | | | OH |
| 8 | 9 | 8 | H-NIDMRC(1)SASVEC(2)KQKC(3)LKAIGSIFGKC(1)MNKKC(2)KC(3)YPR- |
| | | | OH |
| 12 | 13 | 12 | H- |
| | | | NMEMRC(1)SASVEC(2)KQKC(3)LKAIGSIFGKC(1)MNKKC(2)KC(3)YPR- |
| | | | OH |
| 16 | 17 | 16 | H-NMDMRC(1)SASVEC(2)KQKC(3)LKAIGSIFGKC(1) |
| | | | [Nle]NKKC(2)KC(3)YPR-NH₂ |
| 17 | 18 | 17 | H- |
| | | | NMDMRC(1)SASVEC(2)KVKC(3)LKAIGSIFGKC(1)MNKKC(2)KC(3)YPR- |
| | | | NH₂ |
| 18 | 19 | 18 | H- |
| | | | NMDMRC(1)SASVEC(2)KQLC(3)LKAIGSIFGKC(1)MNKKC(2)KC(3)YPR- |
| | | | NH₂ |
| 19 | 20 | 19 | H- |
| | | | NMDMRC(1)SASVEC(2)KQKC(3)KKAIGSIFGKC(1)MNKKC(2)KC(3)YPR- |
| | | | NH₂ |
| 20 | 21 | 20 | H- |
| | | | NMDMRC(1)SASVEC(2)KQKC(3)LDAIGSIFGKC(1)MNKKC(2)KC(3)YPR- |
| | | | NH₂ |
| 21 | 22 | 21 | H- |
| | | | NMDMRC(1)SASVEC(2)KQKC(3)LKAIRSIFGKC(1)MNKKC(2)KC(3)YPR- |
| | | | NH₂ |
| 22 | 23 | 22 | H- |
| | | | NMDMRC(1)SASVEC(2)KQKC(3)LKAIESIFGKC(1)MNKKC(2)KC(3)YPR- |
| | | | NH₂ |
| 23 | 24 | 23 | H- |
| | | | NMDMRC(1)SASVEC(2)KQKC(3)LKAIGSIFGKC(1)MNKKC(2)KC(3)YPRR |
| | | | RTA-NH₂ |
| 25 | 26 | 25 | H- |
| | | | NMDMRC(1)KASVEC(2)KQKC(3)LKAIGSIFGKC(1)MNKKC(2)KC(3)YPR- |
| | | | NH₂ |
| 26 | 27 | 26 | H- |
| | | | NMDMRC(1)SISVEC(2)KQKC(3)LKAIGSIFGKC(1)MNKKC(2)KC(3)YPR- |
| | | | NH₂ |
| 27 | 28 | 27 | H- |
| | | | NMDMRC(1)SASREC(2)KQKC(3)LKAIGSIFGKC(1)MNKKC(2)KC(3)YPR- |
| | | | NH₂ |
| 28 | 29 | 28 | H- |
| | | | NMDMRC(1)SASVQC(2)KQKC(3)LKAIGSIFGKC(1)MNKKC(2)KC(3)YPR- |
| | | | NH₂ |
| 29 | 30 | 29 | H- |
| | | | NMDMRC(1)SASVEC(2)LQKC(3)LKAIGSIFGKC(1)MNKKC(2)KC(3)YPR- |
| | | | NH₂ |
| 30 | 31 | 30 | H- |
| | | | NMDMRC(1)SASVEC(2)AQKC(3)LKAIGSIFGKC(1)MNKKC(2)KC(3)YPR- |
| | | | NH₂ |
| 31 | 32 | 31 | H- |
| | | | NMDMRC(1)SASVEC(2)KEKC(3)LKAIGSIFGKC(1)MNKKC(2)KC(3)YPR- |
| | | | NH₂ |
| 32 | 33 | 32 | H- |
| | | | NMDMRC(1)SASVEC(2)KLKC(3)LKAIGSIFGKC(1)MNKKC(2)KC(3)YPR- |
| | | | NH₂ |
| 33 | 34 | 33 | H- |
| | | | NMDMRC(1)SASVEC(2)KQKC(3)LKAIHSIFGKC(1)MNKKC(2)KC(3)YPR- |
| | | | NH₂ |
| 34 | 35 | 34 | H- |
| | | | NMDMRC(1)SASVEC(2)KQKC(3)LKAIGSKFGKC(1)MNKKC(2)KC(3)YPR- |
| | | | NH₂ |
| 35 | 36 | 35 | H- |
| | | | NMDMRC(1)SASVEC(2)KQKC(3)LKAIGSRFGKC(1)MNKKC(2)KC(3)YPR- |
| | | | NH₂ |
| 36 | 37 | 36 | H- |
| | | | NMDMRC(1)SASVEC(2)KQKC(3)LKAIGSIFGKC(1)MNGKC(2)KC(3)YPR- |
| | | | NH₂ |
| 37 | 38 | 37 | H- |
| | | | NMDMRC(1)SASVEC(2)KQKC(3)LKAIGSIFGKC(1)MNKKC(2)HC(3)YPR- |
| | | | NH₂ |
| 38 | 39 | 38 | H- |
| | | | NMDMRC(1)SASVEC(2)KQKC(3)LKAIGSIFGKC(1)MNKKC(2)VC(3)YPR- |
| | | | NH₂ |
| 39 | 40 | 39 | H- |
| | | | N[Nle]D[Nle]RC(1)SASVEC(2)KQKC(3)LKAIGSIFGKC(1)MNKKC(2)KC(3) |
| | | | YPR-NH₂ |
| 40 | 41 | 40 | H- |
| | | | N[Nle]D[Nle]RC(1)SASVEC(2)KQKC(3)LKAIGSIFGKC(1)[Nle]NKKC(2)KC |
| | | | (3)YPR-NH₂ |
| 41 | 42 | 41 | H- |
| | | | P[Nle]D[Nle]RC(1)SASVEC(2)KQKC(3)LKAIGSIFGKC(1)[Nle]NKKC(2)KC |
| | | | (3)YPR-NH₂ |
| 42 | 43 | 42 | H- |
| | | | N[Nle]D[Nle]RC(1)RASVEC(2)KQKC(3)LKAIGSIFGKC(1)[Nle]NKKC(2)KC |
| | | | (3)YPR-NH₂ |
| 43 | 44 | 43 | H- |
| | | | N[Nle]D[Nle]RC(1)SASVEC(2)EQKC(3)LKAIGSIFGKC(1)[Nle]NKKC(2)KC |
| | | | (3)YPR-NH₂ |
| 44 | 45 | 44 | H- |
| | | | N[Nle]D[Nle]RC(1)SASVEC(2)QQKC(3)LKAIGSIFGKC(1)[Nle]NKKC(2)KC |
| | | | (3)YPR-NH₂ |
| 45 | 46 | 45 | H- |
| | | | N[Nle]D[Nle]RC(1)SASVEC(2)KKKC(3)LKAIGSIFGKC(1)[Nle]NKKC(2)KC |
| | | | (3)YPR-NH₂ |
| 46 | 47 | 46 | H- |
| | | | N[Nle]S[Nle]RC(1)SASVEC(2)KQKC(3)LKAIGSIFGKC(1)[Nle]NKKC(2)KC |
| | | | (3)YPR-NH₂ |
| 47 | 48 | 47 | H- |
| | | | N[Nle]D[Nle]RC(1)SHSVEC(2)KQKC(3)LKAIGSIFGKC(1)[Nle]NKKC(2)KC |
| | | | (3)YPR-NH₂ |
| 48 | 49 | 48 | H- |
| | | | N[Nle]D[Nle]RC(1)SASVEC(2)KQSC(3)LKAIGSIFGKC(1)[Nle]NKKC(2)KC |
| | | | (3)YPR-NH₂ |
| 49 | 50 | 49 | H- |
| | | | N[Nle]D[Nle]RC(1)SASVEC(2)KQKC(3)KAIGSIFGKC(1)[Nle]NKKC(2)KC |
| | | | (3)YPR-NH₂ |
| 50 | 51 | 50 | H- |
| | | | NMDMRC(1)SASVEC(2)KQKC(3)YKAIGSIFGKC(1)MNKKC(2)KC(3)YPR- |
| | | | NH₂ |
| 51 | 52 | 51 | H- |
| | | | NMDMRC(1)SASVEC(2)KQKC(3)RKAIGSIFGKC(1)MNKKC(2)KC(3)YPR- |
| | | | NH₂ |
| 52 | 53 | 52 | H- |
| | | | NMDMRC(1)SASVEC(2)KQKC(3)LAAIGSIFGKC(1)MNKKC(2)KC(3)YPR- |
| | | | NH₂ |
| 53 | 54 | 53 | H- |
| | | | NMDMRC(1)SASVEC(2)KQKC(3)LYAIGSIFGKC(1)MNKKC(2)KC(3)YPR- |
| | | | NH₂ |
| 54 | 55 | 54 | H-NMDMRC(1)SASVEC(2)KQKC(3)LAIGSIFGKC(1)MNKKC(2)KC(3)YPR- |
| | | | NH₂ |
| 55 | 56 | 55 | H- |
| | | | NMDMRC(1)SASVEC(2)KQKC(3)LKYIGSIFGKC(1)MNKKC(2)KC(3)YPR- |
| | | | NH₂ |
| 56 | 57 | 56 | H- |
| | | | NMDMRC(1)SASVEC(2)KQKC(3)LKRIGSIFGKC(1)MNKKC(2)KC(3)YPR- |
| | | | NH₂ |
| 58 | 59 | 58 | H- |
| | | | NMDMRC(1)SASVEC(2)KQKC(3)LKAYGSIFGKC(1)MNKKC(2)KC(3)YPR- |
| | | | NH₂ |
| 62 | 63 | 62 | H- |
| | | | N[Nle]D[Nle]RC(1)SASVEC(2)KQKC(3)LKAIGSPFGKC(1)[Nle]NKKC(2)KC |
| | | | (3)YPR-NH₂ |
| 63 | 64 | 63 | H- |
| | | | N[Nle]D[Nle]RC(1)SASKEC(2)KQKC(3)LKAIGSIFGKC(1)[Nle]NKKC(2)KC |
| | | | (3)YPR-NH₂ |
| 64 | 65 | 64 | H- |
| | | | H[Nle]D[Nle]RC(1)SASVEC(2)KQKC(3)LKAIGSIFGKC(1)[Nle]NKKC(2)KC |
| | | | (3)YPR-NH₂ |
| 65 | 66 | 65 | H- |
| | | | Y[Nle]D[Nle]RC(1)SASVEC(2)KQKC(3)LKAIGSIFGKC(1)[Nle]NKKC(2)KC |
| | | | (3)YPR-NH₂ |
| 66 | 67 | 66 | H- |
| | | | S[Nle]D[Nle]RC(1)SASVEC(2)KQKC(3)LKAIGSIFGKC(1)[Nle]NKKC(2)KC |
| | | | (3)YPR-NH₂ |
| 66 | 68 | 82 | H- |
| | | | S[Nle]D[Nle]RC(1)SASVEC(2)KQKC(3)LKAIGSIFGKC(1)[Nle]NKKC(2)KC |
| | | | (3)YPR-OH |
| 67 | 69 | 67 | H- |
| | | | V[Nle]D[Nle]RC(1)SASVEC(2)KQKC(3)LKAIGSIFGKC(1)[Nle]NKKC(2)KC |
| | | | (3)YPR-NH₂ |
| 68 | 70 | 68 | H- |
| | | | S[Nle]D[Nle]RC(1)SA[Abu]VEC(2)KQKC(3)LKAIGSIFGKC(1)MNKKC(2)KC |
| | | | (3)YPR-NH₂ |
| 69 | 71 | 69 | H-S[Nle]D[Nle]RC(1)SASVEC(2)KQKC(3)LKAIGSIFG[homo- |
| | | | Lys]C(1)MNKKC(2)KC(3)YPR-NH₂ |
| 70 | 72 | 70 | H- |
| | | | S[Nle]D[Nle]RC(1)SASVEC(2)KQKC(3)LKAIGSIFGKC(1)MNKKC(2)KC(3) |
| | | | [F(4-NH₂)]PR-NH₂ |
| 71 | 73 | 71 | H- |
| | | | S[Nle]D[Nle]RC(1)SASVEC(2)GQKC(3)LKAIGSIFGKC(1)MNKKC(2)KC(3) |
| | | | YPR-NH₂ |
| 72 | 74 | 72 | H- |
| | | | S[Nle]D[Nle]RC(1)SASVEC(2)VQKC(3)LKAIGSIFGKC(1)MNKKC(2)KC(3) |
| | | | YPR-NH₂ |
| 73 | 75 | 73 | H-S[Nle]D[Nle]RC(1)SASVEC(2)K[2-Amino-5- |
| | | | carboxypentanoyl]KC(3)LKAIGSIFGKC(1)MNKKC(2)KC(3)YPR-NH₂ |
| 74 | 76 | 74 | H- |
| | | | S[Nle]D[Nle]RC(1)SASVEC(2)KQKC(3)LKAIGSIFGKC(1)MNKKC(2)KC(3) |
| | | | YQ-NH₂ |
| 75 | 77 | 75 | H- |
| | | | S[Nle]D[Nle]RC(1)SASVEC(2)KQKC(3)LKAIGSIFGKC(1)MNKKC(2)RC(3) |
| | | | YPR-NH₂ |
| 76 | 78 | 76 | H- |
| | | | S[Nle]DERC(1)SASVEC(2)KQKC(3)LKAIGSIFGKC(1)MNKKC(2)KC(3)YP |
| | | | R-NH₂ |
| 78 | 80 | 78 | H- |
| | | | S[Nle]D[Nle]RC(1)SASVEC(2)KQKC(3)LVAIGSIFGKC(1)MNKKC(2)KC(3) |
| | | | YPR-NH₂ |
| 79 | 81 | 79 | H- |
| | | | S[Nle]D[Nle]RC(1)SASVEC(2)AQSC(3)LKAIGSIFGKC(1)MNKKC(2)KC(3) |
| | | | YPR-NH₂ |
| 80 | 82 | 80 | H- |
| | | | S[Nle]D[Nle]RC(1)SASVEC(2)AQKC(3)LAAIGSIFGKC(1)MNKKC(2)KC(3) |
| | | | YPR-NH₂ |
| 82 | 84 | None | H-P[Nle]E[Nle]RC(1)[2,4- |
| | | | Diaminobutanoyl]ASVEC(2)KQKC(3)LAAIGSIFGKC(1)[Nle]NKKC(2)KC(3) |
| | | | YPR-OH |
| 83 | 85 | 83 | H- |
| | | | P[Nle]D[Nle]RC(1)SASVEC(2)KQKC(3)LKAIGC(4)IFGKC(1)[Nle]NKKC(2)K |
| | | | C(3)YPC(4)-NH₂ |
| 83 | 86 | 88 | H- |
| | | | P[Nle]D[Nle]RC(1)SASVEC(2)KQKC(3)LKAIGC(4)IFGKC(1)[Nle]NKKC(2)K |
| | | | C(3)YPC(4)-OH |
| 84 | 87 | 84 | H- |
| | | | S[Nle]D[Nle]RC(1)SASVEC(2)KEKC(3)LQAIGSIFGKC(1)[Nle]NKKC(2)KC |
| | | | (3)YPR-NH₂ |
| 85 | 88 | 85 | H-P[Nle]D[Nle]RC(1)SASVEC(2)KEKC(3)L[homo- |
| | | | Gln]AIGSIFGKC(1)[Nle]NKKC(2)KC(3)YPR-NH₂ |
| 86 | 89 | 86 | H- |
| | | | C(1)SASVEC(2)KQKC(3)LKAIGC(4)IFGKC(1)[Nle]NKKC(2)KC(3)YPC(4)- |
| | | | NH₂ |
| 87 | 90 | 87 | H- |
| | | | S[Nle]D[Nle]RC(1)SASVEC(2)KQKC(3)LAAIGC(4)IFGKC(1)[Nle]NKKC(2)K |
| | | | C(3)YPC(4)-NH₂ |
| 88 | 91 | None | H-P[Nle]E[Nle]RC(1)[2-Amino-3- |
| | | | guanidinopropionyl]ASVEC(2)KQKC(3)LAAIGSIFGKC(1)[Nle]NKKC(2)KC |
| | | | (3)YPR-OH |
| 91 | 94 | 91 | H- |
| | | | S[Nle]D[Nle]RC(1)SASVEC(2)KQKC(3)LKAIGSIFGKC(1)MNKKC(2)KC(3) |
| | | | [F(4-F)]PR-NH₂ |
| 92 | 95 | 92 | H- |
| | | | S[Nle]D[Nle]RC(1)SASVEC(2)KQKC(3)LKAIGSIFGKC(1)MNKKC(2)KC(3) |
| | | | [F(4-NO₂)]PR-NH₂ |
| 93 | 96 | 93 | H- |
| | | | S[Nle]D[Nle]RC(1)SASVEC(2)KQKC(3)LKAIGSIFGKC(1)MNKKC(2)KC(3) |
| | | | [F(4-CH₃)]PR-NH₂ |
| 94 | 97 | 94 | H-[Nle]D[Nle]RC(1)SASVEC(2)KQKC(3)LKAIGSIFGKC(1) |
| | | | [Nle]NKKC(2)KC(3)YPR-OH |
| 95 | 98 | 95 | H- |
| | | | NID[Nle]RC(1)SASVEC(2)KQKC(3)LKAIGSIFGKC(1)[Nle]NKKC(2)KC(3)Y |
| | | | PR-OH |
| 96 | 99 | 96 | H- |
| | | | PIE[Nle]RC(1)SASVEC(2)KQKC(3)LKAIGSIFGKC(1)[Nle]NKKC(2)KC(3)Y |
| | | | PR-OH |
| 97 | 100 | 97 | H- |
| | | | P[Nle]E[Nle]RC(1)SASVEC(2)KQKC(3)LAAIGSIFGKC(1)[Nle]NKKC(2)KC |
| | | | (3)YPR-OH |
| 97 | 101 | 98 | H- |
| | | | P[Nle]E[Nle]RC(1)SASVEC(2)KQKC(3)LAAIGSIFGKC(1)[Nle]NKKC(2)KC |
| | | | (3)YPR-NH₂ |
| 99 | 103 | 99 | H- |
| | | | P[Nle]E[Nle]RC(1)SASVEC(2)KQKC(3)LLAIGSIFGKC(1)[Nle]NKKC(2)KC |
| | | | (3)YPR-OH |
| 100 | 104 | 100 | H- |
| | | | PIDERC(1)SASVEC(2)KQKC(3)LAAIGSIFGKC(1)[Nle]NKKC(2)KC(3)YPR- |
| | | | OH |
| 101 | 105 | 101 | H- |
| | | | PIE[Nle]RC(1)SASVEC(2)KQKC(3)LAAIGSIFGKC(1)[Nle]NKKC(2)KC(3)Y |
| | | | PR-OH |
| 102 | 106 | 102 | H- |
| | | | P[Nle]D[Nle]RC(1)SASVEC(2)AQKC(3)LAAIGSIFGKC(1)MNKKC(2)KC(3) |
| | | | YPR-OH |
| 103 | 107 | 103 | H- |
| | | | P[Nle]E[Nle]RC(1)SASVEC(2)AQKC(3)LAAIGSIFGKC(1)[Nle]NKKC(2)KC |
| | | | (3)YPR-OH |
| 104 | 108 | 104 | H-C(1)SASVEC(2)KQKC(3)LKAIGSIFGKC(1)[Nle]NKKC(2)KC(3)YPR-NH₂ |
| 104 | 109 | 105 | Ac-C(1)SASVEC(2)KQKC(3)LKAIGSIFGKC(1)[Nle]NKKC(2)KC(3)YPR- |
| | | | NH₂ |
| 106 | 111 | 106 | H-RC(1)SASVEC(2)KQKC(3)LKAIGSIFGKC(1)[Nle]NKKC(2)KC(3)YPR- |
| | | | NH₂ |
| 107 | 113 | 107 | Ac-SKC(1)SASVEC(2)KQKC(3)LKAIGSIFGKC(1)[Nle]NKKC(2)KC(3)YPR- |
| | | | NH₂ |
| 108 | 114 | 108 | Ac-LRC(1)SASVEC(2)KQKC(3)LKAIGSIFGKC(1)[Nle]NKKC(2)KC(3)YPR- |
| | | | NH₂ |
| 108 | 115 | 114 | H-LRC(1)SASVEC(2)KQKC(3)LKAIGSIFGKC(1)[Nle]NKKC(2)KC(3)YPR- |
| | | | OH |
| 115 | 122 | 115 | Ac-LRC(1)SASVEC(2)KQKC(3)LAAIGSIFGKC(1)[Nle]NKKC(2)KC(3)YPR- |
| | | | OH |
| 116 | 123 | 116 | Ac-LRC(1)SASVEC(2)KQKC(3)LAAIGSIFGKC(1)MNKKC(2)KC(3)YPR-OH |
| 117 | 124 | 117 | H-C(1)SASVEC(2)KQKC(3)LKAIGSIFGKC(1)MNKKC(2)KC(3)YPR-OH |
| 151 | ShK | — | H-RSCIDTIPKSRCTAFQCKHSMKYRLSFCRKTCGTC-OH |
| 152 | ShK- | — | H-[phosphotyrosyl][8-Amino-3,6- |
| | 186 | | dioxaoctanoyl]RSCIDTIPKSRCTAFQCKHSMKYRLSFCRKTCGTC-NH₂ |
| 153 | Moka1 | — | H-INVKCSLPQQCIKPCKDAGMRFGKCMNKKCRCYS-OH |
| 154 | Vm24 | — | H-AAAISCVGSPECPPKCRAQGCKNGKCMNRKCKCYYC-NH₂ |

### Example 2: Selectivity of Kv1.3 Inhibitor Peptides in Patch Clamp Assay

Selectivity of Kv1.3 inhibitor peptides for Kv1.3 over other potassium ion channels (Kv1.1, Kv1.2 and Kv1.6) was determined using patch clamp assay.

Chinese Hamster Ovary (CHO) cell lines stably expressing exogenous human α-subunits of each potassium ion channel were grown and passaged under standard culture conditions.

The automated, chip-based planar patch clamp device QPatch® was used to quantitate the ionic currents. All recordings were made in the conventional whole-cell configuration after establishment of gigaohm seals. External recording solution contained (150 mM NaCl, 10 mM KCl, 10 mM HEPES, 1 mM MgCl₂, 3 mM CaCl₂, 10 mM Glucose, pH adjusted to 7.4 with NaOH) and Internal recording solution (20 mM KCl, 120 mM KF, 10 mM HEPES, 10 mM EGTA, 5 mM NaATP, pH adjusted to 7.2 with KOH). During experiments 0.1% (v/v) BSA was included as a vehicle in all external recording solutions. Currents were elicited from a holding potential of −80 mV using a voltage protocol, which shifted the voltage to 30 mV for 500 ms every 15 s.

Concentration-response relationships were established by cumulatively applying seven escalating concentrations of test sample to an individual cell with a recording period of 2 min per compound application.

The efficacy was determined as the mean charge for the last three sweeps at the end of each concentration application period from the cursor positions. The percent inhibition for each test dose application period was calculated as the reduction in mean cursor value (charge) relative to the cursor value measured at the end of the vehicle period and used to calculate the IC₅₀ from the concentration response curve. Results are shown in Table 3 below.

TABLE 3

| | Potency IC₅₀ (nM) | | | | Selectivity | | |
|---|---|---|---|---|---|---|---|
| Peptide | Kv1.1 | Kv1.2 | Kv1.3 | Kv1.6 | Kv1.1/Kv1.3 | Kv1.2/Kv1.3 | Kv1.6/Kv1.3 |
| 1 | >3000 | 518 | 0.38 | >3000 | >7900 | 1364 | >7900 |
| 2 | >3000 | 97 | 1.50 | 1810 | >2000 | 65 | 1204 |
| 3 | >3000 | 409 | 0.27 | >3000 | >11300 | 1540 | >11300 |
| 6 | >3000 | 285 | 0.38 | >3000 | >7900 | 747 | >7900 |
| 9 | >3000 | 376 | 0.44 | >3000 | >6800 | 857 | >6800 |
| 13 | >3000 | 228 | 0.11 | >3000 | >27600 | 2093 | >27600 |
| 17 | >3000 | 133 | 0.24 | 2460 | >12600 | 561 | 10360 |
| 18 | >3000 | 66 | 0.42 | 912 | >7200 | 159 | 2182 |
| 19 | >3000 | 256 | 0.24 | >3000 | >12700 | 1080 | >12700 |
| 20 | >3000 | 45 | 0.22 | 435 | >13500 | 202 | 1955 |
| 21 | >3000 | 99 | 0.21 | 2527 | >14300 | 471 | 12045 |
| 22 | >3000 | 57 | 0.22 | 626 | >13700 | 259 | 2851 |
| 23 | >3000 | 181 | 0.29 | >3000 | >10200 | 615 | >10200 |
| 24 | 626 | 28 | 0.15 | 215 | 4280 | 191 | 1472 |
| 26 | >3000 | 67 | 0.09 | 363 | >34200 | 761 | 4144 |
| 27 | >3000 | 43 | 0.26 | >3000 | >11800 | 169 | >11800 |
| 28 | >3000 | 11 | 0.12 | 206 | >25900 | 94 | 1778 |
| 29 | >3000 | 16 | 0.20 | 252 | >15200 | 83 | 1272 |
| 30 | >3000 | 874 | 0.40 | >3000 | >7400 | 2163 | >7400 |
| 31 | >3000 | 666 | 0.14 | >3000 | >22000 | 4897 | >22000 |
| 32 | >3000 | 179 | 0.28 | >3000 | >10800 | 648 | >10800 |
| 33 | | | 0.38 | | | | |
| 35 | >3000 | 100 | 0.42 | 207 | >7100 | 237 | 490 |
| 36 | >3000 | 18 | 0.20 | 192 | >15000 | 88 | 963 |
| 37 | >3000 | 439 | 0.31 | >3000 | >9700 | 1422 | >9700 |
| 38 | >3000 | 338 | 0.19 | >3000 | >16100 | 1816 | >16100 |
| 40 | | | 0.15 | | | | |
| 41 | >3000 | 113 | 0.26 | 1829 | >11600 | 436 | 7073 |
| 42 | >3000 | 939 | 0.18 | >3000 | >17100 | 5362 | >17100 |
| 43 | >3000 | 109 | 0.16 | 316 | >18600 | 674 | 1954 |
| 46 | >3000 | 257 | 0.75 | >3000 | >4000 | 343 | >4000 |
| 47 | >3000 | 302 | 0.32 | 1395 | >9400 | 943 | 4359 |
| 48 | >3000 | 772 | 0.54 | 609 | >5600 | 1438 | 1134 |
| 49 | >3000 | >3000 | 0.37 | >3000 | >8200 | >8200 | >8200 |
| 50 | >3000 | 164 | 1.34 | 946 | >2200 | 123 | 707 |
| 51 | | | 0.27 | | | | |
| 52 | | | 0.25 | | | | |
| 53 | >3000 | 88 | 0.11 | >3000 | >27500 | 803 | >27500 |
| 54 | >3000 | 149 | 0.29 | >3000 | >10300 | 514 | >10300 |
| 55 | >3000 | 291 | 2.41 | >3000 | >1200 | 121 | >1200 |
| 56 | | | 0.12 | | | | |
| 57 | >3000 | 57 | 0.30 | 932 | >9900 | 187 | 3062 |
| 59 | >3000 | 145 | 0.34 | >3000 | >8800 | 426 | >8800 |
| 63 | >3000 | 449 | 0.26 | 2349 | >11600 | 1740 | 9102 |
| 64 | >3000 | 184 | 0.31 | 1231 | >9600 | 587 | 3929 |
| 65 | | | 0.25 | | | | |
| 66 | | | 0.14 | | | | |
| 67 | | | 0.18 | | | | |
| 69 | | | 0.32 | | | | |
| 72 | >3000 | 423 | 0.25 | >3000 | >12200 | 1720 | >12200 |
| 73 | >3000 | 373 | 0.11 | >3000 | >27700 | 3438 | >27700 |
| 77 | | | 0.13 | | | | |
| 78 | >3000 | 122 | 0.28 | >3000 | >10600 | 430 | >10600 |
| 81 | >3000 | 1149 | 0.21 | >3000 | >14600 | 5596 | >14600 |
| 82 | | | 0.16 | | | | |
| 84 | >3000 | 480 | 0.028 | >3000 | >100000 | 17000 | >100000 |
| 85 | >3000 | 624 | 0.63 | 1222 | >4800 | 995 | 1949 |
| 91 | >3000 | 380 | 0.064 | >3000 | >46000 | 6000 | >46000 |
| 97 | >3000 | 1859 | 0.17 | >3000 | >17800 | 11034 | >17800 |
| 98 | >3000 | 2063 | 0.27 | >3000 | >11300 | 7756 | >11300 |
| 99 | >3000 | 1758 | 0.39 | >3000 | >7800 | 4561 | >7800 |
| 100 | >3000 | 1117 | 0.33 | >3000 | >9100 | 3384 | >9100 |
| 101 | >3000 | 311 | 0.18 | >3000 | >16200 | 1689 | >16200 |
| 105 | >3000 | 1968 | 0.35 | >3000 | >8600 | 5630 | >8600 |
| 107 | >3000 | >3000 | 0.55 | >3000 | >5500 | 5500 | >5500 |
| 108 | >3000 | >3000 | 0.43 | >3000 | >7000 | >7000 | >7000 |
| 109 | >3000 | 384 | 0.24 | >3000 | >12400 | 1591 | >12400 |
| 111 | >3000 | 280 | 0.40 | 1302 | >7600 | 708 | 3290 |
| 113 | >3000 | 196 | 1.00 | 1451 | >3000 | 196 | 1451 |
| 114 | >3000 | 122 | 0.35 | 2456 | >8700 | 352 | 7096 |
| 124 | >3000 | 1832 | 2.09 | >3000 | >1400 | 878 | >1400 |
| ShK | 0.0021 | 11 | 0.017 | 0.19 | 0.12 | 644 | 11 |
| ShK-186 | 0.31 | 43 | 0.095 | 0.45 | 4.9 | 682 | 7.4 |
| Moka1 | >3000 | 275 | 9.0 | >3000 | >300 | 31 | >300 |
| Vm24 | 0.097 | 4.7 | 0.15 | 21 | 0.65 | 31 | 139 |

### Example 3a: Inhibitory Activity of Kv1.3 Inhibitor Peptides on Human PBMCs

Human peripheral blood mononuclear cells (PBMCs) were used to assess the effects of Kv1.3 inhibitor peptides on T-cell activation as determined by IL-2 (cytokine) release after stimulation with anti-CD3.

Human PBMCs were obtained from Precision for Medicine (Frederick, MD). Cells from 5 donors were used. Plate-bound anti-CD3 was used to stimulate bulk T cells in the PBMC preparations. Briefly, 96-well plates were coated with anti-CD3 antibody for 2 hrs at 37° C., using 50 μL of a 0.5 μg/mL anti-CD3 solution diluted in 1×PBS. Thereafter the plates were washed twice.

Peptides as shown in Table 4a were diluted in medium (RPMI 1640 with Glutamax-I containing 10% v/v Fetal Bovine Serum, 1% v/v penicillin-streptomycin solution) and added in a volume of 100 μL at concentrations ranging from 0.01 pM to 100 nM (tenfold dilutions). Cyclosporin A (1 ug/ml) and Vm24 peptide (100 nM) were used as positive controls. Finally, 1×10⁵ PBMCs were added to each well in a volume of 100 μL, giving a final volume of 200 μL per well. The plates were incubated for 20-24 hours in a 37° C./5% CO₂ incubator. After centrifugation of the plates, 25 μl supernatant was transferred to IL-2 detection plates (MSD Human IL-2 Tissue Culture Kit, cat #K151AHB-2) and IL-2 was measures as described by the manufacturer (Meso Scale Discovery, Rockville, Maryland, USA).

Results are shown in Table 4a as geometric mean of IC50 values obtained from anti-CD3 stimulated human PBMC assays. All values derive from at least 4 replicates.

TABLE 4a

| | anti-CD3 PBMC/ IL-2 |
|---|---|
| Peptide | IC50 [nM] |
| ShK-186 | 0.07 |
| 17 | 0.05 |
| 18 | 0.4 |
| 21 | 0.1 |
| 36 | 0.1 |

Incubation with anti-CD3 antibody activated hPBMC and addition of reference peptides resulted in dose-dependent reduction in the IL-2 secretion. On average the IC₅₀ values (as calculated from IL-2 release) of the peptides were in the range of 0.05 nM to 0.4 nM. This was comparable to the IC₅₀ observed with ShK186 (IC₅₀ is 0.07 nM) and about 10-100 fold lower than the IC₅₀ of Moka1 which was less potent in inhibiting IL-2 secretion. There is no significant difference between the peptides and ShK186. ShK186 and peptides were all significantly lower than Moka1. Cyclosporine blocked CD3 induced IL-2 release completely in all experiments.

### Example 3b: Inhibitory Activity of Kv1.3 Inhibitor Peptides on Human PBMCs

Human peripheral blood mononuclear cells (PBMCs) were used to assess the effects of Kv1.3 inhibitor peptides on T-cell activation as determined by IL-2 release after stimulation with anti-CD3.

Human PBMCs were obtained from Precision for Medicine (Frederick, MD). Cells from 5 donors were used. Plate-bound anti-CD3 was used to stimulate bulk T cells in the PBMC preparations. Briefly, 96-well plates were coated with anti-CD3 antibody for app. 16 hours at 5° C., using 50 μl of a 1 μg/ml anti-CD3 solution diluted in PBS. Thereafter the plates were washed twice.

Peptides were subsequently diluted in medium (RPMI 1640 with Glutamax-I containing, 10% v/v Fetal Bovine Serum, 1% v/v penicillin-streptomycin Solution) and added in a volume of 50 μl. The peptides indicated in Table 4b were used at concentrations ranging from 0.3 pM to 1000 nM (half log dilutions, starting concentrations varying). Cyclosporin A (1 μg/ml) and Vm24 peptide (100 nM) were used as positive controls.

Finally, 50,000 PBMCs in the same medium were added to each well in a volume of 50 μl, giving a final volume of 100 μl per well. The plates were incubated for 20-24 hours in a 370C/5% CO₂ incubator. After centrifugation of the plates, 25 μl supernatant was transferred to IL-2 detection plates (MSD Human IL-2 Tissue Culture Kit, cat #K151AHB-2) and IL-2 was measures as described by the manufacturer (Meso Scale Discovery, Rockville, Maryland, USA).

Results are shown in Table 4b as geometric mean of IC₅₀ values obtained from anti-CD3 stimulated human PBMC assays. All values derive from at least 6 replicates.

TABLE 4b

| | anti-CD3 PBMC/IL-2 |
|---|---|
| Peptide | IC50 [nM] |
| ShK-186 | 0.04 |
| 1 | 0.09 |
| 3 | 0.06 |
| 24 | 0.06 |
| 42 | 0.03 |
| 49 | 0.07 |

Incubation with anti-CD3 antibody activated hPBMC and addition of Kv1.3 inhibitor peptides resulted in dose-dependent reduction in the IL-2 secretion.

The average IC₅₀ values (as calculated from IL-2 release) of the peptides were in the range of 0.01 nM to 0.09 nM as shown in Table 4b. This was comparable to the IC₅₀ observed with ShK186 (IC₅₀ is 0.05 nM). This assay was performed using different donors than those used for Example 3a, so identical values for the Shk-186 in the two sets of experiments are not expected.

Cyclosporine blocked anti-CD3 induced IL-2 release completely in all experiments.

### Example 4: Inhibitory Activity of Kv1.3 Inhibitor Peptides in Rat Whole Blood

Rat whole blood was used to assess the potency of Kv1.3 inhibitor peptides on T-cell activation as determined by IL-17A release after stimulation with thapsigargin. Addition of thapsigargin results in activation of a signalling cascade ending up in activation of T cell proliferation and cytokine production where the Kv1.3 ion channel plays a key role, so activity of Kv1.3 inhibitors in primary cells can be measured in this experimental system.

Rat whole blood was obtained from healthy, naïve Lewis or Sprague-Dawley rats that were terminally bleed from the heart using Sodium Heparin blood sampling tubes for collection. Peptides were diluted to 4× final testing concentrations in assay buffer (DMEM+GlutaMAX), GlutaMAX is a medium comprising 3.97 mM L-alanine-L-glutamine (Gibco Cat #61965026) supplemented with 25 mM HEPES buffer, 1 mM Sodium Pyruvate, 100 units/ml Penicillin, 100 μg/ml Streptomycin and 0.05% Casein from bovine milk (Sigma-Aldrich)) and 25 μl was added to wells of a 96 well plate. Then 50 μl whole rat blood was added and incubated for minimum 5 minutes at room temperature to allow compound binding. Then 25 μl 40 μM thapsigargin diluted in assay buffer was added to all wells of the assay plates to activate the cells, followed by incubation for 24 Hr at 37° C./5% CO₂ in a humidified box. The assay plates were centrifuged for 10 min at 300 g at 4° C. and the supernatants were transferred to new plates. The concentrations of IL-17A released to the supernatants were measured using a Rat IL-17A ELISA Kit (Abcam Cat #ab214028) as recommended by the manufacturer. Samples were diluted 2.5-fold by transferring 20 μl of the supernatants to wells on ELISA plates containing 30 μl buffer 75BS from the detection kit.

Data from peptides eliciting an inhibition of IL-17A were normalised relative to full thapsigargin activation (no inhibitor added) and no activation controls (addition of assay buffer instead of thapsigargin) to calculate the IC₅₀ from the concentration response curve.

Results are shown in Table 5, expressed as IC₅₀, with standard deviation (IC₅₀_SD). All values are derived from at least 2 replicates. The biological effects ex vivo show a correlation with the potency of the peptides.

TABLE 5

| Peptide | IC₅₀ | IC₅₀_{_{—}}SD |
|---|---|---|
| 1 | 0.17 | 0.036 |
| 3 | 1.0 | 0.31 |
| 18 | 0.88 | 0.32 |
| 19 | 3.2 | 0.017 |
| 20 | 0.56 | 0.2 |
| 21 | 1.2 | 0.72 |
| 22 | 0.82 | 0.81 |
| 23 | 2.8 | 2.5 |
| 24 | 0.57 | 0.24 |
| 27 | 0.61 | 0.33 |
| 28 | 0.68 | 0.47 |
| 29 | 0.53 | 0.21 |
| 30 | 0.76 | 0.23 |
| 31 | 0.41 | 0.021 |
| 32 | 0.86 | 0.31 |
| 33 | 0.75 | 0.24 |
| 34 | 0.70 | 0.40 |
| 35 | 2.1 | 0.89 |
| 36 | 2.5 | 2.1 |
| 37 | 1.2 | 0.7 |
| 38 | 0.77 | 0.56 |
| 39 | 2.8 | 1.8 |
| 42 | 0.98 | 0.60 |
| 43 | 0.91 | 0.26 |
| 44 | 0.99 | 0.24 |
| 45 | 1.1 | 0.47 |
| 46 | 1.4 | 0.64 |
| 47 | 0.79 | 0.28 |
| 48 | 0.83 | 0.4 |
| 49 | 0.63 | 0.039 |
| 50 | 1.0 | 0.65 |
| 53 | 0.69 | 0.21 |
| 63 | 0.44 | 0.080 |
| 64 | 0.68 | 0.19 |
| 65 | 1.2 | 0.71 |
| 66 | 1.1 | 0.011 |
| 67 | 0.47 | 0.35 |
| 68 | 0.52 | 0.059 |
| 69 | 0.98 | 0.076 |
| 70 | 0.93 | 0.72 |
| 71 | 2.7 | 0.54 |
| 72 | 0.79 | 0.15 |
| 73 | 0.30 | 0.11 |
| 74 | 0.47 | 0.086 |
| 75 | 0.52 | 0.019 |
| 76 | 1.6 | 1.1 |
| 77 | 0.86 | 0.13 |
| 78 | 0.55 | 0.25 |
| 80 | 0.39 | 0.28 |
| 85 | 0.81 | 0.13 |
| 86 | 1.1 | 1.2 |
| 87 | 0.69 | 0.38 |
| 88 | 1.4 | 1.1 |
| 89 | 1.1 | 1.3 |
| 90 | 0.73 | 0.28 |
| 94 | 0.34 | 0.07 |
| 95 | 1.3 | 1.0 |
| 96 | 0.54 | 0.31 |
| 97 | 0.55 | 0.42 |
| 98 | 1.2 | 0.57 |
| 99 | 1.8 | 1.2 |
| 100 | 0.43 | 0.42 |
| 101 | 0.34 | 0.21 |
| 103 | 0.5 | 0.37 |
| 104 | 0.87 | 0.84 |
| 105 | 0.61 | 0.34 |
| 106 | 0.32 | 0.22 |
| 107 | 1.2 | 1.5 |
| 108 | 1.6 | 0.94 |
| 109 | 0.88 | 0.011 |
| 111 | 0.52 | 0.34 |
| 113 | 0.36 | 0.13 |
| 114 | 0.37 | 0.049 |
| 115 | 2.1 | 2.5 |
| 122 | 0.9 | 0.3 |
| 123 | 0.36 | 0.24 |
| 124 | 1.9 | 1 |

### Example 5: Inhibitory Activity of Kv1.3 Inhibitor Peptides in Human Whole Blood

Human whole blood was used to assess the potency of Kv1.3 inhibitor peptides on T-cell activation as determined by release of the cytokines IFN-g, IL-2 and IL-17A after stimulation with thapsigargin. Addition of thapsigargin activates a signalling cascade resulting in T cell proliferation and cytokine production, in which the Kv1.3 ion channel plays a key role. Activity of Kv1.3 inhibitors in human primary T cells can thus be measured in this experimental system.

Human whole blood was obtained from healthy blood donors and was collected after informed consent using Sodium Heparin blood sampling tubes (Becton, Dickinson and Company (BD), Cat #367876). Test peptides were diluted to 4× final testing concentrations in assay buffer (Dulbecco's Modified Eagle's medium (DMEM) with high glucose and GlutaMAX (3.97 mM L-alanine-L-glutamine) (Gibco Cat #61965026) supplemented with 25 mM HEPES buffer, 1 mM Sodium Pyruvate, 100 units/ml Penicillin, 100 μg/ml Streptomycin and 0.05% Casein from bovine milk (Sigma-Aldrich Cat #C4765)) and 25 μl was added to wells of a 96 well tissue culture plate. Then 50 μl human whole blood was added and incubated for minimum 5 minutes at room temperature to allow compound binding. Then 25 μl of 40 μM thapsigargin diluted in assay buffer was added to all wells of the assay plates to activate the cells, followed by incubation for 24 Hr at 37° C./5% CO₂ in a humidified box. The assay plates were centrifuged for 10 min at 300 g at 4° C. and the supernatants were transferred to new plates. The concentrations of IFN-g, IL-2 and IL-17A released to the supernatants were measured using a tri-plex human cytokine detection kit (MSD Human U-Plex IFN-g, IL-2 and IL-17A kit; Meso Scale Discovery Cat #K15067L-2) and all three cytokines were measured using a MSD MESO QuickPlex SQ 120 instrument as described by the manufacturer (Meso Scale Discovery, Rockville, Maryland, USA).

To determine the potency of inhibition for the compounds, the cytokine concentration data were fitted using a three parameter logistic dose response model based on the formula Y=Bottom+(Top−Bottom)/(1+IC50/X), where Y is measured cytokine concentrations, X is compound concentrations and Top, bottom and IC₅₀ are parameters fitted using the software Graphpad Prism version 5.04. The IC₅₀ value calculated from the concentration response curve represents the compound concentration that gives an inhibition response halfway between the basal (Bottom) response and the maximal (Top) response.

Results are shown in Table 6, expressed as IC₅₀. All values are derived from at least 2 replicate experiments. All of the tested Kv1.3 blockers were able to inhibit cytokine production from human T cells present in human whole blood that was stimulated with thapsigargin.

TABLE 6

| | Average IC₅₀ [nM] | | |
|---|---|---|---|
| Peptide | IFN-g | IL-2 | IL-17A |
| 3 | 0.058 | 0.022 | 0.044 |
| 42 | 0.046 | 0.030 | 0.058 |
| 53 | 0.11 | 0.083 | 0.26 |
| 100 | 0.11 | 0.095 | 0.15 |
| 108 | 0.081 | 0.12 | 0.23 |

### Example 6: Pharmacokinetic Characterisation of Kv1.3 Inhibitors

Sprague Dawley or Wistar rats (males with a body weight of approximately 250-350 g) were given a single subcutaneous (s.c.) injection of each peptide to be tested.

Following s.c. administration of the selected peptides (dose 70 nmol/kg, dosing volume either 2 or 5 mL/kg), blood samples were drawn at 15 min, 30 min, 45 min, 60 min, 90 min, 2 h, 3 h, 4 h post-dose. At each sampling time point, samples from the rats were drawn by sublingual bleeding or by tail cut. After last sampling the rats were sacrificed by O₂/CO₂ anaesthesia. The dosing vehicle was 10 mM phosphate, 0.8% NaCl, 0.05% Polysorbate 20 (pH 6.0).

Plasma samples were analyzed after solid phase extraction (SPE) by liquid chromatography mass spectrometry (LC-MS/MS). Mean plasma concentrations were used for calculation of the pharmacokinetic parameters using the non-compartmental approach in Phoenix WinNonlin 6.4 or a later version. Plasma terminal elimination half-life (T %) was determined as ln(2)/λz where λz is the magnitude of the slope of the log linear regression of the log concentration versus time profile during the terminal phase. AUC_{inf} is the area under the plasma concentration−time curve extrapolated to infinity (AUC_{inf}=AUCₗₐₛₜ+Cₗₐₛₜ/λz, where Cₗₐₛₜ is the last observed plasma concentration). Cmax is the maximum observed concentration, occurring at Tmax.

Results for some exemplary peptides are shown in Table 7.

TABLE 7

| | Dose | AUC_{INF} | Cmax | T_{1/2} |
|---|---|---|---|---|
| Ptd No. | nmol/kg | (hr*nmol/L) | (nmol/L) | (hr) |
| 3 | 70 | 83.9 | 36.9 | 0.99 |
| 6 | 70 | 72.4 | 48.4 | 0.93 |
| 9 | 70 | 98.3 | 76.9 | 0.62 |
| 31 | 70 | 46.7 | 33.3 | 0.74 |
| 53 | 70 | 48.5 | 31.2 | 0.89 |
| 54 | 70 | 31.1 | 50.0 | 0.87 |
| 98 | 70 | 85.0 | 46.5 | 1.09 |
| 100 | 70 | 112.0 | 52.6 | 1.04 |
| 105 | 70 | 131.8 | 58.6 | 0.91 |
| 107 | 70 | 176.6 | 68.4 | 1.36 |

### Example 7: Effect of Treatment with Kv1.3 Inhibitor Peptides in Keyhole Limpet Hemocyanin (KLH) Ear Inflammation Model in Rats

A classical delayed-type hypersensitivity (DTH) reaction was elicited in one ear of rats. Briefly, male Lewis rats aged 8-10 weeks were immunized on day −7 with 200 μL keyhole limpet hemocyanin (KLH) (from Sigma, cat.no. H7017) (4 mg/mL) emulsified in complete Freund's adjuvant (CFA) (Difco, cat.no. 263810) subcutaneously (SC) at the base of the tail. On day 0 the rats were challenged intradermally with 40 μL KLH/NaCl 0.9% (2 mg/mL) in the left ear. After the ear challenge the rats develop a T-cell dependent inflammation in the left KLH challenged ear. The right ear remains uninflamed and serves as control.

The ability of treatment with Kv1.3 inhibitor peptides to reduce the DTH ear swelling response was investigated by comparing the response in rats (n=8-10/gr) treated with vehicle to that of rats treated with Kv1.3 inhibitor peptides. Vehicle or peptide dissolved in vehicle was administered SC (2 mL/kg) 24 hrs prior to KLH ear challenge. The test dose of peptide was 50, 70 or 100 nmol/kg. Test vehicle was 10 mM phosphate, 0.8% w/v NaCl, 0.05% w/v polysorbate20, pH 6. Cyclosporine (CsA) was included as positive study control in all experiments. Cyclosporine (Sandimmune Neooral® 100 mg/mL oral solution, Novartis) was administered per os (10 mg/kg) one hour prior to KLH ear challenge and again 6 hours after KLH ear challenge.

As primary read-out of efficacy, the Area Under Curve (AUC) of A ear thickness (mm) was calculated for each animal from 0-48 hours post induction of the ear DTH reaction, where the change (D) was calculated as: Left ear thickness-right ear thickness. These results were then used to calculate % inhibition of ear thickness by Kv1.3 inhibitor treatment: % inhibition: ((1−(individual A AUC Kv1.3 inhibitor/average ΔAUC vehicle group))×100. Results were calculated as % inhibition+/−standard deviation (SD), and are shown in Table 8 and Table 9.

TABLE 8

| | Dose | | | | |
|---|---|---|---|---|---|
| Exp | (nmol/kg) | Ptd 3 | Ptd 42 | Ptd 53 | CsA* |
| #1 | 70 | 38.8 (+/−9.7) | 46.9 (+/−10.9) | — | 71.4 (+/−4.8) |
| #2 | 70 | 25.0 (+/−12.5) | 27.7 (+/−11.5) | — | 76.8 (+/−13.4) |
| #3 | 50 | 37.3 (+/−13.8) | 22.2 (+/−11.1) | — | 65.1 (+/−4.5) |
| #4 | 100 | — | 41.9 (+/−12.5) | 25.1 (+/−6.3) | 63.4 (+/−5.9) |

TABLE 9

| | Dose | | | | | |
|---|---|---|---|---|---|---|
| Exp. | (nmol/kg) | Ptd 98 | Ptd 100 | Ptd 105 | Ptd 107 | CsA |
| #5 | 100 | 39.6 | — | — | — | 67.1 |
| | | (+/−8.3) | | | | (+/−7.9) |
| #6 | 100 | — | 42.5 | 44.5 | — | 55.3 |
| | | | (+/−14.0) | (+/−5.2) | | (+/−5.5) |
| #7 | 100 | — | — | — | 48.9 | 67.9 |
| | | | | | (+/−8.6) | (+/−5.4) |
| #8 | 100 | — | 37.7 | — | — | 57.8 |
| | | | (+/−10.0) | | | (+/−2.8) |

### Example 8: Long-Lasting Effect of Treatment with a Kv1.3 Inhibitor Peptide in KLH-Induced Delayed Type Hypersensitivity (DTH) Model

### Animal Care Committee

The animal care facility employed is accredited by AAALAC. Female Lewis rats, weighing 180±20 g at delivery were used for this study. Following arrival in the animal facility, all animals were subjected to a general health evaluation. An acclimation period of 1 day was allowed before the beginning of the study.

### Housing Environment

The animals were housed under standardized environmental conditions. The rats were housed in open-topped cages, 6 animals per cage. A standard certified commercial rodent diet was provided ad libitum. Tap water was provided ad libitum at all times. It is considered that there are no known contaminants in the diet and water that would interfere with the objectives of the study. Each cage was identified for the corresponding group, indicating the treatment and the identity of the animals housed in the cage.

The animal room was maintained at a controlled temperature of 20-24° C. and a relative humidity of 30-70%. A controlled lighting system assured 12 hours light, 12 hours dark per day to the animals. Adequate ventilation of 15 air changes per hour was maintained.

### Study Details

This study was performed to investigate the length of effect of a Kv1.3 inhibitor (peptide 100) on ear inflammation in the KLH-induced DTH model in rats.

Healthy male Lewis rats aged 8-9 weeks were immunized on day 0 with 200 μL keyhole limpet hemocyanin (KLH) (from Sigma, cat.no. H7017) (4 mg/mL) emulsified in complete Freund's adjuvant (CFA) (Difco, cat.no. 263810) subcutaneously (SC) at the base of the tail.

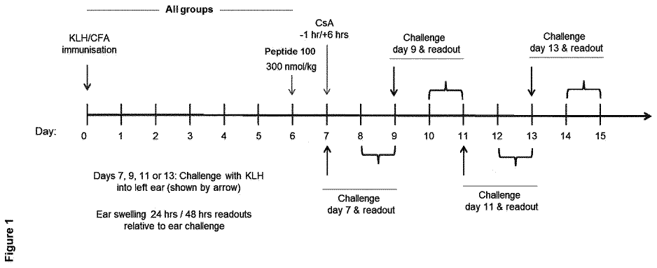

To elicit a KLH-specific T cell-mediated local inflammation, rats were challenged intradermally at indicated timepoints (days 7, 9, 11 or 13 post-immunisation) with 40 μL KLH/NaCl 0.9% (2 mg/mL) in the left ear. Ear edemas were measured after 24-48 hrs and the right ear that was left untreated served as control. A schematic overview of the protocol is depicted in FIG. 1.

The ability of Kv1.3 inhibitor treatment to reduce the DTH ear swelling response was investigated by comparing the response in rats (n=8/group) treated with vehicle to that of rats treated with peptide 100. Vehicle or peptide 100 dissolved in vehicle was administered SC (2 mL/kg) on day 6. The test dose of Kv1.3 inhibitor was 300 nmol/kg. Test vehicle was 10 mM phosphate, 0.8% w/v NaCl, 0.05% w/v polysorbate20, pH 6. Cyclosporine A (CsA) was included as positive study control. Cyclosporine (Sandimmune Neoral® 100 mg/mL oral solution, Novartis) was administered on day 7 per os (10 mg/kg) one hour prior to KLH ear challenge and again 6 hours after KLH ear challenge.

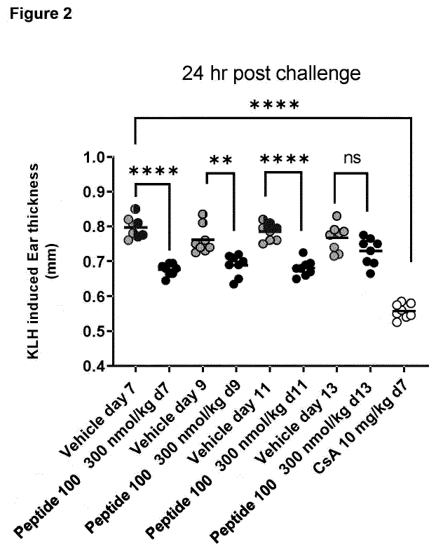

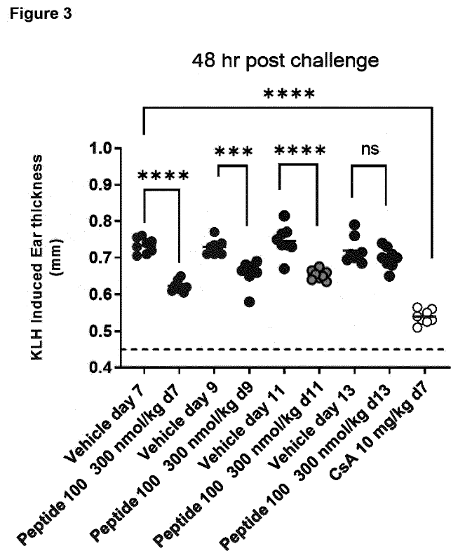

As readout of efficacy, the thickness of the induced ear 24 hrs and 48 hrs after respective ear challenge was measured and compared to vehicle control. Ear thickness 24 hrs after challenge is shown in Table 10 and FIG. 2 and ear thickness 48 hrs after challenge is shown in Table 11 and FIG. 3. The results are also summarised below.

For animals challenged on day 7 (i.e. 1 day after treatment), peptide 100 and CsA significantly reduced ear swelling compared to vehicle control when ear swelling was measured 24 hrs after challenge (i.e. 2 days after treatment) (peptide 100: 0.676±0.017 mm; vehicle: 0.796±0.03 mm; CsA: 0.557±0.021 mm; p<0.0001) and when ear swelling was measured 48 hrs after challenge (i.e. 3 days after treatment) (peptide 100: 0.623±0.015 mm; vehicle: 0.733±0.02 mm; CsA: 0.539±0.018 mm; p<0.0001).

Ear swelling was significantly reduced compared to vehicle control when rats were ear challenged on day 9 (i.e. 3 days after treatment) when ear swelling was measured 24 hrs after challenge (i.e. 4 days after treatment) (peptide 100: 0.689±0.031 mm; vehicle: 0.761±0.04 mm; p=0.0011) and when ear swelling was measured 48 hrs after challenge (i.e. 5 days after treatment) (peptide 100: 0.659±0.034 mm; vehicle: 0.729±0.02 mm; p=0.0002)

Ear swelling was significantly reduced compared to vehicle control when rats were ear challenged on day 11 (i.e. 5 days after treatment) when ear swelling was measured 24 hrs after challenge (i.e. 6 days after treatment) (peptide 100: 0.681±0.023 mm; vehicle: 0.784±0.025 mm; p<0.0001) and when ear swelling was measured 48 hrs after challenge (i.e. 7 days after treatment) (peptide 100: 0.653±0.013 mm; vehicle: 0.746±0.41 mm; p<0.0001).

For ear challenge on day 13 (i.e. 7 days after treatment), the reduction was not significant when ear swelling was measured 24 hrs after challenge (i.e. 8 days after treatment) (peptide 100: 0.729±0.038 mm; vehicle: 0.767±0.039 mm; p=0.0696) and when ear swelling was measured 48 hrs after challenge (i.e. 9 days after treatment) (peptide 100: 0.699±0.028 mm; vehicle: 0.720±0.036 mm; p=0.2256).

In conclusion, peptide 100 administered on day 6 reduced KLH-induced ear swelling after challenges on days 7, 9 and 11 when measured 24 hours post-challenge (i.e. a reduction in ear swelling was observed on days 2, 4 and 6 post-treatment) and when measured 48 hours post-challenge (i.e. a reduction in ear swelling was measured on days 3, 5 and 7 post-treatment).

TABLE 10
Mean ear thickness (mm) ± SD of KLH-
induced ear measured 24 hrs post-challenge

| | Group | | | | | | | | |
|---|---|---|---|---|---|---|---|---|---|
| | 1 | 2 | 3 | 4 | 5 | 6 | 7 | 8 | 9 |
| | Treatment | | | | | | | | |
| | | Ptd | | Ptd | | Ptd | | Ptd | |
| | Veh. | 100 | Veh. | 100 | Veh. | 100 | Veh. | 100 | CsA |
| | Day measured (post-treatment) | | | | | | | | |
| | 2 | | 4 | | 6 | | 8 | | 2 |
| Mean | 0.796 | 0.676 | 0.761 | 0.689 | 0.784 | 0.681 | 0.767 | 0.729 | 0.557 |
| Std Dev. | 0.030 | 0.017 | 0.040 | 0.031 | 0.025 | 0.023 | 0.039 | 0.038 | 0.021 |

| | | | | | |
|---|---|---|---|---|---|
| p-value | p < 0.0001 | p = 0.0011 | p < 0.0001 | p = 0.0696 | p < 0.0001 |

TABLE 11
Mean ear thickness (mm) ± SD of KLH-
induced ear measured 48 hrs post-challenge

| | Groups | | | | | | | | |
|---|---|---|---|---|---|---|---|---|---|
| | 1 | 2 | 3 | 4 | 5 | 6 | 7 | 8 | 9 |
| | Treatment | | | | | | | | |
| | | Ptd | | Ptd | | Ptd | | Ptd | |
| | Veh. | 100 | Veh. | 100 | Veh. | 100 | Veh. | 100 | CsA |
| | Day measured (post-treatment) | | | | | | | | |
| | 3 | | 5 | | 7 | | 9 | | 3 |
| Mean | 0.733 | 0.623 | 0.729 | 0.659 | 0.746 | 0.653 | 0.720 | 0.699 | 0.539 |
| Std. Dev. | 0.020 | 0.015 | 0.020 | 0.034 | 0.041 | 0.013 | 0.036 | 0.028 | 0.018 |

| | | | | | |
|---|---|---|---|---|---|
| p-value | p < 0.0001 | p = 0.0002 | p < 0.0001 | p = 0.2256 | p < 0.0001 |

### Example 9: Different Dosing Regimens of a Kv1.3 Inhibitor in a Rat Collagen-Induced Arthritis (CIA) Model

### Peptide and Vehicle Formulations

Vehicle: 10 mM phosphate pH 6+0.8% NaCl+0.05% polysorbate 20

Peptide: Peptide 100 is formulated in vehicle at 50 nmol/mL. Dosing volume was individually adjusted according to the body weight of each animal to reach the target dose of peptide 100 of 100 nmol/kg.

### Animals

The animal care facility employed is accredited by AAALAC. Female Lewis rats, weighing 180±20 g at delivery were used for this study. Following arrival in the animal facility, all animals were subjected to a general health evaluation. An acclimation period of 1 day was allowed before the beginning of the study.

The animals were housed under standardized environmental conditions. The rats were housed in open-topped cages, 6 animals per cage. A standard certified commercial rodent diet was provided ad libitum. Tap water was provided ad libitum at all times. It is considered that there are no known contaminants in the diet and water that would interfere with the objectives of the study. Each cage was identified for the corresponding group, indicating the treatment and the identity of the animals housed in the cage.

The animal room was maintained at a controlled temperature of 20-24° C. and a relative humidity of 30-70%. A controlled lighting system assured 12 hours light, 12 hours dark per day to the animals. Adequate ventilation of 15 air changes per hour was maintained.

### Immunization of Rats

Animals were grouped into 9 each based on their body weight. All animals were challenged with porcine type-II collagen with incomplete Freund's adjuvant on day 1 (0.2 mg/0.2 mL/rat, subcutaneously at the base of the tail) and boosted on day 7 (0.1 mg/0.1 mL/rat, s.c.). In the study dexamethasone given daily PO at 0.3 mg/kg was used as a positive control.

### Dosing of Test Peptide and Vehicle

From day 12 to day 30, peptide 100 was administered as a single bolus either daily (QD), every third day (Q3D) or every fifth day (Q5D) subcutaneously into the flank. The days the animals did not receive the peptide, the animals were dosed with vehicle only. The peptide and vehicle were dosed at a volume of 2 mL/kg. Dosing volume was individually adjusted according to the body weight of each animal to reach the target dose of formulation 1 of 100 nmol/kg.

### Disease Score

Disease was assessed by using a qualitative severity score system (see below, maximum score of 16) on days 1, 7, 10, 12 (before dosing), 13, 14, 15, 16, 17, 18, 19, 20, 21, 22, 24, 26 and 28 before peptide treatment and one hour after treatment on day 30.

The disease severity score is based on the following:

| Score | Condition |
|---|---|
| 0 | Normal |
| 1 | Mild, but definite redness and swelling of the ankle or wrist, |
| | or apparent redness and swelling limited to individual digits, |
| | regardless of the number of affected digits |
| 2 | Moderate redness and swelling of ankle of wrist |
| 3 | Severe redness and swelling of the entire paw including digits |
| 4 | Maximally inflamed limb with involvement of multiple joints |

The ability of Kv1.3 inhibitor treatment to reduce chronic inflammation in arthritis was investigated by comparing the response in rats (n=9 animals/group) treated with vehicle to that of rats treated with peptide 100 daily, every third day or every fifth day. The severity of arthritis was determined by summation of the disease score on all four paws from day 12 to day 30 for each animal. These results are shown in Table 12. Treatment with peptide 100 at all dosing regimens resulted in a reduction of the mean clinical score with all dosing regimens.

TABLE 12

| | Vehicle | | Ptd 100 QD | | Ptd 100 Q3D | | Ptd 100 Q5D | |
|---|---|---|---|---|---|---|---|---|
| Day | Mean | SD | Mean | SD | Mean | SD | Mean | SD |
| 12 | 0.8 | 1.0 | 0.8 | 0.8 | 0.8 | 0.8 | 2.4 | 4.2 |
| 13 | 2.2 | 1.4 | 2.1 | 0.6 | 2.0 | 1.7 | 3.3 | 4.3 |
| 14 | 4.7 | 1.8 | 4.3 | 1.2 | 4.1 | 1.8 | 4.6 | 4.4 |
| 15 | 5.3 | 1.3 | 4.7 | 1.0 | 4.4 | 2.2 | 4.9 | 4.8 |
| 16 | 7.3 | 1.0 | 5.8 | 1.0 | 5.0 | 1.9 | 5.4 | 5.3 |
| 17 | 7.7 | 1.4 | 6.0 | 1.0 | 5.6 | 1.7 | 5.8 | 5.6 |
| 18 | 8.7 | 1.2 | 6.6 | 1.0 | 6.4 | 1.4 | 6.2 | 6.1 |
| 19 | 8.9 | 1.6 | 6.7 | 1.0 | 6.6 | 1.2 | 6.4 | 6.4 |
| 20 | 9.8 | 1.7 | 7.0 | 1.3 | 7.2 | 1.4 | 6.9 | 6.7 |
| 21 | 9.6 | 2.1 | 7.1 | 1.1 | 7.3 | 1.4 | 7.1 | 7.0 |
| 22 | 10.2 | 2.8 | 7.1 | 0.9 | 8.0 | 2.0 | 7.6 | 7.2 |
| 24 | 10.9 | 3.5 | 6.9 | 1.2 | 8.2 | 2.5 | 8.2 | 7.8 |
| 26 | 11.4 | 3.3 | 7.0 | 1.2 | 8.4 | 2.4 | 8.5 | 8.5 |
| 28 | 10.6 | 2.5 | 7.8 | 2.3 | 9.0 | 2.7 | 9.0 | 9.0 |
| 30 | 10.6 | 2.0 | 8.4 | 2.5 | 10.0 | 2.5 | 9.4 | 9.8 |

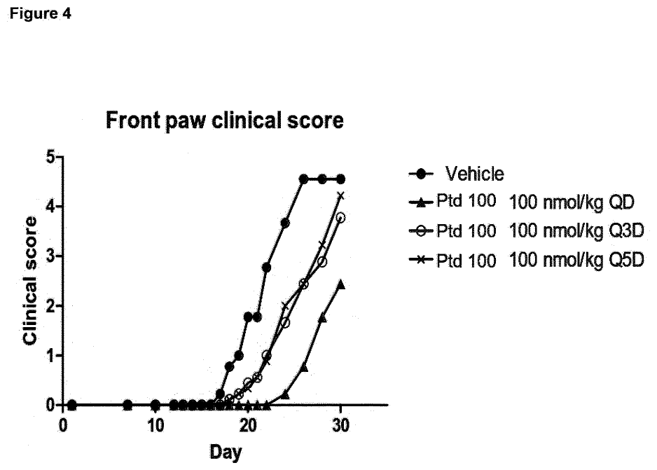

The mean clinical score of the front paw was analysed separately and is shown in FIG. 4. All three dosing regimens (daily, every third day and every fifth day) were able to delay disease in the front paws.

### Example 10: Determination of Maximal Effective Dose of Treatment with a Kv1.3 Inhibitor Peptide in KLH-Induced Delayed Type Hypersensitivity (DTH) Model

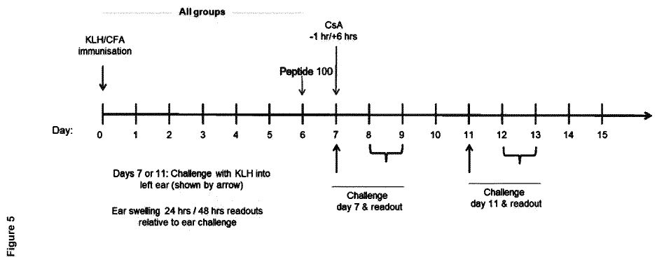

This study was conducted as described in example 8 with the difference that the rats were only challenged intradermally at days 7 or 11 post-immunisation (i.e. day 1 and day 5 after treatment) with 40 μL KLH/NaCl 0.9% (2 mg/mL) in the left ear. A schematic overview of the protocol is depicted in FIG. 5.

The doses of Kv1.3 inhibitor treatment able to reduce the DTH ear swelling response was investigated by comparing the response in rats (n=8/group) treated with vehicle to that of rats treated with different doses of peptide 100. Vehicle or peptide 100 dissolved in vehicle was administered SC (2 mL/kg) on day 6 post-immunisation. The test dose of Kv1.3 inhibitor was 10, 100, 300 or 700 nmol/kg. Test vehicle was 10 mM phosphate, 0.8% w/v NaCl, 0.05% w/v polysorbate20, pH 6.

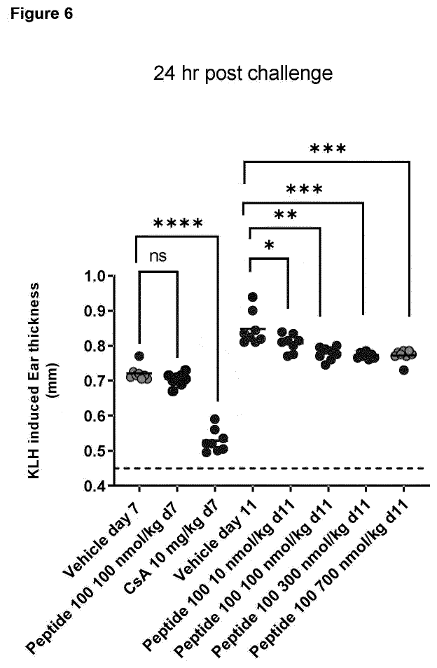

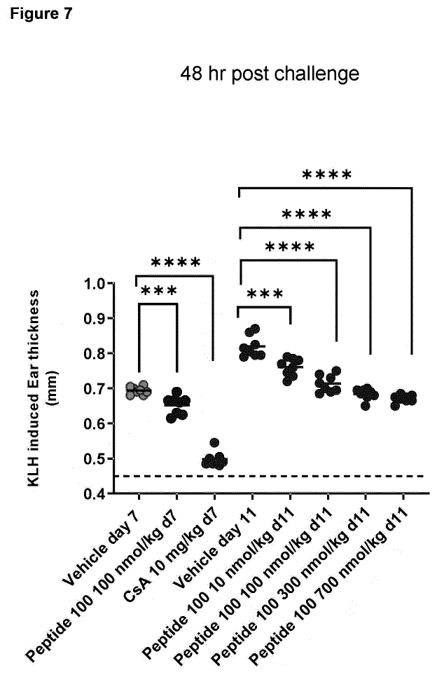

As readout of efficacy, the thickness of the induced ear 24 hrs and 48 hrs after respective ear challenge was measured and compared to vehicle control. Ear thickness 24 hrs after challenge is shown in Table 13 and FIG. 6 and ear thickness 48 hrs after challenge is shown in Table 14 and FIG. 7. The results are also summarised below.

For animals challenged on day 7 (i.e. 1 day after treatment), ear swelling was reduced for CsA compared to vehicle control when ear swelling was measured 24 hrs after challenge (i.e. 2 days after treatment) (peptide 100: 0.704±0.018 mm, p=0.1216; vehicle: 0.721±0.021 mm; CsA: 0.528±0.032 mm, p<0.0001) and when ear swelling was measured 48 hrs after challenge (i.e. 3 days after treatment) (peptide 100: 0.652±0.026 mm, p=0.0008; vehicle: 0.694±0.011 mm; CsA: 0.497±0.021 mm, p<0.0001).

Ear swelling was significantly reduced compared to vehicle control when rats were ear challenged on day 11 (i.e. 5 days after treatment) with doses of peptide 100 of 10, 100, 300 or 700 nmol/kg when ear swelling was measured 24 hrs after challenge (i.e. 6 days after treatment) (peptide 100 at 10 nmol/kg: 0.807±0.025 mm, p=0.0495; 100 nmol/kg: 0.777±0.019 mm, p=0.0015; 300 nmol/kg: 0.772±0.009 mm, p=0.0005; 700 nmol/kg: 0.771±0.018 mm, p=0.0007; vehicle: 0.848±0.047 mm) and when ear swelling was measured 48 hrs after challenge (i.e. 7 days after treatment) (peptide 100 at 10 nmol/kg: 0.761±0.025 mm, p=0.0009; 100 nmol/kg: 0.713±0.034 mm, p<0.0001; 300 nmol/kg: 0.684±0.016 mm, p<0.0001; 700 nmol/kg: 0.671±0.011 mm, p<0.0001; vehicle: 0.819±0.03 mm).

In conclusion, with a dose of 300 nmol/kg of peptide 100, a maximal effect is obtained in the KLH-induced DTH model.

TABLE 13
Mean ear thickness (mm) ± SD of KLH-induced ear measured 24 hrs post-challenge

| | Groups | | | | | | | |
|---|---|---|---|---|---|---|---|---|
| | 1 | 2 | 3 | 4 | 5 | 6 | 7 | 8 |
| | Treatment | | | | | | | |
| | | Ptd 100 | | | Ptd 100 | Ptd 100 | Ptd 100 | Ptd 100 |
| | | 100 | | | 10 | 100 | 300 | 700 |
| | Veh. | nmol/kg | CsA | Veh. | nmol/kg | nmol/kg | nmol/kg | nmol/kg |
| | Day measured (post-treatment) | | | | | | | |
| | 2 | 2 | 2 | 6 | 6 | 6 | 6 | 6 |
| Mean | 0.721 | 0.704 | 0.528 | 0.848 | 0.807 | 0.777 | 0.772 | 0.771 |
| Std. Dev. | 0.021 | 0.018 | 0.032 | 0.047 | 0.025 | 0.019 | 0.009 | 0.018 |
| p-value | | p = 0.1216 | p < 0.0001 | | p = 0.0495 | p = 0.0015 | p = 0.0005 | p = 0.0007 |

TABLE 14
Mean ear thickness (mm) ± SD of KLH-induced ear measured 48 hrs post-challenge

| | Groups | | | | | | | |
|---|---|---|---|---|---|---|---|---|
| | 1 | 2 | 3 | 4 | 5 | 6 | 7 | 8 |
| | Treatment | | | | | | | |
| | | Ptd 100 | | | Ptd 100 | Ptd 100 | Ptd 100 | Ptd 100 |
| | | 100 | | | 10 | 100 | 300 | 700 |
| | Veh. | nmol/kg | CsA | Veh. | nmol/kg | nmol/kg | nmol/kg | nmol/kg |
| | Day measured (post-treatment) | | | | | | | |
| | 3 | 3 | 3 | 7 | 7 | 7 | 7 | 7 |
| Mean | 0.694 | 0.652 | 0.497 | 0.819 | 0.761 | 0.713 | 0.684 | 0.671 |
| Std. Dev. | 0.011 | 0.026 | 0.021 | 0.03 | 0.025 | 0.024 | 0.016 | 0.011 |
| p-value | | p = 0.0008 | p < 0.0001 | | p = 0.0009 | p < 0.0001 | p < 0.0001 | p < 0.0001 |

### Example 11: Determination of Minimal Effective Dose of Treatment with a Kv1.3 Inhibitor Peptide in KLH-Induced Delayed Type Hypersensitivity (DTH) Model

This study was conducted as described in example 8 with the difference that the rats were only challenged intradermally at days 7 or 11 post-immunisation (i.e. day 1 and day 5 after treatment) with 40 μL KLH/NaCl 0.9% (2 mg/mL) in the left ear. A schematic overview of the protocol is depicted in FIG. 5.

The doses of Kv1.3 inhibitor treatment able to reduce the DTH ear swelling response was investigated by comparing the response in rats (n=8/group) treated with vehicle to that of rats treated with different doses of peptide 100. Vehicle or peptide 100 dissolved in vehicle was administered SC (2 mL/kg) on day 6. The test dose of Kv1.3 inhibitor was 1, 3, 10, 30 or 100 nmol/kg. Test vehicle was 10 mM phosphate, 0.8% w/v NaCl, 0.05% w/v polysorbate20, pH 6.

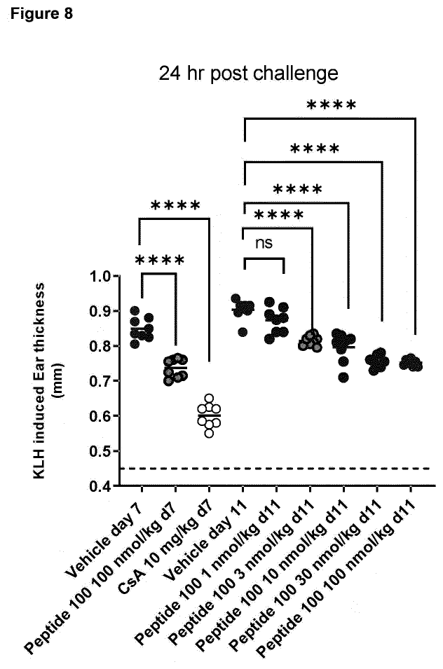

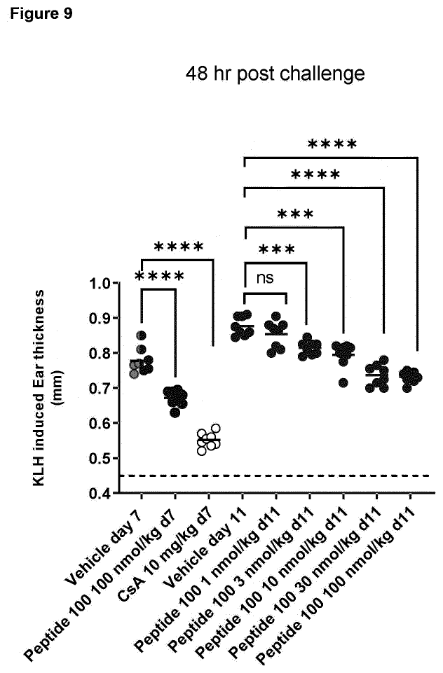

As readout of efficacy, the thickness of the induced ear 24 hrs and 48 hrs after respective ear challenge was measured and compared to vehicle control. Ear thickness 24 hrs after challenge is shown in Table 15 and FIG. 8 and ear thickness 48 hrs after challenge is shown in Table 16 and FIG. 9. The results are also summarised below.

For animals challenged on day 7 (i.e. 1 day after treatment), ear swelling was reduced significantly for CsA compared to vehicle control when ear swelling was measured 24 hrs after challenge (i.e. 2 days after treatment) (peptide 100: 0.736±0.026 mm, p<0.0001; vehicle: 0.849±0.032 mm; CsA: 0.601±0.033 mm, p<0.0001) and when ear swelling was measured 48 hrs after challenge (i.e. 3 days after treatment) (peptide 100: 0.671±0.022 mm, p<0.0001; vehicle: 0.777±0.036 mm; CsA: 0.551±0.021 mm, p<0.0001).

Ear swelling was significantly reduced compared to vehicle control when rats were ear challenged on day 11 (i.e. 5 days after treatment) with doses of peptide 100 of 1, 3, 10, 30 or 100 nmol/kg when ear swelling was measured 24 hrs after challenge (i.e. 6 days after treatment) (peptide 100 at 1 nmol/kg: 0.872±0.037 mm, p=0.0872; 3 nmol/kg: 0.814±0.014 mm, p<0.0001; 10 nmol/kg: 0.796±0.043 mm, p<0.0001; 30 nmol/kg: 0.754±0.017 mm, p<0.0001; 100 nmol/kg: 0.751±0.009 mm, p<0.0001; vehicle: 0.902±0.028 mm) and when ear swelling was measured 48 hrs after challenge (i.e. 7 days after treatment) (peptide 100 at 1 nmol/kg: 0.854±0.038 mm, p=0.1953; 3 nmol/kg: 0.814±0.019 mm, p=0.0001; 10 nmol/kg: 0.794±0.036 mm, p=0.0001; 30 nmol/kg: 0.736±0.03 mm, p<0.0001; 100 nmol/kg: 0.731±0.016 mm, p<0.0001; vehicle: 0.876±0.027 mm).

In conclusion, a dose of 3 nmol/kg of peptide 100 is a minimal dose to obtain effect in the KLH-induced DTH model.

TABLE 15
Mean ear thickness (mm) ± SD of KLH-induced ear measured 24 hrs post-challenge

| | Groups | | | | | | | | |
|---|---|---|---|---|---|---|---|---|---|
| | 1 | 2 | 3 | 4 | 5 | 6 | 7 | 8 | 9 |
| | Treatment | | | | | | | | |
| | | Ptd 100 | | | Ptd 100 | Ptd 100 | Ptd 100 | Ptd 100 | Ptd 100 |
| | | 100 | | | 1 | 3 | 10 | 30 | 100 |
| | Veh. | nmol/kg | CsA | Veh. | nmol/kg | nmol/kg | nmol/kg | nmol/kg | nmol/kg |
| | Day measured (post-treatment) | | | | | | | | |
| | 2 | 2 | 2 | 6 | 6 | 6 | 6 | 6 | 6 |
| Mean | 0.849 | 0.736 | 0.601 | 0.902 | 0.872 | 0.814 | 0.796 | 0.754 | 0.751 |
| Std. Dev. | 0.032 | 0.026 | 0.033 | 0.028 | 0.037 | 0.014 | 0.043 | 0.017 | 0.009 |
| p-value | | p < 0.0001 | p < 0.0001 | | p = 0.0872 | p < 0.0001 | p < 0.0001 | p < 0.0001 | p < 0.0001 |

TABLE 16
Mean ear thickness (mm) ± SD of KLH-induced ear measured 48 hrs post-challenge

| | Groups | | | | | | | | |
|---|---|---|---|---|---|---|---|---|---|
| | 1 | 2 | 3 | 4 | 5 | 6 | 7 | 8 | 9 |
| | Treatment | | | | | | | | |
| | | Ptd 100 | | | Ptd 100 | Ptd 100 | Ptd 100 | Ptd 100 | Ptd 100 |
| | | 100 | | | 1 | 3 | 10 | 30 | 100 |
| | Veh. | nmol/kg | CsA | Veh. | nmol/kg | nmol/kg | nmol/kg | nmol/kg | nmol/kg |
| | Day measured (post-treatment) | | | | | | | | |
| | 3 | 3 | 3 | 7 | 7 | 7 | 7 | 7 | 7 |
| Mean | 0.777 | 0.671 | 0.551 | 0.876 | 0.854 | 0.814 | 0.794 | 0.736 | 0.731 |
| Std. Dev. | 0.036 | 0.022 | 0.021 | 0.027 | 0.038 | 0.019 | 0.036 | 0.03 | 0.016 |
| p-value | | p = 0.0008 | p < 0.0001 | | p = 0.0009 | p < 0.0001 | p < 0.0001 | p < 0.0001 | |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology or related fields are intended to be within the scope of the following aspects.

1. A Kv1.3 inhibitor, or a pharmaceutically acceptable salt thereof, for use in a method of treating or preventing a disease or disorder in a subject, wherein the Kv1.3 inhibitor comprises or consists of a peptide comprising or consisting of the sequence QMDMRCSASVECKQKCLKAIGSIFGKCMNKKCKCYPR (SEQ ID NO 1) or a variant thereof, wherein the variant
(a) has at least 70% sequence identity to SEQ ID NO 1, and/or
(b) differs from SEQ ID NO 1 by up to nine substitutions, insertions and/or deletions in total,
and wherein the method comprises administering the Kv1.3 inhibitor to the subject once every 2 to 8 days.

2. The Kv1.3 inhibitor or pharmaceutically acceptable salt for use according to claim 1, wherein the Kv1.3 inhibitor has an IC₅₀ for human Kv1.3 potassium channel of 400 nM or less, preferably 300 nM or less, preferably 50 nM or less, preferably 15 nM or less, preferably 10 nM or less, preferably 5 nM or less, preferably 2 nM or less.

3. The Kv1.3 inhibitor or pharmaceutically acceptable salt for use according to claim 1 or claim 2, wherein the variant has at least 70% sequence identity to SEQ ID NO 1, preferably at least 75%, at least 80%, at least 85%, at least 90%, at least 95%, at least 96%, at least 97%, at least 98%, at least 99%, or 100% sequence identity to SEQ ID NO 1.

4. The Kv1.3 inhibitor or pharmaceutically acceptable salt for use according to any one of claims 1 to 3, wherein the peptide comprises the following amino acids:
the amino acid at position 6 is C;
the amino acid at position 12 is C;
the amino acid at position 16 is C;
the amino acid at position 27 is C;
the amino acid at position 32 is C; and
the amino acid at position 34 is C;
preferably wherein also:
the amino acid at position 24 is F;
the amino acid at position 25 is G;
the amino acid at position 29 is N; and
the amino acid at position 31 is K.

5. The Kv1.3 inhibitor or pharmaceutically acceptable salt for use according to any one of claims 1 to 4, wherein the variant differs from SEQ ID NO 1 by up to nine substitutions, insertions and/or deletions in total, preferably up to eight, up to seven, up to six, up to five, up to four, up to three or up to two substitutions, insertions and/or deletions in total or by one substitution, insertion or deletion in total.

6. The Kv1.3 inhibitor or pharmaceutically acceptable salt for use according to any one of claims 1 to 5, wherein any substitutions or deletions in the variant of SEQ ID NO 1 are at amino acid positions selected from positions 1-5, 7-11, 13-15, 17-23, 26, 28, 30, 33 and 35-37 of SEQ ID NO 1, preferably at amino acid positions selected from positions 1, 2, 3, 4, 5, 7, 14, 18, 19, 28 and 37 of SEQ ID NO 1.

7. The Kv1.3 inhibitor or pharmaceutically acceptable salt for use according to any one of claims 1 to 6, wherein the peptide comprises the following amino acids:
the amino acid at position 1 is H, N, P, p, Q, S, V or Y or is deleted;
the amino acid at position 2 is I, M or Nle or is deleted;
the amino acid at position 3 is D, E or S or is deleted;
the amino acid at position 4 is E, L, M, Nle, S or V or is deleted;
the amino acid at position 5 is R or K or is deleted;
the amino acid at position 7 is E, F, H, K, Orn, R, S, Y, 2,3-Diaminopropanoyl, 2,4-Diaminobutanoyl or 2-Amino-3-guanidinopropionyl;
the amino acid at position 8 is A, H, I, L, S or Y;
the amino acid at position 9 is F, L, P, S, Orn, V, Abu or 2,3-Diaminopropanoyl;
the amino acid at position 10 is K, P, Q, R or V;
the amino acid at position 11 is E or Q;
the amino acid at position 13 is A, E, G, K, L, Q or V;
the amino acid at position 14 is E, K, L, Q, V or 2-amino-5-carboxypentanoyl;
the amino acid at position 15 is K, L, P, S;
the amino acid at position 17 is K, L, R or Y or is deleted;
the amino acid at position 18 is A, D, G, K, Q, hQ, V or Y or is deleted;
the amino acid at position 19 is A, K, R or Y or is deleted;
the amino acid at position 20 is E, I, R or Y or is deleted;
the amino acid at position 21 is E, G, H or R;
the amino acid at position 22 is C, R or S;
the amino acid at position 23 is G, I, K, P or R;
the amino acid at position 26 is K or hK;
the amino acid at position 28 is M or Nle;
the amino acid at position 30 is G or K;
the amino acid at position 33 is H, K, R or V;
the amino acid at position 35 is Y, F(4-F), F(4-CH₃), F(4-NO₂) or F(4-NH₂);
the amino acid at position 36 is Q or P or is deleted; and/or
the amino acid at position 37 is C, G, R, S or (4-amino-5-hydroxypentyl)guanidine or is deleted.

8. The Kv1.3 inhibitor or pharmaceutically acceptable salt for use according to any one of claims 1 to 7, wherein the peptide comprises or consists of one of the following sequences:

| SEQ | |
|---|---|
| ID NO | Sequence |
| 1 | QMDMRCSASVECKQKCLKAIGSIFGKCMNKKCKCYPR |
| 2 | QMDMRCSASVECKQKCLKAIGRGFGKCMNKKCKCYPR |
| 3 | NMDMRCSASVECKQKCLKAIGSIFGKCMNKKCKCYPR |
| 4 | NMDMRCSASVECKQKCLKAIGRGFGKCMNKKCKCYPR |
| 5 | MDMRCSASVECKQKCLKAIGSIFGKCMNKKCKCYPR |
| 6 | DMRCSASVECKQKCLKAIGSIFGKCMNKKCKCYPR |
| 7 | P[Nle]E[Nle]RCYASVECKQKCLAAIGSIFGKC[Nle]NKKCKCYPR |
| 8 | NIDMRCSASVECKQKCLKAIGSIFGKCMNKKCKCYPR |
| 9 | NMDVRCSASVECKQKCLKAIGSIFGKCMNKKCKCYPR |
| 10 | NMDMRCSASVECKQKCKDAIGSIFGKCMNKKCKCYPR |
| 11 | GGNMDMRCSASVECKQKCLKAIGSIFGKCMNKKCKCYPR |
| 12 | NMEMRCSASVECKQKCLKAIGSIFGKCMNKKCKCYPR |
| 13 | SGNMDMRCSASVECKQKCLKAIGSIFGKCMNKKCKCYPR |
| 14 | N[Nle]DMRCSASVECKQKCLKAIGSIFGKCMNKKCKCYPR |
| 15 | NMD[Nle]RCSASVECKQKCLKAIGSIFGKCMNKKCKCYPR |
| 16 | NMDMRCSASVECKQKCLKAIGSIFGKC[Nle]NKKCKCYPR |
| 17 | NMDMRCSASVECKVKCLKAIGSIFGKCMNKKCKCYPR |
| 18 | NMDMRCSASVECKQLCLKAIGSIFGKCMNKKCKCYPR |
| 19 | NMDMRCSASVECKQKCKKAIGSIFGKCMNKKCKCYPR |
| 20 | NMDMRCSASVECKQKCLDAIGSIFGKCMNKKCKCYPR |
| 21 | NMDMRCSASVECKQKCLKAIRSIFGKCMNKKCKCYPR |
| 22 | NMDMRCSASVECKQKCLKAIESIFGKCMNKKCKCYPR |
| 23 | NMDMRCSASVECKQKCLKAIGSIFGKCMNKKCKCYPRRRTA |
| 24 | NMDMRCSASVECKQKCLKAIGSIFGKCMNKKCKCYPRHRRK |
| 25 | NMDMRCKASVECKQKCLKAIGSIFGKCMNKKCKCYPR |
| 26 | NMDMRCSISVECKQKCLKAIGSIFGKCMNKKCKCYPR |
| 27 | NMDMRCSASRECKQKCLKAIGSIFGKCMNKKCKCYPR |
| 28 | NMDMRCSASVQCKQKCLKAIGSIFGKCMNKKCKCYPR |
| 29 | NMDMRCSASVECLQKCLKAIGSIFGKCMNKKCKCYPR |
| 30 | NMDMRCSASVECAQKCLKAIGSIFGKCMNKKCKCYPR |
| 31 | NMDMRCSASVECKEKCLKAIGSIFGKCMNKKCKCYPR |
| 32 | NMDMRCSASVECKLKCLKAIGSIFGKCMNKKCKCYPR |
| 33 | NMDMRCSASVECKQKCLKAIHSIFGKCMNKKCKCYPR |
| 34 | NMDMRCSASVECKQKCLKAIGSKFGKCMNKKCKCYPR |
| 35 | NMDMRCSASVECKQKCLKAIGSRFGKCMNKKCKCYPR |
| 36 | NMDMRCSASVECKQKCLKAIGSIFGKCMNGKCKCYPR |
| 37 | NMDMRCSASVECKQKCLKAIGSIFGKCMNKKCHCYPR |
| 38 | NMDMRCSASVECKQKCLKAIGSIFGKCMNKKCVCYPR |
| 39 | N[Nle]D[Nle]RCSASVECKQKCLKAIGSIFGKCMNKKCKCYPR |
| 40 | N[Nle]D[Nle]RCSASVECKQKCLKAIGSIFGKC[Nle]NKKCKCYPR |
| 41 | P[Nle]D[Nle]RCSASVECKQKCLKAIGSIFGKC[Nle]NKKCKCYPR |
| 42 | N[Nle]D[Nle]RCRASVECKQKCLKAIGSIFGKC[Nle]NKKCKCYPR |
| 43 | N[Nle]D[Nle]RCSASVECEQKCLKAIGSIFGKC[Nle]NKKCKCYPR |
| 44 | N[Nle]D[Nle]RCSASVECQQKCLKAIGSIFGKC[Nle]NKKCKCYPR |
| 45 | N[Nle]D[Nle]RCSASVECKKKCLKAIGSIFGKC[Nle]NKKCKCYPR |
| 46 | N[Nle]S[Nle]RCSASVECKQKCLKAIGSIFGKC[Nle]NKKCKCYPR |
| 47 | N[Nle]D[Nle]RCSHSVECKQKCLKAIGSIFGKC[Nle]NKKCKCYPR |
| 48 | N[Nle]D[Nle]RCSASVECKQSCLKAIGSIFGKC[Nle]NKKCKCYPR |
| 49 | N[Nle]D[Nle]RCSASVECKQKCKAIGSIFGKC[Nle]NKKCKCYPR |
| 50 | NMDMRCSASVECKQKCYKAIGSIFGKCMNKKCKCYPR |
| 51 | NMDMRCSASVECKQKCRKAIGSIFGKCMNKKCKCYPR |
| 52 | NMDMRCSASVECKQKCLAAIGSIFGKCMNKKCKCYPR |
| 53 | NMDMRCSASVECKQKCLYAIGSIFGKCMNKKCKCYPR |
| 54 | NMDMRCSASVECKQKCLAIGSIFGKCMNKKCKCYPR |
| 55 | NMDMRCSASVECKQKCLKYIGSIFGKCMNKKCKCYPR |
| 56 | NMDMRCSASVECKQKCLKRIGSIFGKCMNKKCKCYPR |
| 57 | NMDMRCSASVECKQKCLKIGSIFGKCMNKKCKCYPR |
| 58 | NMDMRCSASVECKQKCLKAYGSIFGKCMNKKCKCYPR |
| 59 | NMDMRCSASVECKQKCLKAEGSIFGKCMNKKCKCYPR |
| 60 | NMDMRCSASVECKQKCLKARGSIFGKCMNKKCKCYPR |
| 61 | NMDMRCSASVECKQKCLKAGSIFGKCMNKKCKCYPR |
| 62 | N[Nle]D[Nle]RCSASVECKQKCLKAIGSPFGKC[Nle]NKKCKCYPR |
| 63 | N[Nle]D[Nle]RCSASKECKQKCLKAIGSIFGKC[Nle]NKKCKCYPR |
| 64 | H[Nle]D[Nle]RCSASVECKQKCLKAIGSIFGKC[Nle]NKKCKCYPR |
| 65 | Y[Nle]D[Nle]RCSASVECKQKCLKAIGSIFGKC[Nle]NKKCKCYPR |
| 66 | S[Nle]D[Nle]RCSASVECKQKCLKAIGSIFGKC[Nle]NKKCKCYPR |
| 67 | V[Nle]D[Nle]RCSASVECKQKCLKAIGSIFGKC[Nle]NKKCKCYPR |
| 68 | S[Nle]D[Nle]RCSA[Abu]VECKQKCLKAIGSIFGKCMNKKCKCYPR |
| 69 | S[Nle]D[Nle]RCSASVECKQKCLKAIGSIFG[homo-Lys]CMNKKCKCYPR |
| 70 | S[Nle]D[Nle]RCSASVECKQKCLKAIGSIFGKCMNKKCKC[F(4-NH2)]PR |
| 71 | S[Nle]D[Nle]RCSASVECGQKCLKAIGSIFGKCMNKKCKCYPR |
| 72 | S[Nle]D[Nle]RCSASVECVQKCLKAIGSIFGKCMNKKCKCYPR |
| 73 | S[Nle]D[Nle]RCSASVECK[2-Amino-5- |
| | carboxypentanoyl]KCLKAIGSIFGKCMNKKCKCYPR |
| 74 | S[Nle]D[Nle]RCSASVECKQKCLKAIGSIFGKCMNKKCKCYQ |
| 75 | S[Nle]D[Nle]RCSASVECKQKCLKAIGSIFGKCMNKKCRCYPR |
| 76 | S[Nle]DERCSASVECKQKCLKAIGSIFGKCMNKKCKCYPR |
| 77 | S[Nle]D[Nle]RCSASVECKQKCLGAIGSIFGKCMNKKCKCYPR |
| 78 | S[Nle]D[Nle]RCSASVECKQKCLVAIGSIFGKCMNKKCKCYPR |
| 79 | S[Nle]D[Nle]RCSASVECAQSCLKAIGSIFGKCMNKKCKCYPR |
| 80 | S[Nle]D[Nle]RCSASVECAQKCLAAIGSIFGKCMNKKCKCYPR |
| 81 | S[Nle]D[Nle]RCSASVECAQLCLAAIGSIFGKCMNKKCKCYPR |
| 82 | P[Nle]E[Nle]RC[2,4- |
| | Diaminobutanoyl]ASVECKQKCLAAIGSIFGKC[Nle]NKKCKCYPR |
| 83 | P[Nle]D[Nle]RCSASVECKQKCLKAIGCIFGKC[Nle]NKKCKCYPC |
| 84 | S[Nle]D[Nle]RCSASVECKEKCLQAIGSIFGKC[Nle]NKKCKCYPR |
| 85 | P[Nle]D[Nle]RCSASVECKEKCL[homo-Gln]AIGSIFGKC[Nle]NKKCKCYPR |
| 86 | CSASVECKQKCLKAIGCIFGKC[Nle]NKKCKCYPC |
| 87 | S[Nle]D[Nle]RCSASVECKQKCLAAIGCIFGKC[Nle]NKKCKCYPC |
| 88 | P[Nle]E[Nle]RC[2-Amino-3- |
| | guanidinopropionyl]ASVECKQKCLAAIGSIFGKC[Nle]NKKCKCYPR |
| 89 | S[Nle]D[Nle]RCSALVECKQKCLKAIGSIFGKCMNKKCKCYPR |
| 90 | S[Nle]D[Nle]RCSAVVECKQKCLKAIGSIFGKCMNKKCKCYPR |
| 91 | S[Nle]D[Nle]RCSASVECKQKCLKAIGSIFGKCMNKKCKC[F(4-F)]PR |
| 92 | S[Nle]D[Nle]RCSASVECKQKCLKAIGSIFGKCMNKKCKC[F(4-NO₂)]PR |
| 93 | S[Nle]D[Nle]RCSASVECKQKCLKAIGSIFGKCMNKKCKC[F(4-CH₃)]PR |
| 94 | [Nle]D[Nle]RCSASVECKQKCLKAIGSIFGKC[Nle]NKKCKCYPR |
| 95 | NID[Nle]RCSASVECKQKCLKAIGSIFGKC[Nle]NKKCKCYPR |
| 96 | PIE[Nle]RCSASVECKQKCLKAIGSIFGKC[Nle]NKKCKCYPR |
| 97 | P[Nle]E[Nle]RCSASVECKQKCLAAIGSIFGKC[Nle]NKKCKCYPR |
| 98 | P[Nle]E[Nle]RCSA[Orn]VECKQKCLAAIGSIFGKC[Nle]NKKCKCYPR |
| 99 | P[Nle]E[Nle]RCSASVECKQKCLLAIGSIFGKC[Nle]NKKCKCYPR |
| 100 | PIDERCSASVECKQKCLAAIGSIFGKC[Nle]NKKCKCYPR |
| 101 | PIE[Nle]RCSASVECKQKCLAAIGSIFGKC[Nle]NKKCKCYPR |
| 102 | P[Nle]D[Nle]RCSASVECAQKCLAAIGSIFGKCMNKKCKCYPR |
| 103 | P[Nle]E[Nle]RCSASVECAQKCLAAIGSIFGKC[Nle]NKKCKCYPR |
| 104 | CSASVECKQKCLKAIGSIFGKC[Nle]NKKCKCYPR |
| 105 | P[Nle]E[Nle]RC[2,3- |
| | Diaminopropanoyl]ASVECKQKCLAAIGSIFGKC[Nle]NKKCKCYPR |
| 106 | RCSASVECKQKCLKAIGSIFGKC[Nle]NKKCKCYPR |
| 107 | SKCSASVECKQKCLKAIGSIFGKC[Nle]NKKCKCYPR |
| 108 | LRCSASVECKQKCLKAIGSIFGKC[Nle]NKKCKCYPR |
| 109 | CSASVECKQKCLKAIGSIFGKC[Nle]NKKCKCYPS |
| 110 | CSASVECKQKCLKAIGSIFGKC[Nle]NKKCKCYS |
| 111 | CSASVECKQKCLKAIGSIFGKC[Nle]NKKCKCYG |
| 112 | CSASVECKQKCLKAIGSIFGKC[Nle]NKKCKCY |
| 113 | P[Nle]E[Nle]RC[Orn]ASVECKQKCLAAIGSIFGKC[Nle]NKKCKCYPR |
| 114 | P[Nle]E[Nle]RCHASVECKQKCLAAIGSIFGKC[Nle]NKKCKCYPR |
| 115 | LRCSASVECKQKCLAAIGSIFGKC[Nle]NKKCKCYPR |
| 116 | LRCSASVECKQKCLAAIGSIFGKCMNKKCKCYPR |
| 117 | CSASVECKQKCLKAIGSIFGKCMNKKCKCYPR |
| 118 | P[Nle]E[Nle]RCSASVECKEKCLAAIGSIFGKC[Nle]NKKCKCYPR |
| 119 | p[Nle]E[Nle]RCSASVECKQKCLAAIGSIFGKC[Nle]NKKCKCYPR |
| 120 | P[Nle]E[Nle]RCSASVECKQKCLAAIGSIFGKC[Nle]NKKCKCYR |
| 121 | [Nle]E[Nle]RCSASVECKQKCLAAIGSIFGKC[Nle]NKKCKCYPR |
| 122 | P[Nle]E[Nle]RCSASVECKQKCLAAIGSIFGKC[Nle]NKKCKCYPRR |
| 123 | P[Nle]E[Nle]RCSASVECKQKCLAAIGSIFGKC[Nle]NKKCKCYPRY |
| 124 | P[Nle]E[Nle]RCSASVECKQKCLAAIGSIFGKC[Nle]NKKCKCYPRL |
| 125 | P[Nle]E[Nle]RCSASVECKQKCLAAIGSIFGKC[Nle]NKKCKCYPRH |
| 126 | P[Nle]E[Nle]RCSASVECKQKCLAAIGSIFGKC[Nle]NKKCKCYPRE |
| 127 | P[Nle]E[Nle]RCSASVECKQKCLAAIGSIFGKC[Nle]NKKCKCYPRKS |
| 128 | P[Nle]E[Nle]RCSASVECKQKCLAAIGSIFGKC[Nle]NKKCKCYPRFE |
| 129 | P[Nle]E[Nle]RCSASVECKQKCLAAIGSIFGKC[Nle]NKKCKCYPRHR |
| 130 | P[Nle]E[Nle]RCSASVECKQKCLAAIGSIFGKC[Nle]NKKCKCYPRAK |
| 131 | P[Nle]E[Nle]RCSASVECKQKCLAAIGSIFGKC[Nle]NKKCKCYP-[(4-amino-5- |
| | hydroxypentyl)guanidine] |
| 132 | P[Nle]E[Nle]RCSASVECKQKCLAAIGSIFGKC[Nle]NKKCKCYPR-[4-amino-5- |
| | hydroxypentanamide] |
| 133 | P[Nle]E[Nle]RCSASVECKQKCLAAIGSIFGKC[Nle]NKKCKCYPRST |
| 134 | P[Nle]E[Nle]RCSASVECKQKCLAAIGSIFGKC[Nle]NKKCKCYPRRY |
| 135 | P[Nle]E[Nle]RCSSSVECKQKCLAAIGSIFGKC[Nle]NKKCKCYPR |
| 136 | P[Nle]E[Nle]RCSLSVECKQKCLAAIGSIFGKC[Nle]NKKCKCYPR |
| 137 | P[Nle]E[Nle]RCSAPVECKQKCLAAIGSIFGKC[Nle]NKKCKCYPR |
| 138 | P[Nle]E[Nle]RCSASPECKQKCLAAIGSIFGKC[Nle]NKKCKCYPR |
| 139 | P[Nle]E[Nle]RCSASQECKQKCLAAIGSIFGKC[Nle]NKKCKCYPR |
| 140 | P[Nle]E[Nle]RCSASVECLQKCLAAIGSIFGKC[Nle]NKKCKCYPR |
| 141 | P[Nle]E[Nle]RCSASVECKQPCLAAIGSIFGKC[Nle]NKKCKCYPR |
| 142 | P[Nle]E[Nle]RCEASVECKQKCLAAIGSIFGKC[Nle]NKKCKCYPR |
| 143 | P[Nle]E[Nle]RCFASVECKQKCLAAIGSIFGKC[Nle]NKKCKCYPR |
| 144 | P[Nle]E[Nle]RCSYSVECKQKCLAAIGSIFGKC[Nle]NKKCKCYPR |
| 145 | P[Nle]E[Nle]RCSAFVECKQKCLAAIGSIFGKC[Nle]NKKCKCYPR |
| 146 | P[Nle]E[Nle]RCSA[2,3- |
| | Diaminopropanoyl]VECKQKCLAAIGSIFGKC[Nle]NKKCKCYPR |
| 147 | P[Nle]E[Nle]RC[2,4- |
| | Diaminobutanoyl]ASVECAQSCLAAIGSIFGKC[Nle]NGKCKCYPR |
| 148 | P[Nle]E[Nle]RCRASVECAQSCLAAIGSIFGKC[Nle]NGKCKCYPR |
| 149 | P[Nle]E[Nle]RCSASRECAQSCLAAIGSIFGKC[Nle]NGKCKCYPR |
| 150 | P[Nle]E[Nle]RCSASVECKQKCLAKIGSIFGKC[Nle]NKKCKCYPR |

9. The Kv1.3 inhibitor or pharmaceutically acceptable salt for use according to any one of claims 1 to 8, wherein the peptide is selected from the following peptides:

| SEQ | | |
|---|---|---|
| ID | Ptd | |
| NO | No. | Peptide |
| 1 | 1 | H-QMDMRC(1)SASVEC(2)KQKC(3)LKAIGSIFGKC(1)MNKKC(2)KC(3)YPR-OH |
| 2 | 2 | H-QMDMRC(1)SASVEC(2)KQKC(3)LKAIGRGFGKC(1)MNKKC(2)KC(3)YPR-OH |
| 3 | 3 | H-NMDMRC(1)SASVEC(2)KQKC(3)LKAIGSIFGKC(1)MNKKC(2)KC(3)YPR-OH |
| 3 | 4 | H-NMDMRC(1)SASVEC(2)KQKC(3)LKAIGSIFGKC(1)MNKKC(2)KC(3)YPR-NH₂ |
| 4 | 5 | H-NMDMRC(1)SASVEC(2)KQKC(3)LKAIGRGFGKC(1)MNKKC(2)KC(3)YPR-OH |
| 5 | 6 | H-MDMRC(1)SASVEC(2)KQKC(3)LKAIGSIFGKC(1)MNKKC(2)KC(3)YPR-OH |
| 6 | 7 | H-DMRC(1)SASVEC(2)KQKC(3)LKAIGSIFGKC(1)MNKKC(2)KC(3)YPR-OH |
| 7 | 8 | H- |
| | | P[Nle]E[Nle]RC(1)YASVEC(2)KQKC(3)LAAIGSIFGKC(1)[Nle]NKKC(2)KC(3)YPR- |
| | | OH |
| 8 | 9 | H-NIDMRC(1)SASVEC(2)KQKC(3)LKAIGSIFGKC(1)MNKKC(2)KC(3)YPR-OH |
| 9 | 10 | H-NMDVRC(1)SASVEC(2)KQKC(3)LKAIGSIFGKC(1)MNKKC(2)KC(3)YPR-OH |
| 10 | 11 | H-NMDMRC(1)SASVEC(2)KQKC(3)KDAIGSIFGKC(1)MNKKC(2)KC(3)YPR-OH |
| 11 | 12 | H-GGNMDMRC(1)SASVEC(2)KQKC(3)LKAIGSIFGKC(1)MNKKC(2)KC(3)YPR-OH |
| 12 | 13 | H-NMEMRC(1)SASVEC(2)KQKC(3)LKAIGSIFGKC(1)MNKKC(2)KC(3)YPR-OH |
| 13 | 14 | H-SGNMDMRC(1)SASVEC(2)KQKC(3)LKAIGSIFGKC(1)MNKKC(2)KC(3)YPR-OH |
| 14 | 15 | H-N[Nle]DMRC(1)SASVEC(2)KQKC(3)LKAIGSIFGKC(1)MNKKC(2)KC(3)YPR-NH₂ |
| 15 | 16 | H-NMD[Nle]RC(1)SASVEC(2)KQKC(3)LKAIGSIFGKC(1)MNKKC(2)KC(3)YPR-NH₂ |
| 16 | 17 | H-NMDMRC(1)SASVEC(2)KQKC(3)LKAIGSIFGKC(1)[Nle]NKKC(2)KC(3)YPR-NH₂ |
| 17 | 18 | H-NMDMRC(1)SASVEC(2)KVKC(3)LKAIGSIFGKC(1)MNKKC(2)KC(3)YPR-NH₂ |
| 18 | 19 | H-NMDMRC(1)SASVEC(2)KQLC(3)LKAIGSIFGKC(1)MNKKC(2)KC(3)YPR-NH₂ |
| 19 | 20 | H-NMDMRC(1)SASVEC(2)KQKC(3)KKAIGSIFGKC(1)MNKKC(2)KC(3)YPR-NH₂ |
| 20 | 21 | H-NMDMRC(1)SASVEC(2)KQKC(3)LDAIGSIFGKC(1)MNKKC(2)KC(3)YPR-NH₂ |
| 21 | 22 | H-NMDMRC(1)SASVEC(2)KQKC(3)LKAIRSIFGKC(1)MNKKC(2)KC(3)YPR-NH₂ |
| 22 | 23 | H-NMDMRC(1)SASVEC(2)KQKC(3)LKAIESIFGKC(1)MNKKC(2)KC(3)YPR-NH₂ |
| 23 | 24 | H-NMDMRC(1)SASVEC(2)KQKC(3)LKAIGSIFGKC(1)MNKKC(2)KC(3)YPRRRTA- |
| | | NH₂ |
| 24 | 25 | H-NMDMRC(1)SASVEC(2)KQKC(3)LKAIGSIFGKC(1)MNKKC(2)KC(3)YPRHRRK- |
| | | NH₂ |
| 25 | 26 | H-NMDMRC(1)KASVEC(2)KQKC(3)LKAIGSIFGKC(1)MNKKC(2)KC(3)YPR-NH₂ |
| 26 | 27 | H-NMDMRC(1)SISVEC(2)KQKC(3)LKAIGSIFGKC(1)MNKKC(2)KC(3)YPR-NH₂ |
| 27 | 28 | H-NMDMRC(1)SASREC(2)KQKC(3)LKAIGSIFGKC(1)MNKKC(2)KC(3)YPR-NH₂ |
| 28 | 29 | H-NMDMRC(1)SASVQC(2)KQKC(3)LKAIGSIFGKC(1)MNKKC(2)KC(3)YPR-NH₂ |
| 29 | 30 | H-NMDMRC(1)SASVEC(2)LQKC(3)LKAIGSIFGKC(1)MNKKC(2)KC(3)YPR-NH₂ |
| 30 | 31 | H-NMDMRC(1)SASVEC(2)AQKC(3)LKAIGSIFGKC(1)MNKKC(2)KC(3)YPR-NH₂ |
| 31 | 32 | H-NMDMRC(1)SASVEC(2)KEKC(3)LKAIGSIFGKC(1)MNKKC(2)KC(3)YPR-NH₂ |
| 32 | 33 | H-NMDMRC(1)SASVEC(2)KLKC(3)LKAIGSIFGKC(1)MNKKC(2)KC(3)YPR-NH₂ |
| 33 | 34 | H-NMDMRC(1)SASVEC(2)KQKC(3)LKAIHSIFGKC(1)MNKKC(2)KC(3)YPR-NH₂ |
| 34 | 35 | H-NMDMRC(1)SASVEC(2)KQKC(3)LKAIGSKFGKC(1)MNKKC(2)KC(3)YPR-NH₂ |
| 35 | 36 | H-NMDMRC(1)SASVEC(2)KQKC(3)LKAIGSRFGKC(1)MNKKC(2)KC(3)YPR-NH₂ |
| 36 | 37 | H-NMDMRC(1)SASVEC(2)KQKC(3)LKAIGSIFGKC(1)MNGKC(2)KC(3)YPR-NH₂ |
| 37 | 38 | H-NMDMRC(1)SASVEC(2)KQKC(3)LKAIGSIFGKC(1)MNKKC(2)HC(3)YPR-NH₂ |
| 38 | 39 | H-NMDMRC(1)SASVEC(2)KQKC(3)LKAIGSIFGKC(1)MNKKC(2)VC(3)YPR-NH₂ |
| 39 | 40 | H-N[Nle]D[Nle]RC(1)SASVEC(2)KQKC(3)LKAIGSIFGKC(1)MNKKC(2)KC(3)YPR- |
| | | NH₂ |
| 40 | 41 | H- |
| | | N[Nle]D[Nle]RC(1)SASVEC(2)KQKC(3)LKAIGSIFGKC(1)[Nle]NKKC(2)KC(3)YPR- |
| | | NH₂ |
| 41 | 42 | H- |
| | | P[Nle]D[Nle]RC(1)SASVEC(2)KQKC(3)LKAIGSIFGKC(1)[Nle]NKKC(2)KC(3)YPR- |
| | | NH₂ |
| 42 | 43 | H- |
| | | N[Nle]D[Nle]RC(1)RASVEC(2)KQKC(3)LKAIGSIFGKC(1)[Nle]NKKC(2)KC(3)YPR- |
| | | NH₂ |
| 43 | 44 | H- |
| | | N[Nle]D[Nle]RC(1)SASVEC(2)EQKC(3)LKAIGSIFGKC(1)[Nle]NKKC(2)KC(3)YPR- |
| | | NH₂ |
| 44 | 45 | H- |
| | | N[Nle]D[Nle]RC(1)SASVEC(2)QQKC(3)LKAIGSIFGKC(1)[Nle]NKKC(2)KC(3)YPR- |
| | | NH₂ |
| 45 | 46 | H- |
| | | N[Nle]D[Nle]RC(1)SASVEC(2)KKKC(3)LKAIGSIFGKC(1)[Nle]NKKC(2)KC(3)YPR- |
| | | NH₂ |
| 46 | 47 | H- |
| | | N[Nle]S[Nle]RC(1)SASVEC(2)KQKC(3)LKAIGSIFGKC(1)[Nle]NKKC(2)KC(3)YPR- |
| | | NH₂ |
| 47 | 48 | H- |
| | | N[Nle]D[Nle]RC(1)SHSVEC(2)KQKC(3)LKAIGSIFGKC(1)[Nle]NKKC(2)KC(3)YPR- |
| | | NH₂ |
| 48 | 49 | H- |
| | | N[Nle]D[Nle]RC(1)SASVEC(2)KQSC(3)LKAIGSIFGKC(1)[Nle]NKKC(2)KC(3)YPR- |
| | | NH₂ |
| 49 | 50 | H-N[Nle]D[Nle]RC(1)SASVEC(2)KQKC(3)KAIGSIFGKC(1)[Nle]NKKC(2)KC(3)YPR- |
| | | NH₂ |
| 50 | 51 | H-NMDMRC(1)SASVEC(2)KQKC(3)YKAIGSIFGKC(1)MNKKC(2)KC(3)YPR-NH₂ |
| 51 | 52 | H-NMDMRC(1)SASVEC(2)KQKC(3)RKAIGSIFGKC(1)MNKKC(2)KC(3)YPR-NH₂ |
| 52 | 53 | H-NMDMRC(1)SASVEC(2)KQKC(3)LAAIGSIFGKC(1)MNKKC(2)KC(3)YPR-NH₂ |
| 53 | 54 | H-NMDMRC(1)SASVEC(2)KQKC(3)LYAIGSIFGKC(1)MNKKC(2)KC(3)YPR-NH₂ |
| 54 | 55 | H-NMDMRC(1)SASVEC(2)KQKC(3)LAIGSIFGKC(1)MNKKC(2)KC(3)YPR-NH₂ |
| 55 | 56 | H-NMDMRC(1)SASVEC(2)KQKC(3)LKYIGSIFGKC(1)MNKKC(2)KC(3)YPR-NH₂ |
| 56 | 57 | H-NMDMRC(1)SASVEC(2)KQKC(3)LKRIGSIFGKC(1)MNKKC(2)KC(3)YPR-NH₂ |
| 57 | 58 | H-NMDMRC(1)SASVEC(2)KQKC(3)LKIGSIFGKC(1)MNKKC(2)KC(3)YPR-NH₂ |
| 58 | 59 | H-NMDMRC(1)SASVEC(2)KQKC(3)LKAYGSIFGKC(1)MNKKC(2)KC(3)YPR-NH₂ |
| 59 | 60 | H-NMDMRC(1)SASVEC(2)KQKC(3)LKAEGSIFGKC(1)MNKKC(2)KC(3)YPR-NH₂ |
| 60 | 61 | H-NMDMRC(1)SASVEC(2)KQKC(3)LKARGSIFGKC(1)MNKKC(2)KC(3)YPR-NH₂ |
| 61 | 62 | H-NMDMRC(1)SASVEC(2)KQKC(3)LKAGSIFGKC(1)MNKKC(2)KC(3)YPR-NH₂ |
| 62 | 63 | H- |
| | | N[Nle]D[Nle]RC(1)SASVEC(2)KQKC(3)LKAIGSPFGKC(1)[Nle]NKKC(2)KC(3)YPR- |
| | | NH₂ |
| 63 | 64 | H- |
| | | N[Nle]D[Nle]RC(1)SASKEC(2)KQKC(3)LKAIGSIFGKC(1)[Nle]NKKC(2)KC(3)YPR- |
| | | NH₂ |
| 64 | 65 | H- |
| | | H[Nle]D[Nle]RC(1)SASVEC(2)KQKC(3)LKAIGSIFGKC(1)[Nle]NKKC(2)KC(3)YPR- |
| | | NH₂ |
| 65 | 66 | H- |
| | | Y[Nle]D[Nle]RC(1)SASVEC(2)KQKC(3)LKAIGSIFGKC(1)[Nle]NKKC(2)KC(3)YPR- |
| | | NH₂ |
| 66 | 67 | H- |
| | | S[Nle]D[Nle]RC(1)SASVEC(2)KQKC(3)LKAIGSIFGKC(1)[Nle]NKKC(2)KC(3)YPR- |
| | | NH₂ |
| 66 | 68 | H- |
| | | S[Nle]D[Nle]RC(1)SASVEC(2)KQKC(3)LKAIGSIFGKC(1)[Nle]NKKC(2)KC(3)YPR- |
| | | OH |
| 67 | 69 | H- |
| | | V[Nle]D[Nle]RC(1)SASVEC(2)KQKC(3)LKAIGSIFGKC(1)[Nle]NKKC(2)KC(3)YPR- |
| | | NH₂ |
| 68 | 70 | H- |
| | | S[Nle]D[Nle]RC(1)SA[Abu]VEC(2)KQKC(3)LKAIGSIFGKC(1)MNKKC(2)KC(3)YPR- |
| | | NH₂ |
| 69 | 71 | H-S[Nle]D[Nle]RC(1)SASVEC(2)KQKC(3)LKAIGSIFG[homo- |
| | | Lys]C(1)MNKKC(2)KC(3)YPR-NH₂ |
| 70 | 72 | H-S[Nle]D[Nle]RC(1)SASVEC(2)KQKC(3)LKAIGSIFGKC(1)MNKKC(2)KC(3)[F(4- |
| | | NH₂)]PR-NH₂ |
| 71 | 73 | H-S[Nle]D[Nle]RC(1)SASVEC(2)GQKC(3)LKAIGSIFGKC(1)MNKKC(2)KC(3)YPR- |
| | | NH₂ |
| 72 | 74 | H-S[Nle]D[Nle]RC(1)SASVEC(2)VQKC(3)LKAIGSIFGKC(1)MNKKC(2)KC(3)YPR- |
| | | NH₂ |
| 73 | 75 | H-S[Nle]D[Nle]RC(1)SASVEC(2)K[2-Amino-5- |
| | | carboxypentanoyl]KC(3)LKAIGSIFGKC(1)MNKKC(2)KC(3)YPR-NH₂ |
| 74 | 76 | H-S[Nle]D[Nle]RC(1)SASVEC(2)KQKC(3)LKAIGSIFGKC(1)MNKKC(2)KC(3)YQ- |
| | | NH₂ |
| 75 | 77 | H-S[Nle]D[Nle]RC(1)SASVEC(2)KQKC(3)LKAIGSIFGKC(1)MNKKC(2)RC(3)YPR- |
| | | NH₂ |
| 76 | 78 | H-S[Nle]DERC(1)SASVEC(2)KQKC(3)LKAIGSIFGKC(1)MNKKC(2)KC(3)YPR-NH₂ |
| 77 | 79 | H-S[Nle]D[Nle]RC(1)SASVEC(2)KQKC(3)LGAIGSIFGKC(1)MNKKC(2)KC(3)YPR- |
| | | NH₂ |
| 78 | 80 | H-S[Nle]D[Nle]RC(1)SASVEC(2)KQKC(3)LVAIGSIFGKC(1)MNKKC(2)KC(3)YPR- |
| | | NH₂ |
| 79 | 81 | H-S[Nle]D[Nle]RC(1)SASVEC(2)AQSC(3)LKAIGSIFGKC(1)MNKKC(2)KC(3)YPR- |
| | | NH₂ |
| 80 | 82 | H-S[Nle]D[Nle]RC(1)SASVEC(2)AQKC(3)LAAIGSIFGKC(1)MNKKC(2)KC(3)YPR- |
| | | NH₂ |
| 81 | 83 | H-S[Nle]D[Nle]RC(1)SASVEC(2)AQLC(3)LAAIGSIFGKC(1)MNKKC(2)KC(3)YPR- |
| | | NH₂ |
| 82 | 84 | H-P[Nle]E[Nle]RC(1)[2,4- |
| | | Diaminobutanoyl]ASVEC(2)KQKC(3)LAAIGSIFGKC(1)[Nle]NKKC(2)KC(3)YPR-OH |
| 83 | 85 | H- |
| | | P[Nle]D[Nle]RC(1)SASVEC(2)KQKC(3)LKAIGC(4)IFGKC(1)[Nle]NKKC(2)KC(3)YP |
| | | C(4)-NH₂ |
| 83 | 86 | H- |
| | | P[Nle]D[Nle]RC(1)SASVEC(2)KQKC(3)LKAIGC(4)IFGKC(1)[Nle]NKKC(2)KC(3)YP |
| | | C(4)-OH |
| 84 | 87 | H- |
| | | S[Nle]D[Nle]RC(1)SASVEC(2)KEKC(3)LQAIGSIFGKC(1)[Nle]NKKC(2)KC(3)YPR- |
| | | NH₂ |
| 85 | 88 | H-P[Nle]D[Nle]RC(1)SASVEC(2)KEKC(3)L[homo- |
| | | Gln]AIGSIFGKC(1)[Nle]NKKC(2)KC(3)YPR-NH₂ |
| 86 | 89 | H-C(1)SASVEC(2)KQKC(3)LKAIGC(4)IFGKC(1)[Nle]NKKC(2)KC(3)YPC(4)-NH₂ |
| 87 | 90 | H- |
| | | S[Nle]D[Nle]RC(1)SASVEC(2)KQKC(3)LAAIGC(4)IFGKC(1)[Nle]NKKC(2)KC(3)YP |
| | | C(4)-NH₂ |
| 88 | 91 | H-P[Nle]E[Nle]RC(1)[2-Amino-3- |
| | | guanidinopropionyl]ASVEC(2)KQKC(3)LAAIGSIFGKC(1)[Nle]NKKC(2)KC(3)YPR- |
| | | OH |
| 89 | 92 | H-S[Nle]D[Nle]RC(1)SALVEC(2)KQKC(3)LKAIGSIFGKC(1)MNKKC(2)KC(3)YPR- |
| | | NH₂ |
| 90 | 93 | H-S[Nle]D[Nle]RC(1)SAVVEC(2)KQKC(3)LKAIGSIFGKC(1)MNKKC(2)KC(3)YPR- |
| | | NH₂ |
| 91 | 94 | H-S[Nle]D[Nle]RC(1)SASVEC(2)KQKC(3)LKAIGSIFGKC(1)MNKKC(2)KC(3)[F(4- |
| | | F)]PR-NH₂ |
| 92 | 95 | H-S[Nle]D[Nle]RC(1)SASVEC(2)KQKC(3)LKAIGSIFGKC(1)MNKKC(2)KC(3)[F(4- |
| | | NO₂)]PR-NH₂ |
| 93 | 96 | H-S[Nle]D[Nle]RC(1)SASVEC(2)KQKC(3)LKAIGSIFGKC(1)MNKKC(2)KC(3)[F(4- |
| | | CH₃)]PR-NH₂ |
| 94 | 97 | H-[Nle]D[Nle]RC(1)SASVEC(2)KQKC(3)LKAIGSIFGKC(1)[Nle]NKKC(2)KC(3)YPR- |
| | | OH |
| 95 | 98 | H-NID[Nle]RC(1)SASVEC(2)KQKC(3)LKAIGSIFGKC(1)[Nle]NKKC(2)KC(3)YPR- |
| | | OH |
| 96 | 99 | H-PIE[Nle]RC(1)SASVEC(2)KQKC(3)LKAIGSIFGKC(1)[Nle]NKKC(2)KC(3)YPR-OH |
| 97 | 100 | H- |
| | | P[Nle]E[Nle]RC(1)SASVEC(2)KQKC(3)LAAIGSIFGKC(1)[Nle]NKKC(2)KC(3)YPR- |
| | | OH |
| 97 | 101 | H- |
| | | P[Nle]E[Nle]RC(1)SASVEC(2)KQKC(3)LAAIGSIFGKC(1)[Nle]NKKC(2)KC(3)YPR- |
| | | NH₂ |
| 98 | 102 | H- |
| | | P[Nle]E[Nle]RC(1)SA[Orn]VEC(2)KQKC(3)LAAIGSIFGKC(1)[Nle]NKKC(2)KC(3)YP |
| | | R-OH |
| 99 | 103 | H- |
| | | P[Nle]E[Nle]RC(1)SASVEC(2)KQKC(3)LLAIGSIFGKC(1)[Nle]NKKC(2)KC(3)YPR- |
| | | OH |
| 100 | 104 | H-PIDERC(1)SASVEC(2)KQKC(3)LAAIGSIFGKC(1)[Nle]NKKC(2)KC(3)YPR-OH |
| 101 | 105 | H-PIE[Nle]RC(1)SASVEC(2)KQKC(3)LAAIGSIFGKC(1)[Nle]NKKC(2)KC(3)YPR-OH |
| 102 | 106 | H-P[Nle]D[Nle]RC(1)SASVEC(2)AQKC(3)LAAIGSIFGKC(1)MNKKC(2)KC(3)YPR- |
| | | OH |
| 103 | 107 | H- |
| | | P[Nle]E[Nle]RC(1)SASVEC(2)AQKC(3)LAAIGSIFGKC(1)[Nle]NKKC(2)KC(3)YPR- |
| | | OH |
| 104 | 108 | H-C(1)SASVEC(2)KQKC(3)LKAIGSIFGKC(1)[Nle]NKKC(2)KC(3)YPR-NH₂ |
| 104 | 109 | Ac-C(1)SASVEC(2)KQKC(3)LKAIGSIFGKC(1)[Nle]NKKC(2)KC(3)YPR-NH₂ |
| 105 | 110 | H-P[Nle]E[Nle]RC(1)[2,3- |
| | | Diaminopropanoyl]ASVEC(2)KQKC(3)LAAIGSIFGKC(1)[Nle]NKKC(2)KC(3)YPR- |
| | | OH |
| 106 | 111 | H-RC(1)SASVEC(2)KQKC(3)LKAIGSIFGKC(1)[Nle]NKKC(2)KC(3)YPR-NH₂ |
| 106 | 112 | Ac-RC(1)SASVEC(2)KQKC(3)LKAIGSIFGKC(1)[Nle]NKKC(2)KC(3)YPR-OH |
| 107 | 113 | Ac-SKC(1)SASVEC(2)KQKC(3)LKAIGSIFGKC(1)[Nle]NKKC(2)KC(3)YPR-NH₂ |
| 108 | 114 | Ac-LRC(1)SASVEC(2)KQKC(3)LKAIGSIFGKC(1)[Nle]NKKC(2)KC(3)YPR-NH₂ |
| 108 | 115 | H-LRC(1)SASVEC(2)KQKC(3)LKAIGSIFGKC(1)[Nle]NKKC(2)KC(3)YPR-OH |
| 109 | 116 | Ac-C(1)SASVEC(2)KQKC(3)LKAIGSIFGKC(1)[Nle]NKKC(2)KC(3)YPS-NH₂ |
| 110 | 117 | Ac-C(1)SASVEC(2)KQKC(3)LKAIGSIFGKC(1)[Nle]NKKC(2)KC(3)YS-NH₂ |
| 111 | 118 | Ac-C(1)SASVEC(2)KQKC(3)LKAIGSIFGKC(1)[Nle]NKKC(2)KC(3)YG-NH₂ |
| 112 | 119 | Ac-C(1)SASVEC(2)KQKC(3)LKAIGSIFGKC(1)[Nle]NKKC(2)KC(3)Y-NH₂ |
| 113 | 120 | H- |
| | | P[Nle]E[Nle]RC(1)[Orn]ASVEC(2)KQKC(3)LAAIGSIFGKC(1)[Nle]NKKC(2)KC(3)YP |
| | | R-OH |
| 114 | 121 | H- |
| | | P[Nle]E[Nle]RC(1)HASVEC(2)KQKC(3)LAAIGSIFGKC(1)[Nle]NKKC(2)KC(3)YPR- |
| | | OH |
| 115 | 122 | Ac-LRC(1)SASVEC(2)KQKC(3)LAAIGSIFGKC(1)[Nle]NKKC(2)KC(3)YPR-OH |
| 116 | 123 | Ac-LRC(1)SASVEC(2)KQKC(3)LAAIGSIFGKC(1)MNKKC(2)KC(3)YPR-OH |
| 117 | 124 | H-C(1)SASVEC(2)KQKC(3)LKAIGSIFGKC(1)MNKKC(2)KC(3)YPR-OH |
| 118 | 125 | H- |
| | | P[Nle]E[Nle]RC(1)SASVEC(2)KEKC(3)LAAIGSIFGKC(1)[Nle]NKKC(2)KC(3)YPR- |
| | | OH |
| 119 | 126 | H- |
| | | p[Nle]E[Nle]RC(1)SASVEC(2)KQKC(3)LAAIGSIFGKC(1)[Nle]NKKC(2)KC(3)YPR- |
| | | OH |
| 120 | 127 | H-P[Nle]E[Nle]RC(1)SASVEC(2)KQKC(3)LAAIGSIFGKC(1)[Nle]NKKC(2)KC(3)YR- |
| | | OH |
| 121 | 128 | H-[Nle]E[Nle]RC(1)SASVEC(2)KQKC(3)LAAIGSIFGKC(1)[Nle]NKKC(2)KC(3)YPR- |
| | | OH |
| 122 | 129 | H- |
| | | P[Nle]E[Nle]RC(1)SASVEC(2)KQKC(3)LAAIGSIFGKC(1)[Nle]NKKC(2)KC(3)YPRR- |
| | | OH |
| 123 | 130 | H- |
| | | P[Nle]E[Nle]RC(1)SASVEC(2)KQKC(3)LAAIGSIFGKC(1)[Nle]NKKC(2)KC(3)YPRY- |
| | | OH |
| 124 | 131 | H- |
| | | P[Nle]E[Nle]RC(1)SASVEC(2)KQKC(3)LAAIGSIFGKC(1)[Nle]NKKC(2)KC(3)YPRL- |
| | | OH |
| 125 | 132 | H- |
| | | P[Nle]E[Nle]RC(1)SASVEC(2)KQKC(3)LAAIGSIFGKC(1)[Nle]NKKC(2)KC(3)YPRH- |
| | | OH |
| 126 | 133 | H- |
| | | P[Nle]E[Nle]RC(1)SASVEC(2)KQKC(3)LAAIGSIFGKC(1)[Nle]NKKC(2)KC(3)YPRE- |
| | | OH |
| 127 | 134 | H- |
| | | P[Nle]E[Nle]RC(1)SASVEC(2)KQKC(3)LAAIGSIFGKC(1)[Nle]NKKC(2)KC(3)YPRK |
| | | S-OH |
| 128 | 135 | H- |
| | | P[Nle]E[Nle]RC(1)SASVEC(2)KQKC(3)LAAIGSIFGKC(1)[Nle]NKKC(2)KC(3)YPRF |
| | | E-OH |
| 129 | 136 | H- |
| | | P[Nle]E[Nle]RC(1)SASVEC(2)KQKC(3)LAAIGSIFGKC(1)[Nle]NKKC(2)KC(3)YPRH |
| | | R-OH |
| 130 | 137 | H- |
| | | P[Nle]E[Nle]RC(1)SASVEC(2)KQKC(3)LAAIGSIFGKC(1)[Nle]NKKC(2)KC(3)YPRA |
| | | K-OH |
| 131 | 138 | H-P[Nle]E[Nle]RC(1)SASVEC(2)KQKC(3)LAAIGSIFGKC(1)[Nle]NKKC(2)KC(3)YP- |
| | | [(4-amino-5-hydroxypentyl)guanidine] |
| 132 | 139 | H- |
| | | P[Nle]E[Nle]RC(1)SASVEC(2)KQKC(3)LAAIGSIFGKC(1)[Nle]NKKC(2)KC(3)YPR- |
| | | [4-amino-5-hydroxypentanamide] |
| 133 | 140 | H- |
| | | P[Nle]E[Nle]RC(1)SASVEC(2)KQKC(3)LAAIGSIFGKC(1)[Nle]NKKC(2)KC(3)YPRS |
| | | T-OH |
| 134 | 141 | H- |
| | | P[Nle]E[Nle]RC(1)SASVEC(2)KQKC(3)LAAIGSIFGKC(1)[Nle]NKKC(2)KC(3)YPRR |
| | | Y-OH |
| 135 | 142 | H- |
| | | P[Nle]E[Nle]RC(1)SSSVEC(2)KQKC(3)LAAIGSIFGKC(1)[Nle]NKKC(2)KC(3)YPR- |
| | | OH |
| 136 | 143 | H- |
| | | P[Nle]E[Nle]RC(1)SLSVEC(2)KQKC(3)LAAIGSIFGKC(1)[Nle]NKKC(2)KC(3)YPR- |
| | | OH |
| 137 | 144 | H- |
| | | P[Nle]E[Nle]RC(1)SAPVEC(2)KQKC(3)LAAIGSIFGKC(1)[Nle]NKKC(2)KC(3)YPR- |
| | | OH |
| 138 | 145 | H- |
| | | P[Nle]E[Nle]RC(1)SASPEC(2)KQKC(3)LAAIGSIFGKC(1)[Nle]NKKC(2)KC(3)YPR- |
| | | OH |
| 139 | 146 | H- |
| | | P[Nle]E[Nle]RC(1)SASQEC(2)KQKC(3)LAAIGSIFGKC(1)[Nle]NKKC(2)KC(3)YPR- |
| | | OH |
| 140 | 147 | H- |
| | | P[Nle]E[Nle]RC(1)SASVEC(2)LQKC(3)LAAIGSIFGKC(1)[Nle]NKKC(2)KC(3)YPR- |
| | | OH |
| 141 | 148 | H- |
| | | P[Nle]E[Nle]RC(1)SASVEC(2)KQPC(3)LAAIGSIFGKC(1)[Nle]NKKC(2)KC(3)YPR- |
| | | OH |
| 142 | 149 | H- |
| | | P[Nle]E[Nle]RC(1)EASVEC(2)KQKC(3)LAAIGSIFGKC(1)[Nle]NKKC(2)KC(3)YPR- |
| | | OH |
| 143 | 150 | H- |
| | | P[Nle]E[Nle]RC(1)FASVEC(2)KQKC(3)LAAIGSIFGKC(1)[Nle]NKKC(2)KC(3)YPR- |
| | | OH |
| 144 | 151 | H- |
| | | P[Nle]E[Nle]RC(1)SYSVEC(2)KQKC(3)LAAIGSIFGKC(1)[Nle]NKKC(2)KC(3)YPR- |
| | | OH |
| 145 | 152 | H- |
| | | P[Nle]E[Nle]RC(1)SAFVEC(2)KQKC(3)LAAIGSIFGKC(1)[Nle]NKKC(2)KC(3)YPR- |
| | | OH |
| 146 | 153 | H-P[Nle]E[Nle]RC(1)SA[2,3- |
| | | Diaminopropanoyl]VEC(2)KQKC(3)LAAIGSIFGKC(1)[Nle]NKKC(2)KC(3)YPR-OH |
| 147 | 154 | H-P[Nle]E[Nle]RC(1)[2,4- |
| | | Diaminobutanoyl]ASVEC(2)AQSC(3)LAAIGSIFGKC(1)[Nle]NGKC(2)KC(3)YPR-OH |
| 147 | 155 | H-P[Nle]E[Nle]RC(1)[2,4- |
| | | Diaminobutanoyl]ASVEC(2)AQSC(3)LAAIGSIFGKC(1)[Nle]NGKC(2)KC(3)YPR- |
| | | NH₂ |
| 148 | 156 | H- |
| | | P[Nle]E[Nle]RC(1)RASVEC(2)AQSC(3)LAAIGSIFGKC(1)[Nle]NGKC(2)KC(3)YPR- |
| | | NH₂ |
| 149 | 157 | H- |
| | | P[Nle]E[Nle]RC(1)SASREC(2)AQSC(3)LAAIGSIFGKC(1)[Nle]NGKC(2)KC(3)YPR- |
| | | NH₂ |
| 150 | 158 | H- |
| | | P[Nle]E[Nle]RC(1)SASVEC(2)KQKC(3)LAKIGSIFGKC(1)[Nle]NKKC(2)KC(3)YPR- |
| | | OH |

10. The Kv1.3 inhibitor or pharmaceutically acceptable salt for use according to any one of claims 1 to 9, wherein the disease or disorder is
(a) an inflammatory disease or disorder, preferably wherein the inflammatory disease or disorder is selected from an autoimmune disorder, an allergy or hypersensitivity, allograft rejection, transplant rejection, graft-versus-host disease, hay fever, asthma, anaphylaxis, allergic rhinitis, urticaria, eczema, alopecia areata, dermatomyositis, inclusion body myositis, polymyositis, ankylosing spondylitis, vasculitis, arthritis (including rheumatoid arthritis, osteoarthritis, psoriatic arthritis), Sjogren's syndrome, systemic lupus erythematosus (SLE), uveitis, inflammatory fibrosis (e.g. scleroderma, lung fibrosis, cirrhosis), chronic obstructive pulmonary disease (COPD), hepatitis, chronic inflammatory demyelinating polyneuropathy, inflammatory bowel disease, colitis (e.g. Crohn's disease and ulcerative colitis), erythema, thyroiditis, psoriasis, atopic dermatitis, allergic contact dermatitis, scleroderma, glomerulonephritis, inflammatory bone resorption, multiple sclerosis and type 1 diabetes; or
(b) a metabolic disease or disorder; or
(c) selected from obesity, obesity-linked inflammation, obesity-linked gall bladder disease and obesity-induced sleep apnoea; or
(d) caused by or associated with impaired glucose control, preferably wherein the disease or disorder is selected from metabolic syndrome, insulin resistance, glucose intolerance, pre-diabetes, increased fasting glucose and type 2 diabetes; or
(e) a smooth muscle proliferative disorder, preferably wherein the smooth muscle proliferative disorder is restenosis; or
(f) a neuroinflammatory or neurodegenerative disease or disorder, preferably wherein the neuroinflammatory or neurodegenerative disease or disorder is selected from Alzheimer's disease, multiple sclerosis (MS), Parkinson's disease and amyotrophic lateral sclerosis (ALS); or
(g) cancer, preferably wherein the cancer is breast cancer, prostate cancer or lymphoma, preferably wherein the lymphoma is non-Hodgkin lymphoma (NHL), preferably wherein the NHL is selected from diffuse large B-cell lymphoma, follicular lymphoma, Burkitt lymphoma, immunoblastic large cell lymphoma, precursor B-lymphoblastic lymphoma, mantle cell lymphoma, mycosis fungoides, anaplastic large cell lymphoma, peripheral T-cell lymphoma, precursor T-lymphoblastic lymphoma and Sezary syndrome.

11. A Kv1.3 inhibitor, or a pharmaceutically acceptable salt thereof, as defined in any one of claims 1 to 9, for use in a method of
(a) inhibiting or reducing inflammation; or
(b) inhibiting weight gain, promoting weight loss, reducing excess body weight or treating obesity;
wherein the method comprises administering the Kv1.3 inhibitor to the subject once every 2 to 8 days.

12. The Kv1.3 inhibitor or a pharmaceutically acceptable salt for use according to any one of claims 1 to 11, wherein the method comprises administering the Kv1.3 inhibitor to the subject once every 2 to 7 days, once every 2 to 6 days, once every 2 to 5 days, once every 2 to 4 days, once every 2 to 3 days, once every 3 to 8 days, once every 3 to 7 days, once every 3 to 6 days, once every 3 to 5 days, once every 3 to 4 days, once every 4 to 8 days, once every 4 to 7 days, once every 4 to 6 days, once every 4 to 5 days, once every 5 to 8 days, once every 5 to 7 days, once every 5 to 6 days, once every 6 to 8 days, once every 6 to 7 days or once every 7 to 8 days.

13. The Kv1.3 inhibitor or pharmaceutically acceptable salt for use according to any one of claims 1 to 12, wherein the method comprises administering the Kv1.3 inhibitor to the subject once every 2 days, once every 3 days, once every 4 days, once every 5 days, once every 6 days, once every 7 days or once every 8 days, preferably once every 7 days.

14. The Kv1.3 inhibitor or pharmaceutically acceptable salt for use according to any one of claims 1 to 13, wherein the method comprises administering the Kv1.3 inhibitor to the subject by injection, preferably by subcutaneous injection.

15. The Kv1.3 inhibitor or pharmaceutically acceptable salt for use according to any one of claims 1 to 14, wherein the method comprises administering the Kv1.3 inhibitor to the subject at a dosage of 0.1 mg to 30.0 mg.

16. The Kv1.3 inhibitor or pharmaceutically acceptable salt for use according to any one of claims 1 to 15, wherein the method comprises administering the Kv1.3 inhibitor to the subject at a dosage of 10 nmol/kg to 400 nmol/kg or at a dosage of 3 nmol/kg to 300 nmol/kg.

17. The Kv1.3 inhibitor or pharmaceutically acceptable salt for use according to any one of claims 1 to 16, wherein the Kv1.3 inhibitor or pharmaceutically acceptable salt is in the form of a composition, preferably wherein the composition is a pharmaceutical composition, and preferably wherein the pharmaceutical composition comprises a pharmaceutically acceptable carrier, excipient or vehicle.

18. The Kv1.3 inhibitor or pharmaceutically acceptable salt for use according to claim 17, wherein the composition comprises one or more peptides wherein each of the peptides comprises or consists of a sequence independently selected from the following sequences:

| SEQ | |
|---|---|
| ID NO | Sequence |
| 1 | QMDMRCSASVECKQKCLKAIGSIFGKCMNKKCKCYPR |
| 2 | QMDMRCSASVECKQKCLKAIGRGFGKCMNKKCKCYPR |
| 3 | NMDMRCSASVECKQKCLKAIGSIFGKCMNKKCKCYPR |
| 4 | NMDMRCSASVECKQKCLKAIGRGFGKCMNKKCKCYPR |
| 5 | MDMRCSASVECKQKCLKAIGSIFGKCMNKKCKCYPR |
| 6 | DMRCSASVECKQKCLKAIGSIFGKCMNKKCKCYPR |
| 7 | P[Nle]E[Nle]RCYASVECKQKCLAAIGSIFGKC[Nle]NKKCKCYPR |
| 8 | NIDMRCSASVECKQKCLKAIGSIFGKCMNKKCKCYPR |
| 9 | NMDVRCSASVECKQKCLKAIGSIFGKCMNKKCKCYPR |
| 10 | NMDMRCSASVECKQKCKDAIGSIFGKCMNKKCKCYPR |
| 11 | GGNMDMRCSASVECKQKCLKAIGSIFGKCMNKKCKCYPR |
| 12 | NMEMRCSASVECKQKCLKAIGSIFGKCMNKKCKCYPR |
| 13 | SGNMDMRCSASVECKQKCLKAIGSIFGKCMNKKCKCYPR |
| 14 | N[Nle]DMRCSASVECKQKCLKAIGSIFGKCMNKKCKCYPR |
| 15 | NMD[Nle]RCSASVECKQKCLKAIGSIFGKCMNKKCKCYPR |
| 16 | NMDMRCSASVECKQKCLKAIGSIFGKC[Nle]NKKCKCYPR |
| 17 | NMDMRCSASVECKVKCLKAIGSIFGKCMNKKCKCYPR |
| 18 | NMDMRCSASVECKQLCLKAIGSIFGKCMNKKCKCYPR |
| 19 | NMDMRCSASVECKQKCKKAIGSIFGKCMNKKCKCYPR |
| 20 | NMDMRCSASVECKQKCLDAIGSIFGKCMNKKCKCYPR |
| 21 | NMDMRCSASVECKQKCLKAIRSIFGKCMNKKCKCYPR |
| 22 | NMDMRCSASVECKQKCLKAIESIFGKCMNKKCKCYPR |
| 23 | NMDMRCSASVECKQKCLKAIGSIFGKCMNKKCKCYPRRRTA |
| 24 | NMDMRCSASVECKQKCLKAIGSIFGKCMNKKCKCYPRHRRK |
| 25 | NMDMRCKASVECKQKCLKAIGSIFGKCMNKKCKCYPR |
| 26 | NMDMRCSISVECKQKCLKAIGSIFGKCMNKKCKCYPR |
| 27 | NMDMRCSASRECKQKCLKAIGSIFGKCMNKKCKCYPR |
| 28 | NMDMRCSASVQCKQKCLKAIGSIFGKCMNKKCKCYPR |
| 29 | NMDMRCSASVECLQKCLKAIGSIFGKCMNKKCKCYPR |
| 30 | NMDMRCSASVECAQKCLKAIGSIFGKCMNKKCKCYPR |
| 31 | NMDMRCSASVECKEKCLKAIGSIFGKCMNKKCKCYPR |
| 32 | NMDMRCSASVECKLKCLKAIGSIFGKCMNKKCKCYPR |
| 33 | NMDMRCSASVECKQKCLKAIHSIFGKCMNKKCKCYPR |
| 34 | NMDMRCSASVECKQKCLKAIGSKFGKCMNKKCKCYPR |
| 35 | NMDMRCSASVECKQKCLKAIGSRFGKCMNKKCKCYPR |
| 36 | NMDMRCSASVECKQKCLKAIGSIFGKCMNGKCKCYPR |
| 37 | NMDMRCSASVECKQKCLKAIGSIFGKCMNKKCHCYPR |
| 38 | NMDMRCSASVECKQKCLKAIGSIFGKCMNKKCVCYPR |
| 39 | N[Nle]D[Nle]RCSASVECKQKCLKAIGSIFGKCMNKKCKCYPR |
| 40 | N[Nle]D[Nle]RCSASVECKQKCLKAIGSIFGKC[Nle]NKKCKCYPR |
| 41 | P[Nle]D[Nle]RCSASVECKQKCLKAIGSIFGKC[Nle]NKKCKCYPR |
| 42 | N[Nle]D[Nle]RCRASVECKQKCLKAIGSIFGKC[Nle]NKKCKCYPR |
| 43 | N[Nle]D[Nle]RCSASVECEQKCLKAIGSIFGKC[Nle]NKKCKCYPR |
| 44 | N[Nle]D[Nle]RCSASVECQQKCLKAIGSIFGKC[Nle]NKKCKCYPR |
| 45 | N[Nle]D[Nle]RCSASVECKKKCLKAIGSIFGKC[Nle]NKKCKCYPR |
| 46 | N[Nle]S[Nle]RCSASVECKQKCLKAIGSIFGKC[Nle]NKKCKCYPR |
| 47 | N[Nle]D[Nle]RCSHSVECKQKCLKAIGSIFGKC[Nle]NKKCKCYPR |
| 48 | N[Nle]D[Nle]RCSASVECKQSCLKAIGSIFGKC[Nle]NKKCKCYPR |
| 49 | N[Nle]D[Nle]RCSASVECKQKCKAIGSIFGKC[Nle]NKKCKCYPR |
| 50 | NMDMRCSASVECKQKCYKAIGSIFGKCMNKKCKCYPR |
| 51 | NMDMRCSASVECKQKCRKAIGSIFGKCMNKKCKCYPR |
| 52 | NMDMRCSASVECKQKCLAAIGSIFGKCMNKKCKCYPR |
| 53 | NMDMRCSASVECKQKCLYAIGSIFGKCMNKKCKCYPR |
| 54 | NMDMRCSASVECKQKCLAIGSIFGKCMNKKCKCYPR |
| 55 | NMDMRCSASVECKQKCLKYIGSIFGKCMNKKCKCYPR |
| 56 | NMDMRCSASVECKQKCLKRIGSIFGKCMNKKCKCYPR |
| 57 | NMDMRCSASVECKQKCLKIGSIFGKCMNKKCKCYPR |
| 58 | NMDMRCSASVECKQKCLKAYGSIFGKCMNKKCKCYPR |
| 59 | NMDMRCSASVECKQKCLKAEGSIFGKCMNKKCKCYPR |
| 60 | NMDMRCSASVECKQKCLKARGSIFGKCMNKKCKCYPR |
| 61 | NMDMRCSASVECKQKCLKAGSIFGKCMNKKCKCYPR |
| 62 | N[Nle]D[Nle]RCSASVECKQKCLKAIGSPFGKC[Nle]NKKCKCYPR |
| 63 | N[Nle]D[Nle]RCSASKECKQKCLKAIGSIFGKC[Nle]NKKCKCYPR |
| 64 | H[Nle]D[Nle]RCSASVECKQKCLKAIGSIFGKC[Nle]NKKCKCYPR |
| 65 | Y[Nle]D[Nle]RCSASVECKQKCLKAIGSIFGKC[Nle]NKKCKCYPR |
| 66 | S[Nle]D[Nle]RCSASVECKQKCLKAIGSIFGKC[Nle]NKKCKCYPR |
| 67 | V[Nle]D[Nle]RCSASVECKQKCLKAIGSIFGKC[Nle]NKKCKCYPR |
| 68 | S[Nle]D[Nle]RCSA[Abu]VECKQKCLKAIGSIFGKCMNKKCKCYPR |
| 69 | S[Nle]D[Nle]RCSASVECKQKCLKAIGSIFG[homo-Lys]CMNKKCKCYPR |
| 70 | S[Nle]D[Nle]RCSASVECKQKCLKAIGSIFGKCMNKKCKC[F(4-NH2)]PR |
| 71 | S[Nle]D[Nle]RCSASVECGQKCLKAIGSIFGKCMNKKCKCYPR |
| 72 | S[Nle]D[Nle]RCSASVECVQKCLKAIGSIFGKCMNKKCKCYPR |
| 73 | S[Nle]D[Nle]RCSASVECK[2-Amino-5- |
| | carboxypentanoyl]KCLKAIGSIFGKCMNKKCKCYPR |
| 74 | S[Nle]D[Nle]RCSASVECKQKCLKAIGSIFGKCMNKKCKCYQ |
| 75 | S[Nle]D[Nle]RCSASVECKQKCLKAIGSIFGKCMNKKCRCYPR |
| 76 | S[Nle]DERCSASVECKQKCLKAIGSIFGKCMNKKCKCYPR |
| 77 | S[Nle]D[Nle]RCSASVECKQKCLGAIGSIFGKCMNKKCKCYPR |
| 78 | S[Nle]D[Nle]RCSASVECKQKCLVAIGSIFGKCMNKKCKCYPR |
| 79 | S[Nle]D[Nle]RCSASVECAQSCLKAIGSIFGKCMNKKCKCYPR |
| 80 | S[Nle]D[Nle]RCSASVECAQKCLAAIGSIFGKCMNKKCKCYPR |
| 81 | S[Nle]D[Nle]RCSASVECAQLCLAAIGSIFGKCMNKKCKCYPR |
| 82 | P[Nle]E[Nle]RC[2,4- |
| | Diaminobutanoyl]ASVECKQKCLAAIGSIFGKC[Nle]NKKCKCYPR |
| 83 | P[Nle]D[Nle]RCSASVECKQKCLKAIGCIFGKC[Nle]NKKCKCYPC |
| 84 | S[Nle]D[Nle]RCSASVECKEKCLQAIGSIFGKC[Nle]NKKCKCYPR |
| 85 | P[Nle]D[Nle]RCSASVECKEKCL[homo-Gln]AIGSIFGKC[Nle]NKKCKCYPR |
| 86 | CSASVECKQKCLKAIGCIFGKC[Nle]NKKCKCYPC |
| 87 | S[Nle]D[Nle]RCSASVECKQKCLAAIGCIFGKC[Nle]NKKCKCYPC |
| 88 | P[Nle]E[Nle]RC[2-Amino-3- |
| | guanidinopropionyl]ASVECKQKCLAAIGSIFGKC[Nle]NKKCKCYPR |
| 89 | S[Nle]D[Nle]RCSALVECKQKCLKAIGSIFGKCMNKKCKCYPR |
| 90 | S[Nle]D[Nle]RCSAVVECKQKCLKAIGSIFGKCMNKKCKCYPR |
| 91 | S[Nle]D[Nle]RCSASVECKQKCLKAIGSIFGKCMNKKCKC[F(4-F)]PR |
| 92 | S[Nle]D[Nle]RCSASVECKQKCLKAIGSIFGKCMNKKCKC[F(4-NO₂)]PR |
| 93 | S[Nle]D[Nle]RCSASVECKQKCLKAIGSIFGKCMNKKCKC[F(4-CH₃)]PR |
| 94 | [Nle]D[Nle]RCSASVECKQKCLKAIGSIFGKC[Nle]NKKCKCYPR |
| 95 | NID[Nle]RCSASVECKQKCLKAIGSIFGKC[Nle]NKKCKCYPR |
| 96 | PIE[Nle]RCSASVECKQKCLKAIGSIFGKC[Nle]NKKCKCYPR |
| 97 | P[Nle]E[Nle]RCSASVECKQKCLAAIGSIFGKC[Nle]NKKCKCYPR |
| 98 | P[Nle]E[Nle]RCSA[Orn]VECKQKCLAAIGSIFGKC[Nle]NKKCKCYPR |
| 99 | P[Nle]E[Nle]RCSASVECKQKCLLAIGSIFGKC[Nle]NKKCKCYPR |
| 100 | PIDERCSASVECKQKCLAAIGSIFGKC[Nle]NKKCKCYPR |
| 101 | PIE[Nle]RCSASVECKQKCLAAIGSIFGKC[Nle]NKKCKCYPR |
| 102 | P[Nle]D[Nle]RCSASVECAQKCLAAIGSIFGKCMNKKCKCYPR |
| 103 | P[Nle]E[Nle]RCSASVECAQKCLAAIGSIFGKC[Nle]NKKCKCYPR |
| 104 | CSASVECKQKCLKAIGSIFGKC[Nle]NKKCKCYPR |
| 105 | P[Nle]E[Nle]RC(1)[2,3- |
| | Diaminopropanoyl]ASVEC(2)KQKC(3)LAAIGSIFGKC(1)[Nle]NKKC(2)KC(3)YPR |
| 106 | RCSASVECKQKCLKAIGSIFGKC[Nle]NKKCKCYPR |
| 107 | SKCSASVECKQKCLKAIGSIFGKC[Nle]NKKCKCYPR |
| 108 | LRCSASVECKQKCLKAIGSIFGKC[Nle]NKKCKCYPR |
| 109 | CSASVECKQKCLKAIGSIFGKC[Nle]NKKCKCYPS |
| 110 | CSASVECKQKCLKAIGSIFGKC[Nle]NKKCKCYS |
| 111 | CSASVECKQKCLKAIGSIFGKC[Nle]NKKCKCYG |
| 112 | CSASVECKQKCLKAIGSIFGKC[Nle]NKKCKCY |
| 113 | P[Nle]E[Nle]RC[Orn]ASVECKQKCLAAIGSIFGKC[Nle]NKKCKCYPR |
| 114 | P[Nle]E[Nle]RCHASVECKQKCLAAIGSIFGKC[Nle]NKKCKCYPR |
| 115 | LRCSASVECKQKCLAAIGSIFGKC[Nle]NKKCKCYPR |
| 116 | LRCSASVECKQKCLAAIGSIFGKCMNKKCKCYPR |
| 117 | CSASVECKQKCLKAIGSIFGKCMNKKCKCYPR |
| 118 | P[Nle]E[Nle]RCSASVECKEKCLAAIGSIFGKC[Nle]NKKCKCYPR |
| 119 | p[Nle]E[Nle]RCSASVECKQKCLAAIGSIFGKC[Nle]NKKCKCYPR |
| 120 | P[Nle]E[Nle]RCSASVECKQKCLAAIGSIFGKC[Nle]NKKCKCYR |
| 121 | [Nle]E[Nle]RCSASVECKQKCLAAIGSIFGKC[Nle]NKKCKCYPR |
| 122 | P[Nle]E[Nle]RCSASVECKQKCLAAIGSIFGKC[Nle]NKKCKCYPRR |
| 123 | P[Nle]E[Nle]RCSASVECKQKCLAAIGSIFGKC[Nle]NKKCKCYPRY |
| 124 | P[Nle]E[Nle]RCSASVECKQKCLAAIGSIFGKC[Nle]NKKCKCYPRL |
| 125 | P[Nle]E[Nle]RCSASVECKQKCLAAIGSIFGKC[Nle]NKKCKCYPRH |
| 126 | P[Nle]E[Nle]RCSASVECKQKCLAAIGSIFGKC[Nle]NKKCKCYPRE |
| 127 | P[Nle]E[Nle]RCSASVECKQKCLAAIGSIFGKC[Nle]NKKCKCYPRKS |
| 128 | P[Nle]E[Nle]RCSASVECKQKCLAAIGSIFGKC[Nle]NKKCKCYPRFE |
| 129 | P[Nle]E[Nle]RCSASVECKQKCLAAIGSIFGKC[Nle]NKKCKCYPRHR |
| 130 | P[Nle]E[Nle]RCSASVECKQKCLAAIGSIFGKC[Nle]NKKCKCYPRAK |
| 131 | P[Nle]E[Nle]RCSASVECKQKCLAAIGSIFGKC[Nle]NKKCKCYP-[(4-amino-5- |
| | hydroxypentyl)guanidine] |
| 132 | P[Nle]E[Nle]RCSASVECKQKCLAAIGSIFGKC[Nle]NKKCKCYPR-[4-amino-5- |
| | hydroxypentanamide] |
| 133 | P[Nle]E[Nle]RCSASVECKQKCLAAIGSIFGKC[Nle]NKKCKCYPRST |
| 134 | P[Nle]E[Nle]RCSASVECKQKCLAAIGSIFGKC[Nle]NKKCKCYPRRY |
| 135 | P[Nle]E[Nle]RCSSSVECKQKCLAAIGSIFGKC[Nle]NKKCKCYPR |
| 136 | P[Nle]E[Nle]RCSLSVECKQKCLAAIGSIFGKC[Nle]NKKCKCYPR |
| 137 | P[Nle]E[Nle]RCSAPVECKQKCLAAIGSIFGKC[Nle]NKKCKCYPR |
| 138 | P[Nle]E[Nle]RCSASPECKQKCLAAIGSIFGKC[Nle]NKKCKCYPR |
| 139 | P[Nle]E[Nle]RCSASQECKQKCLAAIGSIFGKC[Nle]NKKCKCYPR |
| 140 | P[Nle]E[Nle]RCSASVECLQKCLAAIGSIFGKC[Nle]NKKCKCYPR |
| 141 | P[Nle]E[Nle]RCSASVECKQPCLAAIGSIFGKC[Nle]NKKCKCYPR |
| 142 | P[Nle]E[Nle]RCEASVECKQKCLAAIGSIFGKC[Nle]NKKCKCYPR |
| 143 | P[Nle]E[Nle]RCFASVECKQKCLAAIGSIFGKC[Nle]NKKCKCYPR |
| 144 | P[Nle]E[Nle]RCSYSVECKQKCLAAIGSIFGKC[Nle]NKKCKCYPR |
| 145 | P[Nle]E[Nle]RCSAFVECKQKCLAAIGSIFGKC[Nle]NKKCKCYPR |
| 146 | P[Nle]E[Nle]RCSA[2,3- |
| | Diaminopropanoyl]VECKQKCLAAIGSIFGKC[Nle]NKKCKCYPR |
| 147 | P[Nle]E[Nle]RC[2,4- |
| | Diaminobutanoyl]ASVECAQSCLAAIGSIFGKC[Nle]NGKCKCYPR |
| 148 | P[Nle]E[Nle]RCRASVECAQSCLAAIGSIFGKC[Nle]NGKCKCYPR |
| 149 | P[Nle]E[Nle]RCSASRECAQSCLAAIGSIFGKC[Nle]NGKCKCYPR |
| 150 | P[Nle]E[Nle]RCSASVECKQKCLAKIGSIFGKC[Nle]NKKCKCYPR |

19. The Kv1.3 inhibitor or pharmaceutically acceptable salt for use according to claim 17 or claim 18, wherein the composition comprises one or more peptides wherein the peptides are independently selected from the following peptide sequences:

| SEQ | | |
|---|---|---|
| ID | Ptd | |
| NO | No. | Peptide |
| 1 | 1 | H-QMDMRC(1)SASVEC(2)KQKC(3)LKAIGSIFGKC(1)MNKKC(2)KC(3)YPR-OH |
| 2 | 2 | H-QMDMRC(1)SASVEC(2)KQKC(3)LKAIGRGFGKC(1)MNKKC(2)KC(3)YPR-OH |
| 3 | 3 | H-NMDMRC(1)SASVEC(2)KQKC(3)LKAIGSIFGKC(1)MNKKC(2)KC(3)YPR-OH |
| 3 | 4 | H-NMDMRC(1)SASVEC(2)KQKC(3)LKAIGSIFGKC(1)MNKKC(2)KC(3)YPR-NH₂ |
| 4 | 5 | H-NMDMRC(1)SASVEC(2)KQKC(3)LKAIGRGFGKC(1)MNKKC(2)KC(3)YPR-OH |
| 5 | 6 | H-MDMRC(1)SASVEC(2)KQKC(3)LKAIGSIFGKC(1)MNKKC(2)KC(3)YPR-OH |
| 6 | 7 | H-DMRC(1)SASVEC(2)KQKC(3)LKAIGSIFGKC(1)MNKKC(2)KC(3)YPR-OH |
| 7 | 8 | H- |
| | | P[Nle]E[Nle]RC(1)YASVEC(2)KQKC(3)LAAIGSIFGKC(1)[Nle]NKKC(2)KC(3)YPR- |
| | | OH |
| 8 | 9 | H-NIDMRC(1)SASVEC(2)KQKC(3)LKAIGSIFGKC(1)MNKKC(2)KC(3)YPR-OH |
| 9 | 10 | H-NMDVRC(1)SASVEC(2)KQKC(3)LKAIGSIFGKC(1)MNKKC(2)KC(3)YPR-OH |
| 10 | 11 | H-NMDMRC(1)SASVEC(2)KQKC(3)KDAIGSIFGKC(1)MNKKC(2)KC(3)YPR-OH |
| 11 | 12 | H-GGNMDMRC(1)SASVEC(2)KQKC(3)LKAIGSIFGKC(1)MNKKC(2)KC(3)YPR-OH |
| 12 | 13 | H-NMEMRC(1)SASVEC(2)KQKC(3)LKAIGSIFGKC(1)MNKKC(2)KC(3)YPR-OH |
| 13 | 14 | H-SGNMDMRC(1)SASVEC(2)KQKC(3)LKAIGSIFGKC(1)MNKKC(2)KC(3)YPR-OH |
| 14 | 15 | H-N[Nle]DMRC(1)SASVEC(2)KQKC(3)LKAIGSIFGKC(1)MNKKC(2)KC(3)YPR-NH₂ |
| 15 | 16 | H-NMD[Nle]RC(1)SASVEC(2)KQKC(3)LKAIGSIFGKC(1)MNKKC(2)KC(3)YPR-NH₂ |
| 16 | 17 | H-NMDMRC(1)SASVEC(2)KQKC(3)LKAIGSIFGKC(1)[Nle]NKKC(2)KC(3)YPR-NH₂ |
| 17 | 18 | H-NMDMRC(1)SASVEC(2)KVKC(3)LKAIGSIFGKC(1)MNKKC(2)KC(3)YPR-NH₂ |
| 18 | 19 | H-NMDMRC(1)SASVEC(2)KQLC(3)LKAIGSIFGKC(1)MNKKC(2)KC(3)YPR-NH₂ |
| 19 | 20 | H-NMDMRC(1)SASVEC(2)KQKC(3)KKAIGSIFGKC(1)MNKKC(2)KC(3)YPR-NH₂ |
| 20 | 21 | H-NMDMRC(1)SASVEC(2)KQKC(3)LDAIGSIFGKC(1)MNKKC(2)KC(3)YPR-NH₂ |
| 21 | 22 | H-NMDMRC(1)SASVEC(2)KQKC(3)LKAIRSIFGKC(1)MNKKC(2)KC(3)YPR-NH₂ |
| 22 | 23 | H-NMDMRC(1)SASVEC(2)KQKC(3)LKAIESIFGKC(1)MNKKC(2)KC(3)YPR-NH₂ |
| 23 | 24 | H-NMDMRC(1)SASVEC(2)KQKC(3)LKAIGSIFGKC(1)MNKKC(2)KC(3)YPRRRTA- |
| | | NH₂ |
| 24 | 25 | H-NMDMRC(1)SASVEC(2)KQKC(3)LKAIGSIFGKC(1)MNKKC(2)KC(3)YPRHRRK- |
| | | NH₂ |
| 25 | 26 | H-NMDMRC(1)KASVEC(2)KQKC(3)LKAIGSIFGKC(1)MNKKC(2)KC(3)YPR-NH₂ |
| 26 | 27 | H-NMDMRC(1)SISVEC(2)KQKC(3)LKAIGSIFGKC(1)MNKKC(2)KC(3)YPR-NH₂ |
| 27 | 28 | H-NMDMRC(1)SASREC(2)KQKC(3)LKAIGSIFGKC(1)MNKKC(2)KC(3)YPR-NH₂ |
| 28 | 29 | H-NMDMRC(1)SASVQC(2)KQKC(3)LKAIGSIFGKC(1)MNKKC(2)KC(3)YPR-NH₂ |
| 29 | 30 | H-NMDMRC(1)SASVEC(2)LQKC(3)LKAIGSIFGKC(1)MNKKC(2)KC(3)YPR-NH₂ |
| 30 | 31 | H-NMDMRC(1)SASVEC(2)AQKC(3)LKAIGSIFGKC(1)MNKKC(2)KC(3)YPR-NH₂ |
| 31 | 32 | H-NMDMRC(1)SASVEC(2)KEKC(3)LKAIGSIFGKC(1)MNKKC(2)KC(3)YPR-NH₂ |
| 32 | 33 | H-NMDMRC(1)SASVEC(2)KLKC(3)LKAIGSIFGKC(1)MNKKC(2)KC(3)YPR-NH₂ |
| 33 | 34 | H-NMDMRC(1)SASVEC(2)KQKC(3)LKAIHSIFGKC(1)MNKKC(2)KC(3)YPR-NH₂ |
| 34 | 35 | H-NMDMRC(1)SASVEC(2)KQKC(3)LKAIGSKFGKC(1)MNKKC(2)KC(3)YPR-NH₂ |
| 35 | 36 | H-NMDMRC(1)SASVEC(2)KQKC(3)LKAIGSRFGKC(1)MNKKC(2)KC(3)YPR-NH₂ |
| 36 | 37 | H-NMDMRC(1)SASVEC(2)KQKC(3)LKAIGSIFGKC(1)MNGKC(2)KC(3)YPR-NH₂ |
| 37 | 38 | H-NMDMRC(1)SASVEC(2)KQKC(3)LKAIGSIFGKC(1)MNKKC(2)HC(3)YPR-NH₂ |
| 38 | 39 | H-NMDMRC(1)SASVEC(2)KQKC(3)LKAIGSIFGKC(1)MNKKC(2)VC(3)YPR-NH₂ |
| 39 | 40 | H-N[Nle]D[Nle]RC(1)SASVEC(2)KQKC(3)LKAIGSIFGKC(1)MNKKC(2)KC(3)YPR- |
| | | NH₂ |
| 40 | 41 | H- |
| | | N[Nle]D[Nle]RC(1)SASVEC(2)KQKC(3)LKAIGSIFGKC(1)[Nle]NKKC(2)KC(3)YPR- |
| | | NH₂ |
| 41 | 42 | H- |
| | | P[Nle]D[Nle]RC(1)SASVEC(2)KQKC(3)LKAIGSIFGKC(1)[Nle]NKKC(2)KC(3)YPR- |
| | | NH₂ |
| 42 | 43 | H- |
| | | N[Nle]D[Nle]RC(1)RASVEC(2)KQKC(3)LKAIGSIFGKC(1)[Nle]NKKC(2)KC(3)YPR- |
| | | NH₂ |
| 43 | 44 | H- |
| | | N[Nle]D[Nle]RC(1)SASVEC(2)EQKC(3)LKAIGSIFGKC(1)[Nle]NKKC(2)KC(3)YPR- |
| | | NH₂ |
| 44 | 45 | H- |
| | | N[Nle]D[Nle]RC(1)SASVEC(2)QQKC(3)LKAIGSIFGKC(1)[Nle]NKKC(2)KC(3)YPR- |
| | | NH₂ |
| 45 | 46 | H- |
| | | N[Nle]D[Nle]RC(1)SASVEC(2)KKKC(3)LKAIGSIFGKC(1)[Nle]NKKC(2)KC(3)YPR- |
| | | NH₂ |
| 46 | 47 | H- |
| | | N[Nle]S[Nle]RC(1)SASVEC(2)KQKC(3)LKAIGSIFGKC(1)[Nle]NKKC(2)KC(3)YPR- |
| | | NH₂ |
| 47 | 48 | H- |
| | | N[Nle]D[Nle]RC(1)SHSVEC(2)KQKC(3)LKAIGSIFGKC(1)[Nle]NKKC(2)KC(3)YPR- |
| | | NH₂ |
| 48 | 49 | H- |
| | | N[Nle]D[Nle]RC(1)SASVEC(2)KQSC(3)LKAIGSIFGKC(1)[Nle]NKKC(2)KC(3)YPR- |
| | | NH₂ |
| 49 | 50 | H-N[Nle]D[Nle]RC(1)SASVEC(2)KQKC(3)KAIGSIFGKC(1)[Nle]NKKC(2)KC(3)YPR- |
| | | NH₂ |
| 50 | 51 | H-NMDMRC(1)SASVEC(2)KQKC(3)YKAIGSIFGKC(1)MNKKC(2)KC(3)YPR-NH₂ |
| 51 | 52 | H-NMDMRC(1)SASVEC(2)KQKC(3)RKAIGSIFGKC(1)MNKKC(2)KC(3)YPR-NH₂ |
| 52 | 53 | H-NMDMRC(1)SASVEC(2)KQKC(3)LAAIGSIFGKC(1)MNKKC(2)KC(3)YPR-NH₂ |
| 53 | 54 | H-NMDMRC(1)SASVEC(2)KQKC(3)LYAIGSIFGKC(1)MNKKC(2)KC(3)YPR-NH₂ |
| 54 | 55 | H-NMDMRC(1)SASVEC(2)KQKC(3)LAIGSIFGKC(1)MNKKC(2)KC(3)YPR-NH₂ |
| 55 | 56 | H-NMDMRC(1)SASVEC(2)KQKC(3)LKYIGSIFGKC(1)MNKKC(2)KC(3)YPR-NH₂ |
| 56 | 57 | H-NMDMRC(1)SASVEC(2)KQKC(3)LKRIGSIFGKC(1)MNKKC(2)KC(3)YPR-NH₂ |
| 57 | 58 | H-NMDMRC(1)SASVEC(2)KQKC(3)LKIGSIFGKC(1)MNKKC(2)KC(3)YPR-NH₂ |
| 58 | 59 | H-NMDMRC(1)SASVEC(2)KQKC(3)LKAYGSIFGKC(1)MNKKC(2)KC(3)YPR-NH₂ |
| 59 | 60 | H-NMDMRC(1)SASVEC(2)KQKC(3)LKAEGSIFGKC(1)MNKKC(2)KC(3)YPR-NH₂ |
| 60 | 61 | H-NMDMRC(1)SASVEC(2)KQKC(3)LKARGSIFGKC(1)MNKKC(2)KC(3)YPR-NH₂ |
| 61 | 62 | H-NMDMRC(1)SASVEC(2)KQKC(3)LKAGSIFGKC(1)MNKKC(2)KC(3)YPR-NH₂ |
| 62 | 63 | H- |
| | | N[Nle]D[Nle]RC(1)SASVEC(2)KQKC(3)LKAIGSPFGKC(1)[Nle]NKKC(2)KC(3)YPR- |
| | | NH₂ |
| 63 | 64 | H- |
| | | N[Nle]D[Nle]RC(1)SASKEC(2)KQKC(3)LKAIGSIFGKC(1)[Nle]NKKC(2)KC(3)YPR- |
| | | NH₂ |
| 64 | 65 | H- |
| | | H[Nle]D[Nle]RC(1)SASVEC(2)KQKC(3)LKAIGSIFGKC(1)[Nle]NKKC(2)KC(3)YPR- |
| | | NH₂ |
| 65 | 66 | H- |
| | | Y[Nle]D[Nle]RC(1)SASVEC(2)KQKC(3)LKAIGSIFGKC(1)[Nle]NKKC(2)KC(3)YPR- |
| | | NH₂ |
| 66 | 67 | H- |
| | | S[Nle]D[Nle]RC(1)SASVEC(2)KQKC(3)LKAIGSIFGKC(1)[Nle]NKKC(2)KC(3)YPR- |
| | | NH₂ |
| 66 | 68 | H- |
| | | S[Nle]D[Nle]RC(1)SASVEC(2)KQKC(3)LKAIGSIFGKC(1)[Nle]NKKC(2)KC(3)YPR- |
| | | OH |
| 67 | 69 | H- |
| | | V[Nle]D[Nle]RC(1)SASVEC(2)KQKC(3)LKAIGSIFGKC(1)[Nle]NKKC(2)KC(3)YPR- |
| | | NH₂ |
| 68 | 70 | H- |
| | | S[Nle]D[Nle]RC(1)SA[Abu]VEC(2)KQKC(3)LKAIGSIFGKC(1)MNKKC(2)KC(3)YPR- |
| | | NH₂ |
| 69 | 71 | H-S[Nle]D[Nle]RC(1)SASVEC(2)KQKC(3)LKAIGSIFG[homo- |
| | | Lys]C(1)MNKKC(2)KC(3)YPR-NH₂ |
| 70 | 72 | H-S[Nle]D[Nle]RC(1)SASVEC(2)KQKC(3)LKAIGSIFGKC(1)MNKKC(2)KC(3)[F(4- |
| | | NH₂)]PR-NH₂ |
| 71 | 73 | H-S[Nle]D[Nle]RC(1)SASVEC(2)GQKC(3)LKAIGSIFGKC(1)MNKKC(2)KC(3)YPR- |
| | | NH₂ |
| 72 | 74 | H-S[Nle]D[Nle]RC(1)SASVEC(2)VQKC(3)LKAIGSIFGKC(1)MNKKC(2)KC(3)YPR- |
| | | NH₂ |
| 73 | 75 | H-S[Nle]D[Nle]RC(1)SASVEC(2)K[2-Amino-5- |
| | | carboxypentanoyl]KC(3)LKAIGSIFGKC(1)MNKKC(2)KC(3)YPR-NH₂ |
| 74 | 76 | H-S[Nle]D[Nle]RC(1)SASVEC(2)KQKC(3)LKAIGSIFGKC(1)MNKKC(2)KC(3)YQ- |
| | | NH₂ |
| 75 | 77 | H-S[Nle]D[Nle]RC(1)SASVEC(2)KQKC(3)LKAIGSIFGKC(1)MNKKC(2)RC(3)YPR- |
| | | NH₂ |
| 76 | 78 | H-S[Nle]DERC(1)SASVEC(2)KQKC(3)LKAIGSIFGKC(1)MNKKC(2)KC(3)YPR-NH₂ |
| 77 | 79 | H-S[Nle]D[Nle]RC(1)SASVEC(2)KQKC(3)LGAIGSIFGKC(1)MNKKC(2)KC(3)YPR- |
| | | NH₂ |
| 78 | 80 | H-S[Nle]D[Nle]RC(1)SASVEC(2)KQKC(3)LVAIGSIFGKC(1)MNKKC(2)KC(3)YPR- |
| | | NH₂ |
| 79 | 81 | H-S[Nle]D[Nle]RC(1)SASVEC(2)AQSC(3)LKAIGSIFGKC(1)MNKKC(2)KC(3)YPR- |
| | | NH₂ |
| 80 | 82 | H-S[Nle]D[Nle]RC(1)SASVEC(2)AQKC(3)LAAIGSIFGKC(1)MNKKC(2)KC(3)YPR- |
| | | NH₂ |
| 81 | 83 | H-S[Nle]D[Nle]RC(1)SASVEC(2)AQLC(3)LAAIGSIFGKC(1)MNKKC(2)KC(3)YPR- |
| | | NH₂ |
| 82 | 84 | H-P[Nle]E[Nle]RC(1)[2,4- |
| | | Diaminobutanoyl]ASVEC(2)KQKC(3)LAAIGSIFGKC(1)[Nle]NKKC(2)KC(3)YPR-OH |
| 83 | 85 | H- |
| | | P[Nle]D[Nle]RC(1)SASVEC(2)KQKC(3)LKAIGC(4)IFGKC(1)[Nle]NKKC(2)KC(3)YP |
| | | C(4)-NH₂ |
| 83 | 86 | H- |
| | | P[Nle]D[Nle]RC(1)SASVEC(2)KQKC(3)LKAIGC(4)IFGKC(1)[Nle]NKKC(2)KC(3)YP |
| | | C(4)-OH |
| 84 | 87 | H- |
| | | S[Nle]D[Nle]RC(1)SASVEC(2)KEKC(3)LQAIGSIFGKC(1)[Nle]NKKC(2)KC(3)YPR- |
| | | NH₂ |
| 85 | 88 | H-P[Nle]D[Nle]RC(1)SASVEC(2)KEKC(3)L[homo- |
| | | Gln]AIGSIFGKC(1)[Nle]NKKC(2)KC(3)YPR-NH₂ |
| 86 | 89 | H-C(1)SASVEC(2)KQKC(3)LKAIGC(4)IFGKC(1)[Nle]NKKC(2)KC(3)YPC(4)-NH₂ |
| 87 | 90 | H- |
| | | S[Nle]D[Nle]RC(1)SASVEC(2)KQKC(3)LAAIGC(4)IFGKC(1)[Nle]NKKC(2)KC(3)YP |
| | | C(4)-NH₂ |
| 88 | 91 | H-P[Nle]E[Nle]RC(1)[2-Amino-3- |
| | | guanidinopropionyl]ASVEC(2)KQKC(3)LAAIGSIFGKC(1)[Nle]NKKC(2)KC(3)YPR- |
| | | OH |
| 89 | 92 | H-S[Nle]D[Nle]RC(1)SALVEC(2)KQKC(3)LKAIGSIFGKC(1)MNKKC(2)KC(3)YPR- |
| | | NH₂ |
| 90 | 93 | H-S[Nle]D[Nle]RC(1)SAVVEC(2)KQKC(3)LKAIGSIFGKC(1)MNKKC(2)KC(3)YPR- |
| | | NH₂ |
| 91 | 94 | H-S[Nle]D[Nle]RC(1)SASVEC(2)KQKC(3)LKAIGSIFGKC(1)MNKKC(2)KC(3)[F(4- |
| | | F)]PR-NH₂ |
| 92 | 95 | H-S[Nle]D[Nle]RC(1)SASVEC(2)KQKC(3)LKAIGSIFGKC(1)MNKKC(2)KC(3)[F(4- |
| | | NO₂)]PR-NH₂ |
| 93 | 96 | H-S[Nle]D[Nle]RC(1)SASVEC(2)KQKC(3)LKAIGSIFGKC(1)MNKKC(2)KC(3)[F(4- |
| | | CH₃)]PR-NH₂ |
| 94 | 97 | H-[Nle]D[Nle]RC(1)SASVEC(2)KQKC(3)LKAIGSIFGKC(1)[Nle]NKKC(2)KC(3)YPR- |
| | | OH |
| 95 | 98 | H-NID[Nle]RC(1)SASVEC(2)KQKC(3)LKAIGSIFGKC(1)[Nle]NKKC(2)KC(3)YPR- |
| | | OH |
| 96 | 99 | H-PIE[Nle]RC(1)SASVEC(2)KQKC(3)LKAIGSIFGKC(1)[Nle]NKKC(2)KC(3)YPR-OH |
| 97 | 100 | H- |
| | | P[Nle]E[Nle]RC(1)SASVEC(2)KQKC(3)LAAIGSIFGKC(1)[Nle]NKKC(2)KC(3)YPR- |
| | | OH |
| 97 | 101 | H- |
| | | P[Nle]E[Nle]RC(1)SASVEC(2)KQKC(3)LAAIGSIFGKC(1)[Nle]NKKC(2)KC(3)YPR- |
| | | NH₂ |
| 98 | 102 | H- |
| | | P[Nle]E[Nle]RC(1)SA[Orn]VEC(2)KQKC(3)LAAIGSIFGKC(1)[Nle]NKKC(2)KC(3)YP |
| | | R-OH |
| 99 | 103 | H- |
| | | P[Nle]E[Nle]RC(1)SASVEC(2)KQKC(3)LLAIGSIFGKC(1)[Nle]NKKC(2)KC(3)YPR- |
| | | OH |
| 100 | 104 | H-PIDERC(1)SASVEC(2)KQKC(3)LAAIGSIFGKC(1)[Nle]NKKC(2)KC(3)YPR-OH |
| 101 | 105 | H-PIE[Nle]RC(1)SASVEC(2)KQKC(3)LAAIGSIFGKC(1)[Nle]NKKC(2)KC(3)YPR-OH |
| 102 | 106 | H-P[Nle]D[Nle]RC(1)SASVEC(2)AQKC(3)LAAIGSIFGKC(1)MNKKC(2)KC(3)YPR- |
| | | OH |
| 103 | 107 | H- |
| | | P[Nle]E[Nle]RC(1)SASVEC(2)AQKC(3)LAAIGSIFGKC(1)[Nle]NKKC(2)KC(3)YPR- |
| | | OH |
| 104 | 108 | H-C(1)SASVEC(2)KQKC(3)LKAIGSIFGKC(1)[Nle]NKKC(2)KC(3)YPR-NH₂ |
| 104 | 109 | Ac-C(1)SASVEC(2)KQKC(3)LKAIGSIFGKC(1)[Nle]NKKC(2)KC(3)YPR-NH₂ |
| 105 | 110 | H-P[Nle]E[Nle]RC(1)[2,3- |
| | | Diaminopropanoyl]ASVEC(2)KQKC(3)LAAIGSIFGKC(1)[Nle]NKKC(2)KC(3)YPR- |
| | | OH |
| 106 | 111 | H-RC(1)SASVEC(2)KQKC(3)LKAIGSIFGKC(1)[Nle]NKKC(2)KC(3)YPR-NH₂ |
| 106 | 112 | Ac-RC(1)SASVEC(2)KQKC(3)LKAIGSIFGKC(1)[Nle]NKKC(2)KC(3)YPR-OH |
| 107 | 113 | Ac-SKC(1)SASVEC(2)KQKC(3)LKAIGSIFGKC(1)[Nle]NKKC(2)KC(3)YPR-NH₂ |
| 108 | 114 | Ac-LRC(1)SASVEC(2)KQKC(3)LKAIGSIFGKC(1)[Nle]NKKC(2)KC(3)YPR-NH₂ |
| 108 | 115 | H-LRC(1)SASVEC(2)KQKC(3)LKAIGSIFGKC(1)[Nle]NKKC(2)KC(3)YPR-OH |
| 109 | 116 | Ac-C(1)SASVEC(2)KQKC(3)LKAIGSIFGKC(1)[Nle]NKKC(2)KC(3)YPS-NH₂ |
| 110 | 117 | Ac-C(1)SASVEC(2)KQKC(3)LKAIGSIFGKC(1)[Nle]NKKC(2)KC(3)YS-NH₂ |
| 111 | 118 | Ac-C(1)SASVEC(2)KQKC(3)LKAIGSIFGKC(1)[Nle]NKKC(2)KC(3)YG-NH₂ |
| 112 | 119 | Ac-C(1)SASVEC(2)KQKC(3)LKAIGSIFGKC(1)[Nle]NKKC(2)KC(3)Y-NH₂ |
| 113 | 120 | H- |
| | | P[Nle]E[Nle]RC(1)[Orn]ASVEC(2)KQKC(3)LAAIGSIFGKC(1)[Nle]NKKC(2)KC(3)YP |
| | | R-OH |
| 114 | 121 | H- |
| | | P[Nle]E[Nle]RC(1)HASVEC(2)KQKC(3)LAAIGSIFGKC(1)[Nle]NKKC(2)KC(3)YPR- |
| | | OH |
| 115 | 122 | Ac-LRC(1)SASVEC(2)KQKC(3)LAAIGSIFGKC(1)[Nle]NKKC(2)KC(3)YPR-OH |
| 116 | 123 | Ac-LRC(1)SASVEC(2)KQKC(3)LAAIGSIFGKC(1)MNKKC(2)KC(3)YPR-OH |
| 117 | 124 | H-C(1)SASVEC(2)KQKC(3)LKAIGSIFGKC(1)MNKKC(2)KC(3)YPR-OH |
| 118 | 125 | H- |
| | | P[Nle]E[Nle]RC(1)SASVEC(2)KEKC(3)LAAIGSIFGKC(1)[Nle]NKKC(2)KC(3)YPR- |
| | | OH |
| 119 | 126 | H- |
| | | p[Nle]E[Nle]RC(1)SASVEC(2)KQKC(3)LAAIGSIFGKC(1)[Nle]NKKC(2)KC(3)YPR- |
| | | OH |
| 120 | 127 | H-P[Nle]E[Nle]RC(1)SASVEC(2)KQKC(3)LAAIGSIFGKC(1)[Nle]NKKC(2)KC(3)YR- |
| | | OH |
| 121 | 128 | H-[Nle]E[Nle]RC(1)SASVEC(2)KQKC(3)LAAIGSIFGKC(1)[Nle]NKKC(2)KC(3)YPR- |
| | | OH |
| 122 | 129 | H- |
| | | P[Nle]E[Nle]RC(1)SASVEC(2)KQKC(3)LAAIGSIFGKC(1)[Nle]NKKC(2)KC(3)YPRR- |
| | | OH |
| 123 | 130 | H- |
| | | P[Nle]E[Nle]RC(1)SASVEC(2)KQKC(3)LAAIGSIFGKC(1)[Nle]NKKC(2)KC(3)YPRY- |
| | | OH |
| 124 | 131 | H- |
| | | P[Nle]E[Nle]RC(1)SASVEC(2)KQKC(3)LAAIGSIFGKC(1)[Nle]NKKC(2)KC(3)YPRL- |
| | | OH |
| 125 | 132 | H- |
| | | P[Nle]E[Nle]RC(1)SASVEC(2)KQKC(3)LAAIGSIFGKC(1)[Nle]NKKC(2)KC(3)YPRH- |
| | | OH |
| 126 | 133 | H- |
| | | P[Nle]E[Nle]RC(1)SASVEC(2)KQKC(3)LAAIGSIFGKC(1)[Nle]NKKC(2)KC(3)YPRE- |
| | | OH |
| 127 | 134 | H- |
| | | P[Nle]E[Nle]RC(1)SASVEC(2)KQKC(3)LAAIGSIFGKC(1)[Nle]NKKC(2)KC(3)YPRK |
| | | S-OH |
| 128 | 135 | H- |
| | | P[Nle]E[Nle]RC(1)SASVEC(2)KQKC(3)LAAIGSIFGKC(1)[Nle]NKKC(2)KC(3)YPRF |
| | | E-OH |
| 129 | 136 | H- |
| | | P[Nle]E[Nle]RC(1)SASVEC(2)KQKC(3)LAAIGSIFGKC(1)[Nle]NKKC(2)KC(3)YPRH |
| | | R-OH |
| 130 | 137 | H- |
| | | P[Nle]E[Nle]RC(1)SASVEC(2)KQKC(3)LAAIGSIFGKC(1)[Nle]NKKC(2)KC(3)YPRA |
| | | K-OH |
| 131 | 138 | H-P[Nle]E[Nle]RC(1)SASVEC(2)KQKC(3)LAAIGSIFGKC(1)[Nle]NKKC(2)KC(3)YP- |
| | | [(4-amino-5-hydroxypentyl)guanidine] |
| 132 | 139 | H- |
| | | P[Nle]E[Nle]RC(1)SASVEC(2)KQKC(3)LAAIGSIFGKC(1)[Nle]NKKC(2)KC(3)YPR- |
| | | [4-amino-5-hydroxypentanamide] |
| 133 | 140 | H- |
| | | P[Nle]E[Nle]RC(1)SASVEC(2)KQKC(3)LAAIGSIFGKC(1)[Nle]NKKC(2)KC(3)YPRS |
| | | T-OH |
| 134 | 141 | H- |
| | | P[Nle]E[Nle]RC(1)SASVEC(2)KQKC(3)LAAIGSIFGKC(1)[Nle]NKKC(2)KC(3)YPRR |
| | | Y-OH |
| 135 | 142 | H- |
| | | P[Nle]E[Nle]RC(1)SSSVEC(2)KQKC(3)LAAIGSIFGKC(1)[Nle]NKKC(2)KC(3)YPR- |
| | | OH |
| 136 | 143 | H- |
| | | P[Nle]E[Nle]RC(1)SLSVEC(2)KQKC(3)LAAIGSIFGKC(1)[Nle]NKKC(2)KC(3)YPR- |
| | | OH |
| 137 | 144 | H- |
| | | P[Nle]E[Nle]RC(1)SAPVEC(2)KQKC(3)LAAIGSIFGKC(1)[Nle]NKKC(2)KC(3)YPR- |
| | | OH |
| 138 | 145 | H- |
| | | P[Nle]E[Nle]RC(1)SASPEC(2)KQKC(3)LAAIGSIFGKC(1)[Nle]NKKC(2)KC(3)YPR- |
| | | OH |
| 139 | 146 | H- |
| | | P[Nle]E[Nle]RC(1)SASQEC(2)KQKC(3)LAAIGSIFGKC(1)[Nle]NKKC(2)KC(3)YPR- |
| | | OH |
| 140 | 147 | H- |
| | | P[Nle]E[Nle]RC(1)SASVEC(2)LQKC(3)LAAIGSIFGKC(1)[Nle]NKKC(2)KC(3)YPR- |
| | | OH |
| 141 | 148 | H- |
| | | P[Nle]E[Nle]RC(1)SASVEC(2)KQPC(3)LAAIGSIFGKC(1)[Nle]NKKC(2)KC(3)YPR- |
| | | OH |
| 142 | 149 | H- |
| | | P[Nle]E[Nle]RC(1)EASVEC(2)KQKC(3)LAAIGSIFGKC(1)[Nle]NKKC(2)KC(3)YPR- |
| | | OH |
| 143 | 150 | H- |
| | | P[Nle]E[Nle]RC(1)FASVEC(2)KQKC(3)LAAIGSIFGKC(1)[Nle]NKKC(2)KC(3)YPR- |
| | | OH |
| 144 | 151 | H- |
| | | P[Nle]E[Nle]RC(1)SYSVEC(2)KQKC(3)LAAIGSIFGKC(1)[Nle]NKKC(2)KC(3)YPR- |
| | | OH |
| 145 | 152 | H- |
| | | P[Nle]E[Nle]RC(1)SAFVEC(2)KQKC(3)LAAIGSIFGKC(1)[Nle]NKKC(2)KC(3)YPR- |
| | | OH |
| 146 | 153 | H-P[Nle]E[Nle]RC(1)SA[2,3- |
| | | Diaminopropanoyl]VEC(2)KQKC(3)LAAIGSIFGKC(1)[Nle]NKKC(2)KC(3)YPR-OH |
| 147 | 154 | H-P[Nle]E[Nle]RC(1)[2,4- |
| | | Diaminobutanoyl]ASVEC(2)AQSC(3)LAAIGSIFGKC(1)[Nle]NGKC(2)KC(3)YPR-OH |
| 147 | 155 | H-P[Nle]E[Nle]RC(1)[2,4- |
| | | Diaminobutanoyl]ASVEC(2)AQSC(3)LAAIGSIFGKC(1)[Nle]NGKC(2)KC(3)YPR- |
| | | NH₂ |
| 148 | 156 | H- |
| | | P[Nle]E[Nle]RC(1)RASVEC(2)AQSC(3)LAAIGSIFGKC(1)[Nle]NGKC(2)KC(3)YPR- |
| | | NH₂ |
| 149 | 157 | H- |
| | | P[Nle]E[Nle]RC(1)SASREC(2)AQSC(3)LAAIGSIFGKC(1)[Nle]NGKC(2)KC(3)YPR- |
| | | NH₂ |
| 150 | 158 | H- |
| | | P[Nle]E[Nle]RC(1)SASVEC(2)KQKC(3)LAKIGSIFGKC(1)[Nle]NKKC(2)KC(3)YPR- |
| | | OH |
